(19) United States
(12) Patent Application Publication
(10) Pub. No.: US 2025/0339271 A1
(43) Pub. Date: Nov. 6, 2025

(54) METHODS AND SYSTEMS FOR REPAIRING A NATIVE VALVE

(71) Applicant: EDWARDS LIFESCIENCES CORPORATION, Irvine, CA (US)

(72) Inventors: Aviv Galon, Tel Aviv (IL); Mark Chau, Laguna Hills, CA (US); Sam Shafigh, Mission Viejo, CA (US); Bar Eytan-Vaisman, Gedera (IL); Amir Geris, Haifa (IL); Nikolai Gurovich, Hadera (IL); David Maimon, Atlit (IL); Michael Bukin, Pardes Hanna (IL); Meir Abucasis, Hadera (IL); Noam Nir, Pardes-Hanna (IL); Ofry Efraim Yossef, Tel-Aviv (IL); Bhumica A. Amin, Modesto, CA (US)

(21) Appl. No.: 19/266,495

(22) Filed: Jul. 11, 2025

Related U.S. Application Data
(63) Continuation of application No. PCT/US2024/010812, filed on Jan. 9, 2024.
(60) Provisional application No. 63/438,532, filed on Jan. 11, 2023.

(51) Int. Cl. A61F 2/24

(52) U.S. Cl. CPC A61F 2/2457; A61F 2/2463; A61F 2/2466; A61F 2220/0016; A61F 2220/0033; A61F 2230/0069

(57) ABSTRACT

An implantable device or implant is configured to be positioned within anatomy such as a native heart valve to repair the valve. The implantable device or implant is configured to be attached to one or more portions of the anatomy or native heart valve, such as one or more portions thereof can be moved closer together. For example, native valve leaflets, chordae tendinea, papillary muscles, a leaflet cleft, etc. can be moved to prevent or inhibit regurgitant flow through the native valve.

### RELATED APPLICATIONS

The present application is a continuation of Patent Cooperation Treaty application number PCT/US2024/010812, filed on Jan. 9, 2024, which claims the benefit of U.S. Provisional Patent Application 63/438,532, filed on Jan. 11, 2023, which are both incorporated herein by reference in their entireties.

### BACKGROUND

The native heart valves (i.e., the aortic, pulmonary, tricuspid, and mitral valves) serve critical functions in assuring the forward flow of an adequate supply of blood through the cardiovascular system. These heart valves may be damaged, and thus rendered less effective, for example, by congenital malformations, inflammatory processes, infectious conditions, disease, etc. Such damage to the valves may result in serious cardiovascular compromise or death. Damaged valves can be surgically repaired or replaced during open heart surgery. However, open heart surgeries are highly invasive, and complications may occur. Transvascular techniques can be used to introduce and implant prosthetic devices in a manner that is much less invasive than open heart surgery. As one example, a transvascular technique useable for accessing the native mitral and aortic valves is the trans-septal technique. The trans-septal technique comprises advancing a catheter into the right atrium (e.g., inserting a catheter into the right femoral vein, up the inferior vena cava and into the right atrium). The septum is then punctured, and the catheter passed into the left atrium. A similar transvascular technique can be used to implant a device within the tricuspid valve that begins similarly to the trans-septal technique but stops short of puncturing the septum and instead turns the delivery catheter toward the tricuspid valve in the right atrium.

A healthy heart has a generally conical shape that tapers to a lower apex. The heart is four-chambered and comprises the left atrium, right atrium, left ventricle, and right ventricle. The left and right sides of the heart are separated by a wall generally referred to as the septum. The native mitral valve of the human heart connects the left atrium to the left ventricle. The mitral valve has a very different anatomy than other native heart valves. The mitral valve includes an annulus portion, which is an annular portion of the native valve tissue surrounding the mitral valve orifice, and a pair of cusps, or leaflets, extending downward from the annulus into the left ventricle. The mitral valve annulus may form a “D”-shaped, oval, or otherwise out-of-round cross-sectional shape having major and minor axes. The anterior leaflet may be larger than the posterior leaflet, forming a generally “C”-shaped boundary between the abutting sides of the leaflets when they are closed together.

When operating properly, the anterior leaflet and the posterior leaflet function together as a one-way valve to allow blood to flow only from the left atrium to the left ventricle. The left atrium receives oxygenated blood from the pulmonary veins. When the muscles of the left atrium contract and the left ventricle dilates (also referred to as “ventricular diastole” or “diastole”), the oxygenated blood that is collected in the left atrium flows into the left ventricle. When the muscles of the left atrium relax and the muscles of the left ventricle contract (also referred to as “ventricular systole” or “systole”), the increased blood pressure in the left ventricle urges the sides of the two leaflets together, thereby closing the one-way mitral valve so that blood cannot flow back to the left atrium and is instead expelled out of the left ventricle through the aortic valve. To prevent or inhibit the two leaflets from prolapsing under pressure and folding back through the mitral annulus toward the left atrium, a plurality of fibrous cords called chordae tendineae tether the leaflets to papillary muscles in the left ventricle.

Valvular regurgitation involves the valve improperly allowing some blood to flow in the wrong direction through the valve. For example, mitral regurgitation occurs when the native mitral valve fails to close properly and blood flows into the left atrium from the left ventricle during the systolic phase of heart contraction. Mitral regurgitation is one of the most common forms of valvular heart disease. Mitral regurgitation may have many different causes, such as leaflet prolapse, dysfunctional papillary muscles, stretching of the mitral valve annulus resulting from dilation of the left ventricle, more than one of these, etc. Mitral regurgitation at a central portion of the leaflets can be referred to as central jet mitral regurgitation and mitral regurgitation nearer to one commissure (i.e., location where the leaflets meet) of the leaflets can be referred to as eccentric jet mitral regurgitation. Central jet regurgitation occurs when the edges of the leaflets do not meet in the middle and thus the valve does not close, and regurgitation is present. Tricuspid regurgitation may be similar, but on the right side of the heart.

### SUMMARY

This summary is meant to provide some examples and is not intended to be limiting of the scope of the invention in any way. For example, any feature included in an example of this summary is not required by the claims, unless the claims explicitly recite the feature. Also, the features, components, steps, concepts, etc. described in examples in this summary and elsewhere in this disclosure can be combined in a variety of ways. Various features and steps as described elsewhere in this disclosure can be included in the examples summarized here.

Systems and devices useable for repairing and/or treating a native valve of a patient are disclosed. The devices can be valve repair devices, valve treatment devices, implantable devices, implants, etc. While sometimes described as an implantable device for illustration purposes in some implementations herein, similar configurations can be used on other devices, e.g., valve repair devices, etc., that are not necessarily implanted and may be removed after treatment.

In some implementations, a device useable for repairing/treating a native valve includes a leaflet coaptation assist element, one or more attachment devices, and a lock. The leaflet coaptation assist element configured to be positioned between a first leaflet and a second leaflet of the native valve to prevent or inhibit the first and second leaflet from prolapsing. In some implementations, the one or more attachment devices are configured to engage and move the first and the second leaflets into a repair position.

In some implementations, the lock is configured to lock the first and the second leaflets in the repair position. In some implementations, the leaflet coaptation assist element is configured to be removed after the lock has been deployed.

In some implementations, the one or more attachment devices include a first anchor configured to engage a ventricular side of the first leaflet, a first line connected to the first anchor and extending through the first leaflet to an atrial side of the first leaflet, a second anchor configured to engage a ventricular side of the second leaflet, and a second line connected to the second anchor and extending through the second leaflet to an atrial side of the second leaflet.

In some implementations, the leaflet coaptation assist element has a delivery state for deploying through a catheter and a deployed state for positioning in the valve to prevent or inhibit prolapsing of the leaflets.

In some implementations, the leaflet coaptation assist element includes a first arm connected to a second arm by a joint portion. In some implementations, the first arm has a first outer surface for engaging the first leaflet and the second arm has a second outer surface of engaging the second leaflet.

In some implementations, the leaflet coaptation assist element is configured for the first line to extend through the first arm and the second line to extend through the second arm.

In some implementations, the device includes a first spacer configured to be positioned between the lock and the first leaflet and a second spacer configured to be positioned between the lock and the second leaflet.

In some implementations, the first spacer is configured to receive the first line therethrough and the second spacer is configured to receive the second line therethrough.

In some implementations, the first retrieval line attached to the first arm and a second retrieval line attached to the second arm to withdraw the leaflet coaptation assist device from between the first and second leaflet after the lock is in place.

In some implementations, the leaflet coaptation assist includes a tubular body having a compressible portion configured to be longitudinally compressed into the deployed state. In some implementations, the tubular body includes a braided material, such as for example, nitinol wire.

In some implementations, the compressible portion forms a spinning top shape when in the deployed state.

In some implementations, the compressible portion includes a distal end attached to an actuation element extending through the compressible portion and a proximal end axially movable relative to the distal end and the actuation element. In some implementations, the proximal end includes a stop configured to engage a positioning element for moving the compressible portion from the delivery state to the deployed state.

In some implementations, the device includes a retaining member configured to engage the first leaflet to retain the first leaflet in position to be engage by the first anchor.

In some implementations, the retaining member includes a stem configured to extend distally from the leaflet coaptation assist element and an arm extending proximally from a distal end of the stem at an acute angle.

In some implementations, the arm is configured to extend to a ventricular side of the first leaflet to position the first leaflet between the arm and the leaflet coaptation assist element.

In some implementations, a method useable for repairing a native heart valve includes preventing or inhibiting a first and a second leaflet from prolapsing with a leaflet coaptation assist element, engaging a first leaflet and a second leaflet with one or more attachment devices and moving the first and second leaflet to a repair position, locking the first and second leaflets in the repair position, and removing the leaflet coaptation assist element.

In some implementations, engaging a first leaflet and a second leaflet with one or more attachment devices includes deploying a first anchor through the first leaflet, deploying a second anchor through the second leaflet, and tensioning a first line connected to the first leaflet and a second line connected to a second leaflet to pull the first and second leaflet towards each other to a repair position.

In some implementations, deploying a first anchor through the first leaflet further comprising deploying the first anchor through an opening in the leaflet coaptation assist element.

In some implementations, the method includes delivering the leaflet coaptation assist element, in a delivery state, through a catheter to the native heart valve, positioning the leaflet coaptation assist element between the first leaflet and the second leaflet, and moving the leaflet coaptation assist to a deployed state for preventing or inhibiting the first and the second leaflet from prolapsing.

In some implementations, the method includes deploying a first spacer to be positioned between the first leaflet and the lock.

In some implementations, the first spacer is configured to receive the first line therethrough.

In some implementations, the method includes engaging a ventricular side of the first leaflet to position the first leaflet for deploying the first anchor through the first leaflet.

In some implementations, removing the leaflet coaptation assist element further comprising pulling one or more retrieval lines attached to the leaflet coaptation assist element.

In some implementations, moving the leaflet coaptation assist to a deployed state further comprises longitudinally compressing a compressible portion of the leaflet coaptation assist.

In some implementations, the compressible portion forms a spinning top shape when in the deployed state.

In some implementations, longitudinally compressing the compressible portion further comprises holding a distal end of the compressible portion in place while pushing a proximal end of the compressible portion toward the distal end.

In some implementations, longitudinally compressing the compressible portion further comprises includes holding a proximal end of the compressible portion in place while pulling a distal end of the compressible portion toward the proximal end.

In some implementations, a device useable for repairing a native valve includes a coaptation element and a first fixation element. In some implementations, the coaptation element is configured to be positioned between at least a first leaflet and a second leaflet of the native valve.

In some implementations, the first fixation element is configured to extend from the coaptation element at a first location and back to the coaptation element at a second location spaced apart from the first location.

In some implementations, the first fixation element is configured to capture at least one of a first leaflet, a chordae tendineae, or both between the first fixation element and the coaptation element.

In some implementations, the first fixation element is a wire configured to move in an arc from the first location to the second location. In some implementations, the first fixation element includes a shape memory alloy.

In some implementations, the first fixation element has a distal piercing tip configured to pierce through the first leaflet. In some implementations, the first fixation element has a distal end the extends into the coaptation element at the second location.

In some implementations, the distal end is configured to pierce an exterior surface of the coaptation element at the second location.

In some implementations, the first fixation element is configured to capture chordae tendineae of the first leaflet without directly capturing the first leaflet.

In some implementations, the coaptation element includes a first pre-formed aperture at the first location and a second pre-formed aperture at the second location.

In some implementations, the device includes a second fixation element configured to extend from the coaptation element at a third location and back to the coaptation element at a fourth location spaced apart from the third location. In some implementations, the second fixation element is configured to capture at least one of a second leaflet, a second chordae tendineae of the second leaflet, or both between the second fixation element and the coaptation element.

In some implementations, the first location and the second location are on a first side of the coaptation element, and the third location and the fourth location are on a second side of the coaptation element, opposite the first side.

In accordance with some implementations, a method useable for repairing/treating a native valve includes positioning a coaptation element between a first leaflet and a second leaflet of the native valve, moving a first fixation element out of the coaptation element at a first location on the coaptation element, capturing a first leaflet, a chordae tendineae of the first leaflet, or a combination thereof between the first fixation element and the coaptation element, and moving the first fixation element back to the coaptation element at a second location on the coaptation element.

In some implementations, capturing the first leaflet, the chordae tendineae of the first leaflet, or a combination thereof further includes moving the first fixation element in an arc around the first leaflet, the chordae tendineae of the first leaflet, or a combination thereof. In some implementations, the chordae tendineae of the first leaflet is captured without capturing the first leaflet.

In some implementations, the method includes piercing the first leaflet with a distal tip of the first fixation element. In some implementations, the method includes piercing the coaptation element with a distal tip of the first fixation element at the first location.

In some implementations, the method includes piercing the coaptation element with a distal tip of the first fixation element at the second location.

In some implementations, the method includes moving a second fixation element out of the coaptation element at a third location on the coaptation element, capturing a second leaflet, a chordae tendineae of the second leaflet, or a combination thereof between the second fixation element and the coaptation element, and moving the second fixation element back to the coaptation element at a fourth location on the coaptation element.

In some implementations, the first location and the second location are on a first side of the coaptation element, and the third location and the fourth location are on a second side of the coaptation element, opposite the first side.

In some implementations, a device useable for repairing or treating anatomy includes a first capture element configured to engage a tissue or a portion of the anatomy (e.g., a first leaflet, a first chorda tendinea, multiple chordae tendineae of a first leaflet, etc.) of the heart valve. In some implementations, the device comprises a second capture element configured to engage a tissue or a portion of the anatomy (e.g., a second leaflet, a second chorda tendinea, multiple chordae tendineae of a second leaflet, etc.) of the heart valve.

In some implementations, the first capture element and the second capture element are configured to be drawn toward each other to pull the tissue (e.g., to pull a first leaflet and a second leaflet, to pull chordae tendineae of a first leaflet and the second leaflet, etc.) toward each other.

In some implementations, the first capture element comprises and/or is configured as a first cylindrical rod and the second capture element comprises and/or is configured as a second rod.

In some implementations, the first capture element has a first end and a second end opposite the first end and the second capture element has a third end and a fourth end opposite the third end.

In some implementations, the device includes a first attachment element connecting the first end to the third end and configured to draw the first end toward the third end. In some implementations, the first attachment element includes a suture line.

In some implementations, the device includes a second attachment element connecting the second end to the fourth end. In some implementations, the second attachment element is configured to draw the second end toward the fourth end.

In some implementations, the second end is connected to the fourth end by a joint portion. In some implementations, the joint portion includes a hinge. In some implementations, the first capture element and the second capture element are configured to pivot between an open position and a repair position.

In some implementations, the first end and the third end are configured to attach. In some implementations, the first end includes a latch configured to attach to a projection on the third end.

In some implementations, a method useable for repairing/treating anatomy includes positioning a first capture element to engage a first tissue or first portion (e.g., a first leaflet, a first chorda tendinea, first chordae tendineae of a first leaflet, etc.) of the anatomy. In some implementations, the method includes positioning a second capture element to engage a second tissue or second portion (e.g., a second leaflet, a second chorda tendinea, second chordae tendineae of a second leaflet, etc.) of the anatomy.

In some implementations, moving the first capture element toward the second capture element to pull the first tissue/portion (e.g., first chordae tendineae, etc.) toward the second tissue/portion (e.g., second chordae tendineae, etc.) to a repair position.

In some implementations, moving the first capture element toward the second capture element includes pivoting the first capture element relative to the second capture element.

In some implementations, the method includes latching the first capture element to the second capture element in the repair position. In some implementations, the method includes securing the first capture element to the second capture element in the repair position with one or more attachment elements.

In some implementations, the method includes connecting the first capture element to the second capture element by one or more attachment elements. In some implementations, the one or more attachment elements include a suture line. In some implementations, moving the first capture element toward the second capture element includes cinching the suture line.

In some implementations, connecting the first capture element to the second capture element by one or more attachment elements includes connecting a first end of the first capture element to a third end of the second capture element with one of the one or more attachment elements and connecting a second end of the first capture element to a fourth end of the second capture element with another of the one or more attachment elements.

In some implementations, a device useable for repairing/treating a native heart valve incudes a frame including a plurality of interconnected strut and means for attaching to leaflet tissue positioned on the struts.

In some implementations, the means for attaching to leaflet tissue comprises a plurality of spikes. In some implementations, the plurality of struts define a plane and the plurality of spikes extend coplanar to the plane.

In some implementations, plurality of spikes extend from both a first side and a second side of at least one of the plurality of struts. In some implementations, the plurality of spikes extend from only one side of at least one of the plurality of struts.

In some implementations, the plurality of struts form one or more diamond shapes. In some implementations, plurality of struts form one or more rectangular shapes. In some implementations, the plurality of struts form one or more circular shapes.

In some implementations, frame is configured to be folded along a mid-line prior to being positioned between a first leaflet and a second leaflet of the native heart valve.

In some implementations, a method of repairing/treating a native heart valve includes positioning a repair device between a first leaflet and a second leaflet of the heart valve such that the first leaflet and the second leaflet contact the repair device during systole and passively attaching the first leaflet and the second leaflet to the repair device upon the first leaflet and the second leaflet contacting the repair device.

In some implementations, passively attaching the first leaflet to the repair device includes a portion of the device penetrating into the first leaflet. In some implementations, the portion of the device comprises one or more spikes.

In some implementations, a device useable for repairing a native valve includes one or more of a first paddle, a first capture element, and/or a second capture element. In some implementations, the first paddle is moveable between an open position and a closed position.

In some implementations, the device includes an optional second paddle that is movable between an open position and a closed position.

In some implementations, the first capture element is attached to the first paddle and movable between an elongated configuration and an expanded configuration. In some implementations, the first capture element is configured to engage tissue (e.g., a first leaflet, a first chorda tendinea, multiple first chordae tendineae of a first leaflet, etc.) of the heart valve when in the elongated configuration.

In some implementations, the second capture element is attached to the second paddle and movable between an elongated configuration and an expanded configuration. In some implementations, the second capture element is configured to engage tissue (e.g., a second leaflet, a second chorda tendinea, multiple second chordae tendineae of a second leaflet, etc.) of the heart valve when in the elongated configuration.

In some implementations, movement of the pair of paddles to the closed position causes the first capture element and the second capture element to be drawn toward each other to pull the first tissue (e.g., first chordae tendineae of the first leaflet, etc.) and the second tissue (e.g., second chordae tendineae of the second leaflet, etc.) toward each other.

In some implementations, the first capture element has a first connection element, a second connection element, a first flexible portion and a second flexible portion, where the first and second flexible portions are each connected to both of the first and second connection elements.

In some implementations, at least one of the first connection element and the second connection element are movable within a slot of the first paddle to move the first capture element between the elongated and expanded configurations.

In some implementations, the first connection element is a first distance away from the second connection element when the first capture element is in the elongated configuration and the first connection element is a second distance away from the second connection element when the first capture element is in the expanded configuration, where the first distance is greater than the second distance.

In some implementations, the first connection element, the second connection element, the first flexible portion, and the second flexible portion are integrally formed.

In some implementations, the first and second capture elements are normally in the expanded configuration.

In some implementations, the first and second paddles are integrally formed.

In some implementations, the first paddle includes a first hinge portion, and the second paddle includes a second hinge portion, where the first and second hinge portions allow for the first and second paddles to be moved between the opened and closed positions.

In some implementations, the first paddle, the second paddle, the first capture element, and the second capture element are integrally formed.

In some implementations, a method useable for repairing/treating anatomy includes positioning a repair/treatment device proximate the anatomy and positioning a first capture element to engage a first tissue or first portion of the anatomy (e.g., a first leaflet, first chordae tendineae, first chordae tendineae of a first leaflet, etc.) of the native heart valve with the first capture element in an elongated configuration. In some implementations, the method can also include positioning a second capture element to engage a second tissue or second portion of the anatomy (e.g., a second leaflet, second chordae tendineae, second chordae tendineae of a second leaflet, etc.) of the native heart valve with the second capture element in an elongated configuration.

In some implementations, the method includes moving the first capture element to the expanded configuration.

In some implementations, the method includes moving the second capture element to the expanded configuration.

In some implementations, the method includes moving the first and second capture elements toward each other and pull the first tissue or first portion (e.g., first chordae tendineae, etc.) and the second tissue or second portion (e.g., second chordae tendineae, etc.) toward each other.

In some implementations, the method includes moving a first paddle and/or optionally, a second paddle to a closed position such that the first and second capture elements move toward each other and pull the first tissue/portion (e.g., first chordae tendineae, etc.) and the second tissue/portion (e.g., second chordae tendineae, etc.) toward each other.

In some implementations, the first capture element has a first connection element, a second connection element, a first flexible portion and a second flexible portion, where the first and second flexible portions are each connected to both of the first and second connection elements.

In some implementations, the method includes moving at least one of the first connection element and the second connection element within a slot of the first paddle to move the first capture element from the elongated configuration to the expanded configuration.

In some implementations, the first connection element is a first distance away from the second connection element when the first capture element is in the elongated configuration and the first connection element is a second distance away from the second connection element when the first capture element is in the expanded configuration, where the first distance is greater than the second distance.

In some implementations, the first connection element, the second connection element, the first flexible portion, and the second flexible portion are integrally formed.

In some implementations, the first and second capture elements are normally in the expanded configuration.

In some implementations, the method includes engaging the first capture element with one or more actuation elements to move the first capture element to the elongated configuration prior to positioning the first capture element to engage the first tissue (e.g., first chordae tendineae, etc.).

In some implementations, the moving the first paddle and the second paddle to the closed position includes engaging one or more hinge portions of the first and second paddle with an actuation element.

In some implementations, the first and second paddles are integrally formed.

In some implementations, the first and second paddles include Nitinol.

In some implementations, the first paddle, the second paddle, the first capture element, and the second capture element are integrally formed.

In some implementations, the method includes pulling a free end of the first capture element to move the first capture element to the elongated configuration and pulling a free end of the second capture element to move the second capture element to the elongated configuration.

In some implementations, a device useable for repairing/treating anatomy (e.g., a native valve, etc.) includes at least one or both a first capture element and a second capture element.

In some implementations, the first capture element includes a first engagement portion and a second engagement portion. In some implementations, the first engagement portion is configured to engage a first portion of a first tissue (e.g., a first leaflet, first chordae tendineae, first chordae tendineae of a first leaflet, etc.). In some implementations, the second engagement portion is configured to engage a first portion of a second tissue (e.g., a second leaflet, second chordae tendineae, second chordae tendineae of a second leaflet, etc.).

In some implementations, the second capture element includes a first engagement portion and a second engagement portion. In some implementations, the first engagement portion is configured to engage a second portion of the first tissue (e.g., a second portion of a first leaflet, a second portion of first chordae tendineae of a first leaflet, etc.). In some implementations, the second engagement portion is configured to engage a second portion of the second tissue (e.g., a second portion of a second leaflet, a second portion of second chordae tendineae of a second leaflet, etc.).

In some implementations, the first and second capture elements are rotatably coupled such that the first and second capture elements are movable between a delivery configuration and a capture configuration.

In some implementations, the first engagement portion of the first capture element and the second engagement portion of the second capture element secure the first portion of the first tissue (e.g., first portion of the first chordae tendineae, etc.) and the second portion of the second tissue (e.g., second portion of the second chordae tendineae, etc.) in a first capture area when in the capture configuration.

In some implementations, the second engagement portion of the first capture element and the first engagement portion of the second capture element secure the first portion of the second tissue (e.g., first portion of second chordae tendineae, etc.) and the second portion of the first tissue (e.g., first portion of first chordae tendineae, etc.) in a second capture area when in the capture configuration.

In some implementations, the first and second capture elements are rotatably connected by a hinge connection.

In some implementations, the device comprises a hub portion connected to each of the first and second capture elements.

In some implementations, the hub portion is configured to receive an actuation element for moving the first and second capture elements between the delivery and capture configurations.

In some implementations, the first capture element is configured to rotate in a counterclockwise direction to move from the delivery configuration to the capture configuration, and the second capture element is configured to rotate in a clockwise direction to move from the delivery configuration to the capture configuration.

In some implementations, the first and second engagement portions of the first capture element are integrally formed.

In some implementations, the first and second capture elements are configured to rotate between about 75 degrees and about 105 degrees to move from the delivery configuration to the capture configuration.

In some implementations, the first and second capture elements are normally in the capture configuration.

In some implementations, the first engagement portion of the first capture element is longer than the second engagement portion of the first capture element, and the second engagement portion of the second capture element is longer than the first engagement portion of the second capture element.

In some implementations, a method useable for repairing/treating anatomy (e.g., a native valve, etc.) includes positioning a repair/treatment device in a delivery configuration proximate a space or gap between two portions or tissues of the anatomy (e.g., a gap or space between adjacent leaflets of a native valve, etc.). In some implementations, first and second engagement portions of each of first and second capture elements are positioned substantially perpendicular to the gap or space when in the delivery configuration.

In some implementations, the method further includes rotating at least one of the first and second capture elements to a capture configuration. In some implementations, the first capture element is rotated in a first direction such that the first engagement portion engages a first portion of a first tissue (e.g., a first portion of a first leaflet, a first portion of first chordae tendineae of a first leaflet, etc.) and the second engagement portion engages a first portion of a second tissue (e.g., a first portion of a second leaflet, a first portion of second chordae tendineae of a second leaflet, etc.).

In some implementations, the second capture element is rotated in a second direction that is opposite the first direction such that the first engagement portion engages a second portion of the first tissue (e.g., a second portion of the first leaflet, a second portion of first chordae tendineae of the first leaflet, etc.) and the second engagement portion engages a second portion of the second tissue (e.g., a second portion of the second leaflet, a second portion of second chordae tendineae of the second leaflet, etc.).

In some implementations, the first engagement portion of the first capture element and the second engagement portion of the second capture element secures the first portion of the first tissue (e.g., the first portion of first chordae tendineae, etc.) and the second portion of the second tissue (e.g., the second portion of second chordae tendineae, etc.) in a first capture area when in the capture configuration.

In some implementations, the second engagement portion of the first capture element and the first engagement portion of the second capture element secures the first portion of the second tissue (e.g., the first portion of the first chordae tendineae, etc.) and the second portion of the first tissue (e.g., the second portion of the second chordae tendineae, etc.) in a first capture area when in the capture configuration.

In some implementations, the first and second capture elements are rotatably connected by a hinge connection.

In some implementations, the valve repair device includes a hub portion connected to each of the first and second capture elements.

In some implementations, the hub portion is configured to receive an actuation element for moving the first and second capture elements between the delivery and capture configurations.

In some implementations, the first direction is a counterclockwise direction, and the second direction is a clockwise direction.

In some implementations, the first and second engagement portions of the first capture element are integrally formed.

In some implementations, the first and second capture elements include Nitinol.

In some implementations, the first and second capture elements are configured to rotate between about 75 degrees and about 105 degrees to move from the delivery configuration to the capture configuration.

In some implementations, the first and second capture elements are normally in the capture configuration.

In some implementations, the first engagement portion of the first capture element is longer than the second engagement portion of the first capture element, and the second engagement portion of the second capture element is longer than the first engagement portion of the second capture element.

In some implementations, a device usable for repairing/treating anatomy (e.g., a native valve, etc.) includes a base portion and one or more capture elements. In some implementations, each of the one or more capture elements are pivotably connected to the base portion such that each of the one or more capture elements are movable between a delivery configuration and a capture configuration.

In some implementations, a first capture element is configured to engage a first portion of a first tissue (e.g., a first leaflet, first chordae tendineae of a first leaflet, etc.) when moved from the delivery configuration to the capture configuration.

In some implementations, a second capture element is configured to engage a first portion of a second tissue (e.g., second leaflet, second chordae tendineae of a second leaflet, etc.) when moved from the delivery configuration to the capture configuration.

In some implementations, a third capture element is configured to engage a second portion of the first tissue (e.g., a second portion of first chordae tendineae of the first leaflet, etc.) when moved from the delivery configuration to the capture configuration.

In some implementations, a fourth capture element is configured to engage a second portion of the second tissue (e.g., a second portion of second chordae tendineae of the second leaflet, etc.) when moved from the delivery configuration to the capture configuration.

In some implementations, the first and second capture elements secure the first portion of the first tissue (e.g., a first portion of first chordae tendineae, etc.) and the first portion of the second tissue (e.g., a first portion of second chordae tendineae, etc.) in a first capture area when in the capture configuration.

In some implementations, the third and fourth capture elements secure the second portion of the first tissue (e.g., a second portion of second chordae tendineae, etc.) and the second portion of the second tissue (e.g., a second portion of second chordae tendineae, etc.) in a second capture area when in the capture configuration.

In some implementations, the first and third capture elements are integrally formed, and the second and fourth capture elements are integrally formed.

In some implementations, the base portion is configured to receive one or more actuation elements for moving the one or more capture elements between the delivery and capture configurations.

In some of the implementations, each of the capture elements are normally in the capture configuration.

In some implementations, each of the capture elements can be moved from the delivery configuration to the capture configuration independently.

In some implementations, a method useable for repairing/treating anatomy (e.g., a native valve, etc.) includes positioning a device (e.g., a valve repair device, a valve treatment device, a repair device, a treatment device, etc.) in a delivery configuration proximate a gap or space between two portions/tissues of the anatomy (e.g., proximate a gap or space between adjacent leaflets of a native valve, etc.). In some implementations, one or more capture elements are pivotally attached to a base portion of the device are positioned substantially perpendicular to the gap or space.

In some implementations, the method further includes pivoting a pair of capture elements toward each other to a capture configuration such that the pair of capture elements define a capture area.

In some implementations, a first capture element of the pair of capture elements engages a portion of a first tissue (e.g., a portion of a first leaflet, a portion of first chordae tendineae of a first leaflet, a portion of a first leaflet, etc.). In some implementations, a second capture element of the pair of capture elements engages a portion of a second tissue (e.g., a portion of a second leaflet, a portion of second chordae tendineae of a second leaflet, a portion of a second leaflet, etc.).

In some implementations, the portion of the first tissue and/or the portion of the second tissue are secured within the capture area.

In some implementations, the method includes pivoting a second pair of capture elements toward each other to a capture configuration such that the second pair of capture elements define a second capture area. In some implementations, a third capture element of the second pair of capture elements engages a second portion of the first tissue (e.g., a second portion of chordae tendineae of a first leaflet, a second portion of a first leaflet, etc.). In some implementations, a fourth capture element of the second pair of capture elements engages a second portion of the second tissue (e.g., a second portion of chordae tendineae of the second leaflet, a second portion of the second leaflet, etc.).

In some implementations, the second portion of the first tissue and/or the second portion of the second tissue are secured within the second capture area.

In some implementations, the first and third capture elements are integrally formed, and the second and fourth capture elements are integrally formed.

In some implementations, the base portion is configured to receive one or more actuation elements for moving the plurality of capture elements between the delivery and capture configurations.

In some implementations, each of the capture elements comprise Nitinol.

In some of the implementations, each of the capture elements are normally in the capture configuration.

In some implementations, each of the capture elements can be moved from the delivery configuration to the capture configuration independently.

In some implementations, a device useable for repairing a native valve includes one or more paddles, a first capture element, and a second capture element. In some implementations, the one or more paddles includes a first paddle and a second paddle that are movable between open and closed positions.

In some implementations, the first capture element is attached to the first paddle and includes a first housing and a first engagement element at least partially disposed within the first housing. In some implementations, the first engagement element is moveable between a compressed configuration and an expanded configuration.

In some implementations, the second capture element is attached to the second paddle and includes a second housing and a second engagement element at least partially disposed within the second housing. In some implementations, the second engagement element is moveable between a compressed configuration and an expanded configuration.

In some implementations, movement of the pair of paddles to the closed position causes the first engagement element of the first capture element to engage a first tissue (e.g., a first leaflet, first chordae tendineae of a first leaflet, etc.) when in the expanded configuration. In some implementations, movement of the pair of paddles to the closed position causes additionally or alternatively causes the second engagement element of the second capture element to engage a second tissue (e.g., a second leaflet, second chordae tendineae of a second leaflet, etc.) when in the expanded configuration.

In some implementations, the first portion of the first capture element includes a first engagement portion and a second engagement portion, where the first and second engagement portions are compressed towards each other when the first engagement element is in the compressed configuration. In some implementations, the first and second engagement portions extend away from each other when the first engagement element is in the expanded configuration.

In some implementations, the first housing includes a channel for receiving and engaging the first engagement element.

In some implementations, the channel includes one or more sloped walls for facilitating movement of the first engagement element between the compressed and expanded configurations.

In some implementations, the first housing has a first opening for allowing the first engagement portion of the first engagement element to move to the expanded configuration and a second opening for allowing the second engagement portion of the first engagement element to move to the expanded configuration.

In some implementations, each of the first paddle and the second paddle includes an inner paddle portion and an outer paddle portion.

In some implementations, the device includes a first paddle frame connected to the first paddle and the cap, and includes a second paddle frame connected to the second paddle and the cap.

In some implementations, a method useable for repairing/treating a native heart valve or other anatomy includes positioning a repair device proximate the native heart valve or other anatomy. In some implementations, the method includes positioning a first capture element to engage a first tissue (e.g., a first leaflet, first chordae tendineae of a first leaflet, etc.) when an engagement element of the first capture element is in a compressed position relative to a housing of the first capture element.

In some implementations, the method includes positioning a second capture element to engage a second tissue (e.g., a second leaflet, second chordae tendineae of a second leaflet, etc.) when an engagement element of the second capture element is in a compressed position relative to a housing of the second capture element.

In some implementations, the first tissue comprises first chordae tendineae, and the method further includes moving the first capture element to the expanded configuration such that the first engagement element of the first capture element extends behind multiple chordae of the first chordae tendineae.

In some implementations, the second tissue comprises second chordae tendineae, and the method includes moving the second capture element to the expanded configuration such that the second engagement element of the second capture element extends behind multiple chordae of the second chordae tendineae.

In some implementations, the method includes moving the first and second paddles to a closed position such that the first and second capture elements move toward each other and pull the first and second tissues toward each other.

In some implementations, the first portion of the first capture element includes a first engagement portion and a second engagement portion, where the first and second engagement portions are compressed towards each other when the first engagement element is in the compressed configuration, and where the first and second engagement portions extend away from each other when the first engagement element is in the expanded configuration.

In some implementations, the first housing includes a channel for receiving and engaging the first engagement element.

In some implementations, the channel includes one or more sloped walls for facilitating movement of the first engagement element between the compressed and expanded configurations.

In some implementations, the first housing has a first opening for allowing the first engagement portion of the first engagement element to move to the expanded configuration and a second opening for allowing the second engagement portion of the first engagement element to move to the expanded configuration.

In some implementations, each of the first paddle and the second paddle includes an inner paddle portion and an outer paddle portion.

In some implementations, the device includes a first paddle frame connected to the first paddle and the cap, and includes a second paddle frame connected to the second paddle and the cap.

In some implementations, a device useable for repairing/treating anatomy (e.g., a native valve, etc.) includes a first clip member for attaching to a first portion of the anatomy and a second clip member for attaching to a second portion of the anatomy. In some implementations, the first clip member includes a first clip body and a first attachment portion. In some implementations, the second clip member includes a second clip body and a second attachment portion.

In some implementations, the first and second clip members are movable between a disengaged position and an engaged position relative to each other.

In some implementations, the first and second clip members are configured such that, after the first clip member is attached to the first portion of the anatomy and the second clip member is attached to the second portion of the anatomy movement of the first and second clip members to the engaged position causes a region between the first and second portions of the leaflet to be at least one of closed, reduced, eliminated, plicated, etc.

In some implementations, the device further comprises one or more actuation elements for moving the first and second clip members from the disengaged position to the engaged position.

In some implementations, the one or more actuation elements comprises one or more springs that cause the first and second clip members to be in a normally engaged position.

In some implementations, the one or more actuation elements comprise a shape set material that causes the first and second clip members to be in a normally closed position.

In some implementations, the one or more actuation elements comprises NiTi wire that is configured to move the first and second clip members to the engaged position when heated.

In some implementations, the one or more actuation elements comprise a bifurcated member and a tube that engages the bifurcated member to move the first and second clip members to the engaged position.

In some implementations, the one or more actuation elements comprise a suture that is threaded through each of the first and second clip members.

In some implementations, at least one of the first and second clip members has one or more locking features that secure the first clip member to the second clip member when the first and second clip members are in the engaged position.

In some implementations, the first clip member has a first locking protrusion and a first locking receptacle, wherein the second clip member has a second locking protrusion and a second locking receptacle. In some implementations, the first locking receptacle is configured to receive the second locking protrusion. In some implementations the second locking receptacle is configured to receive the first locking protrusion.

In some implementations, a device useable for repairing/treating a native valve includes a first clip member for attaching to a first portion of a leaflet of the native valve and a second clip member for attaching to a second portion of the leaflet of the native valve. In some implementations, the first clip member includes a first clip body and a first attachment portion. In some implementations, the second clip member includes a second clip body and a second attachment portion.

In some implementations, the first and second clip members are movable between a disengaged position and an engaged position relative to each other.

In some implementations, the first and second clip members are configured such that, after the first clip member is attached to the first portion of the leaflet and the second clip member is attached to the second portion of the leaflet, movement of the first and second clip members to the engaged position causes a cleft or gap between the first and second portions of the leaflet to be closed or reduced, e.g., to prevent or reduce regurgitation therethrough.

In some implementations, the device further comprises one or more actuation elements for moving the first and second clip members from the disengaged position to the engaged position.

In some implementations, the one or more actuation elements comprises one or more springs that cause the first and second clip members to be in a normally engaged position.

In some implementations, the one or more actuation elements comprise a shape set material that causes the first and second clip members to be in a normally closed position.

In some implementations, the one or more actuation elements comprises NiTi wire that is configured to move the first and second clip members to the engaged position when heated.

In some implementations, the one or more actuation elements comprise a bifurcated member and a tube that engages the bifurcated member to move the first and second clip members to the engaged position.

In some implementations, the one or more actuation elements comprise a suture that is threaded through each of the first and second clip members.

In some implementations, at least one of the first and second clip members has one or more locking features that secure the first clip member to the second clip member when the first and second clip members are in the engaged position.

In some implementations, the first clip member has a first locking protrusion and a first locking receptacle, wherein the second clip member has a second locking protrusion and a second locking receptacle. In some implementations, the first locking receptacle is configured to receive the second locking protrusion. In some implementations the second locking receptacle is configured to receive the first locking protrusion.

Any of the above method(s) and any methods of using the systems, assemblies, apparatuses, devices, etc. herein can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can optionally comprise computerized and/or physical representations.

Any of the above systems, assemblies, devices, apparatuses, components, etc. can be sterilized (e.g., with heat, radiation, ethylene oxide, hydrogen peroxide, etc.) to ensure they are safe for use with patients, and the methods herein can comprise (or additional methods comprise or consist of) sterilization of one or more systems, devices, apparatuses, components, etc. herein (e.g., with heat, radiation, ethylene oxide, hydrogen peroxide, etc.).

A further understanding of the nature and advantages of the present invention are set forth in the following description and claims, particularly when considered in conjunction with the accompanying drawings in which like parts bear like reference numerals.

### BRIEF DESCRIPTION OF THE DRAWINGS

To further clarify various aspects of examples in the present disclosure, a more particular description of certain examples and implementations will be made by reference to various aspects of the appended drawings. These drawings depict only example implementations of the present disclosure and are therefore not to be considered limiting of the scope of the disclosure. Moreover, while the figures can be drawn to scale for some examples, the figures are not necessarily drawn to scale for all examples. Examples and other features and advantages of the present disclosure will be described and explained with additional specificity and detail through the use of the accompanying drawings in which:

FIG. 1 illustrates a cutaway view of the human heart in a diastolic phase;

FIG. 2 illustrates a cutaway view of the human heart in a systolic phase;

FIG. 3 illustrates a cutaway view of the human heart in a systolic phase showing valve regurgitation;

FIG. 4 is the cutaway view of FIG. 3 annotated to illustrate a natural shape of mitral valve leaflets in the systolic phase;

FIG. 5 illustrates a healthy mitral valve with the leaflets closed as viewed from an atrial side of the mitral valve;

FIG. 6 illustrates a dysfunctional mitral valve with a visible gap between the leaflets as viewed from an atrial side of the mitral valve;

FIG. 7 illustrates a tricuspid valve viewed from an atrial side of the tricuspid valve;

FIGS. 8-14 show an example of an implantable device or implant, in various stages of deployment;

FIG. 15 shows an example of an implantable device or implant that is similar to the device illustrated by FIGS. 8-14, but where the paddles are independently controllable;

FIGS. 16-21 show the example device or implant of FIGS. 8-14 being delivered and deployed within a native valve;

FIG. 22 shows a perspective view of an example device or implant in a closed position;

FIG. 23 shows a perspective view of an example device or implant in a closed position;

FIG. 24 illustrates an example valve repair device with paddles in an open position;

FIG. 25A illustrates another example valve repair device with paddles in a closed position;

FIG. 25B illustrates a top view of an example valve repair device;

FIG. 26 illustrates a perspective view of an example device having paddles of adjustable widths;

FIG. 27 is a cross-section of the implantable device of FIG. 26 in which the implantable device is bisected;

FIG. 28 is another cross-section of the implantable device of FIG. 26 in which the implantable device is bisected along a plane perpendicular to the plane illustrated in FIG. 28;

FIG. 29 is a schematic illustration of an example implant catheter assembly coupled to an implantable device in which an actuation element is coupled to a paddle actuation control and to a driver head of the implantable device;

FIG. 30 is an illustration of the assembly of FIG. 29 with the implantable device rotated 90 degrees to show the paddle width adjustment element coupled to an inner end of the connector of the implantable device and coupled to a paddle width control;

FIGS. 31-34 illustrate an example method and device useable for repairing a heart valve;

FIG. 35 illustrates an example leaflet coaptation assist element for a device useable for repairing a heart valve;

FIG. 36 illustrates the example leaflet coaptation assist element of FIG. 35 in a delivery state;

FIG. 37 illustrates the example leaflet coaptation assist element of FIG. 35 in a deployed state;

FIGS. 38-44 illustrates an example method and device useable for repairing a heart valve utilizing the leaflet coaptation assist element of FIG. 35;

FIG. 45 illustrates an example device installed in a heart valve to engage the leaflets of the heart valve;

FIGS. 46-49 illustrate a top view of the example device of FIG. 45 in various stages of deployment;

FIG. 50 illustrates an example device installed in a heart valve to engage the leaflets of the valve;

FIG. 51 illustrates an example device useable for repairing a heart valve;

FIG. 52 illustrates the example device of FIG. 51 installed in a heart valve;

FIG. 53 illustrates an example device useable for repairing a heart valve;

FIG. 54 illustrates the example device of FIG. 53 installed in a heart valve;

FIG. 55 illustrates an example device useable for repairing a heart valve;

FIGS. 56-58 illustrate an example of a method and device useable for repairing a heart valve where the device is positioned at the commissure of the anterior leaflet and the septal leaflet of a tricuspid valve;

FIG. 59 illustrates the device of FIG. 56-58 positioned at a mid-portion of a tricuspid valve between the anterior leaflet and the septal leaflet;

FIG. 60 illustrates the device of FIG. 56-58 positioned at the mid-portion of a mitral valve;

FIGS. 61A-61I illustrate various example configurations of the device of FIGS. 56-60;

FIGS. 62-64 illustrate the device of FIG. 61A in a heart valve;

FIG. 65 illustrates an example device useable for repairing a heart valve;

FIG. 66 illustrates an example capture element of the example device of FIG. 65, where the capture element is shown in an expanded configuration.

FIG. 67 illustrates the example capture element of FIG. 66, where the capture element is shown in an elongated configuration;

FIGS. 68-71 illustrate an example method for installing the example device of FIG. 65 in a heart valve;

FIG. 72 illustrates the example device of FIG. 65 installed in a heart valve;

FIGS. 73-75 illustrate the example device of FIG. 65 installed in different positions relative to the heart valve.

FIG. 76 illustrates an example device useable for repairing a heart valve;

FIG. 77 illustrates the example device of FIG. 76 with example capture elements of the device shown in an expanded configuration;

FIG. 78 illustrates an example device useable for repairing a heart valve;

FIGS. 79-83 illustrate an example method for installing the example device of FIG. 78 in a heart valve;

FIG. 84 illustrates the example device of FIG. 78 installed in a heart valve;

FIG. 85 illustrates an example device useable for repairing a heart valve;

FIGS. 86-88 illustrate an example method for installing the example device of FIG. 85 in a heart valve;

FIG. 89 illustrates the example device of FIG. 85 installed in a heart valve;

FIG. 90 illustrates an example device useable for repairing a heart valve;

FIGS. 91-92 illustrate an example method for installing the example device of FIG. 90 in a heart valve;

FIG. 93 illustrates the example device of FIG. 90 installed in a heart valve;

FIG. 94 illustrates an example device useable for repairing a heart valve;

FIGS. 95-96 illustrate an example method for installing the example device of FIG. 94 in a heart valve;

FIG. 97 illustrates the example device of FIG. 94 installed in a heart valve;

FIG. 98 illustrates an example device useable for repairing a heart valve;

FIGS. 99-102 illustrate an example capture element for the example device of FIG. 98;

FIG. 103 illustrates example paddles for the example device of FIG. 98;

FIG. 104 illustrates an example paddle frame for the example device of FIG. 98;

FIG. 105 illustrates the example device of FIG. 98, where the device includes example connection elements for connecting the example paddles of FIG. 103 to the example paddle frame of FIG. 104;

FIGS. 106-108 illustrate various portions of an example cap for the example device of FIG. 98;

FIGS. 109-110 illustrate the example device of FIG. 98 installed in a heart valve;

FIG. 111 illustrates a mitral valve viewed from an atrial side of the mitral valve;

FIG. 112 illustrates a posterior leaflet of a mitral valve from a ventricular side of the mitral valve;

FIGS. 113-114 illustrate an example device useable for repairing a heart valve and an example method for installing the example device in the heart valve;

FIGS. 115-116 illustrate an example device useable for repairing a heart valve and an example method for installing the example device in the heart valve;

FIGS. 117-118 illustrate an example device useable for repairing a heart valve; and

FIG. 119 illustrates an example device useable for repairing a heart valve and an example method for installing the example device in the heart valve.

### DETAILED DESCRIPTION

The following description refers to the accompanying drawings, which illustrate example implementations of the present disclosure. Other implementations having different structures and operation do not depart from the scope of the present disclosure.

Example implementations of the present disclosure are directed to systems, devices, methods, etc. useable for repairing a defective heart valve. For example, various implementations of valve repair devices, implantable devices, implants, and systems (including systems for delivery thereof) are disclosed herein, and any combination of these options can be made unless specifically excluded. In other words, individual components of the disclosed devices and systems can be combined unless mutually exclusive or otherwise physically impossible.

Further, the techniques, methods, processes, operations, steps, etc. described or suggested herein or in the references incorporated herein, and any methods of using the systems, assemblies, apparatuses, devices, etc. herein, can be performed on a living subject (e.g., human, other animal, etc.) or on a simulation (e.g., a cadaver, cadaver heart, simulator, imaginary person, etc.). When performed on a simulation, the body parts, e.g., heart, tissue, valve, etc., can be assumed to be simulated or can optionally be referred to as “simulated” (e.g., simulated heart, simulated tissue, simulated valve, etc.) and can optionally comprise computerized and/or physical representations of body parts, tissue, etc. The term “simulation” covers use on a cadaver, computer simulator, imaginary person (e.g., if they are just demonstrating in the air on an imaginary heart), etc.

As described herein, when one or more components are described as being connected, joined, affixed, coupled, attached, or otherwise interconnected, such interconnection can be direct as between the components or can be indirect such as through the use of one or more intermediary components. Also as described herein, reference to a “member,” “component,” or “portion” shall not be limited to a single structural member, component, or element but can include an assembly of components, members, or elements. Also as described herein, the terms “substantially” and “about” are defined as at least close to (and includes) a given value or state (preferably within 10% of, more preferably within 1% of, and most preferably within 0.1% of). The terms “clasp” and “clasp arm” are often used herein with respect to specific examples, but the terms “gripping member” and/or “gripper arm” can be used in place of and function in the same or similar ways, even if not configured in the same way as a typical clasp.

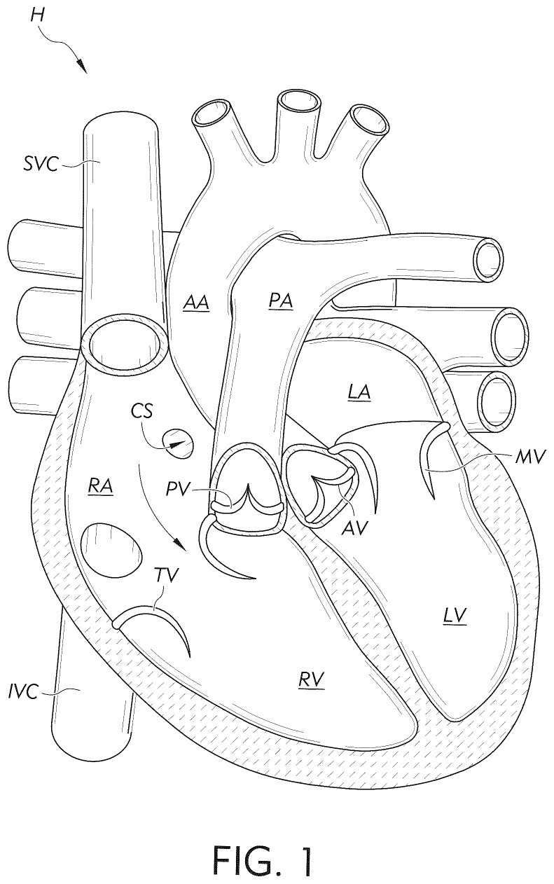

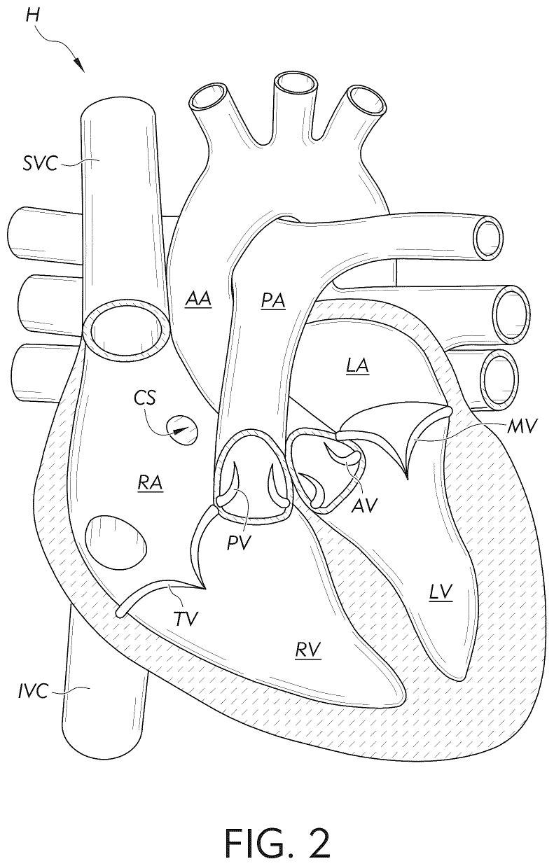

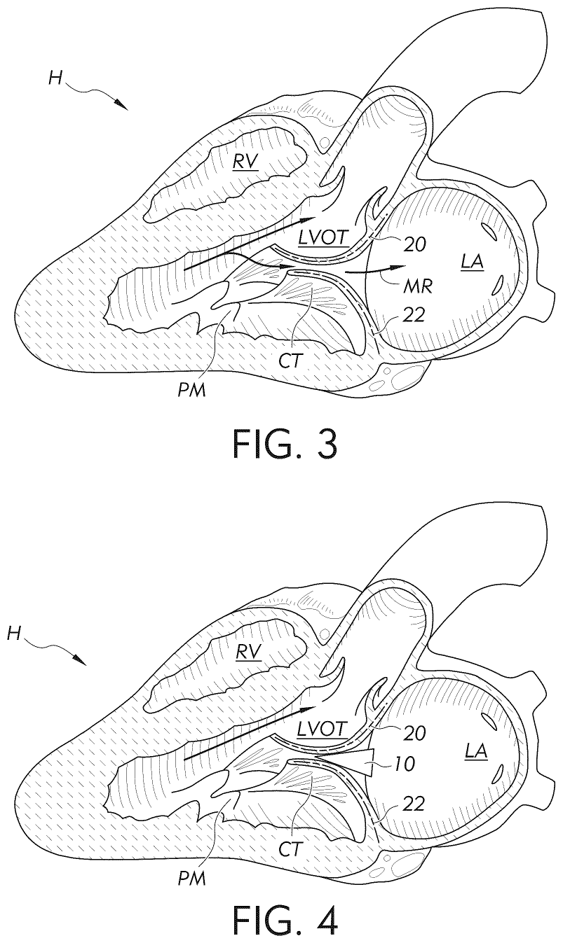

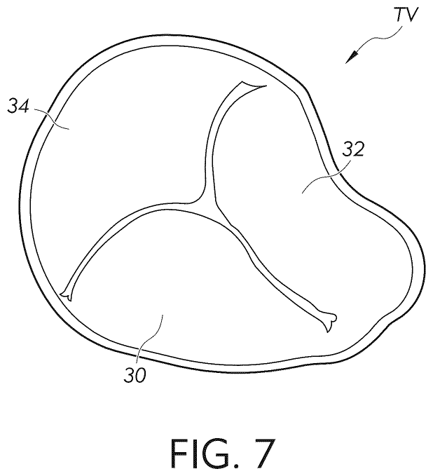

FIGS. 1 and 2 are cutaway views of the human heart H in diastolic and systolic phases, respectively. The right ventricle RV and left ventricle LV are separated from the right atrium RA and left atrium LA, respectively, by the tricuspid valve TV and mitral valve MV; i.e., the atrioventricular valves. Additionally, the aortic valve AV separates the left ventricle LV from the ascending aorta AA, and the pulmonary valve PV separates the right ventricle from the pulmonary artery PA. Each of these valves has flexible leaflets (e.g., leaflets 20, 22 shown in FIGS. 3-6 and leaflets 30, 32, 34 shown in FIG. 7) extending inward across the respective orifices that come together or “coapt” in the flow stream to form the one-way, fluid-occluding surfaces. The native valve repair systems of the present application are frequently described and/or illustrated with respect to the mitral valve MV. Therefore, anatomical structures of the left atrium LA and left ventricle LV will be explained in greater detail. However, the devices described herein can also be used in repairing other native valves, e.g., the devices can be used in repairing the tricuspid valve TV, the aortic valve AV, and the pulmonary valve PV.

The left atrium LA receives oxygenated blood from the lungs. During the diastolic phase, or diastole, seen in FIG. 1, the blood that was previously collected in the left atrium LA (during the systolic phase) moves through the mitral valve MV and into the left ventricle LV by expansion of the left ventricle LV. In the systolic phase, or systole, seen in FIG. 2, the left ventricle LV contracts to force the blood through the aortic valve AV and ascending aorta AA into the body. During systole, the leaflets of the mitral valve MV close to prevent the blood from regurgitating from the left ventricle LV and back into the left atrium LA and blood is collected in the left atrium from the pulmonary vein. In some implementations, the devices described by the present application are used to repair the function of a defective mitral valve MV. That is, the devices are configured to help close the leaflets of the mitral valve to prevent, inhibit or reduce blood from regurgitating from the left ventricle LV and back into the left atrium LA. Many of the devices described in the present application are designed to easily grasp and secure the native leaflets around a coaptation element or spacer that beneficially acts as a filler in the regurgitant orifice to prevent or inhibit back flow or regurgitation during systole, though this is not necessary.

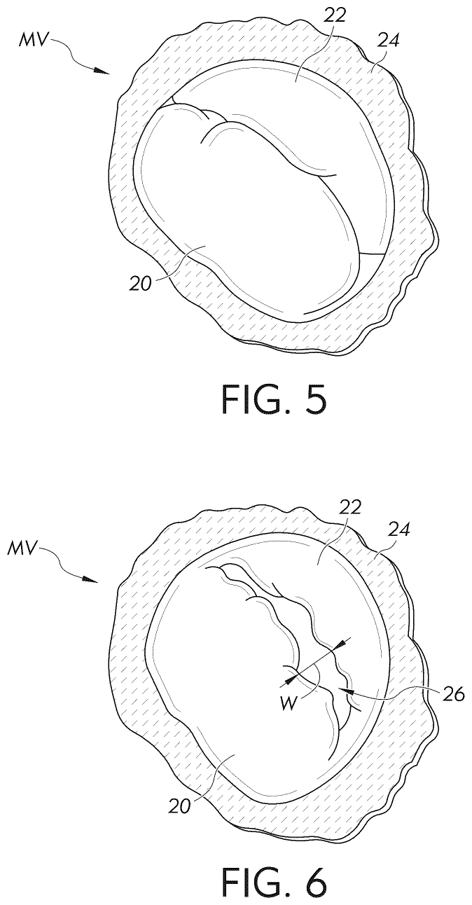

Referring now to FIGS. 1-7, the mitral valve MV includes two leaflets, the anterior leaflet 20 and the posterior leaflet 22. The mitral valve MV also includes an annulus 24 (see FIG. 5), which is a variably dense fibrous ring of tissues that encircles the leaflets 20, 22. Referring to FIGS. 3 and 4, the mitral valve MV is anchored to the wall of the left ventricle LV by chordae tendineae CT. The chordae tendineae CT are cord-like tendons that connect the papillary muscles PM (i.e., the muscles located at the base of the chordae tendineae CT and within the walls of the left ventricle LV) to the leaflets 20, 22 of the mitral valve MV. The papillary muscles PM serve to limit the movements of leaflets 20, 22 of the mitral valve MV and prevent the mitral valve MV from being reverted. The mitral valve MV opens and closes in response to pressure changes in the left atrium LA and the left ventricle LV. The papillary muscles PM do not open or close the mitral valve MV. Rather, the papillary muscles PM support or brace the leaflets 20, 22 against the high pressure needed to circulate blood throughout the body. Together the papillary muscles PM and the chordae tendineae CT are known as the subvalvular apparatus, which functions to keep the mitral valve MV from prolapsing into the left atrium LA when the mitral valve closes. As seen from a Left Ventricular Outflow Tract (LVOT) view shown in FIG. 3, the anatomy of the leaflets 20, 22 is such that the inner sides of the leaflets coapt at the free end portions and the leaflets 20, 22 start receding or spreading apart from each other. The leaflets 20, 22 spread apart in the atrial direction, until each leaflet meets with the mitral annulus.

Various disease processes can impair proper function of one or more of the native valves of the heart H. These disease processes include degenerative processes (e.g., Barlow's Disease, fibroelastic deficiency, etc.), inflammatory processes (e.g., Rheumatic Heart Disease), and infectious processes (e.g., endocarditis, etc.). In addition, damage to the left ventricle LV or the right ventricle RV from prior heart attacks (i.e., myocardial infarction secondary to coronary artery disease) or other heart diseases (e.g., cardiomyopathy, etc.) may distort a native valve's geometry, which may cause the native valve to dysfunction. However, the majority of patients undergoing valve surgery, such as surgery to the mitral valve MV, suffer from a degenerative disease that causes a malfunction in a leaflet (e.g., leaflets 20, 22) of a native valve (e.g., the mitral valve MV), which results in prolapse and regurgitation.

Generally, a native valve may malfunction in different ways: including (1) valve stenosis; and (2) valve regurgitation. Valve stenosis occurs when a native valve does not open completely and thereby causes an obstruction of blood flow. Typically, valve stenosis results from buildup of calcified material on the leaflets of a valve, which causes the leaflets to thicken and impairs the ability of the valve to fully open to permit forward blood flow. Valve regurgitation occurs when the leaflets of the valve do not close completely thereby causing blood to leak back into the prior chamber (e.g., causing blood to leak from the left ventricle to the left atrium).

There are three main mechanisms by which a native valve becomes regurgitant—or incompetent—which include Carpentier's type I, type II, and type III malfunctions. A Carpentier type I malfunction involves the dilation of the annulus such that normally functioning leaflets are distracted from each other and fail to form a tight seal (i.e., the leaflets do not coapt properly). Included in a type I mechanism malfunction are perforations of the leaflets, as are present in endocarditis. A Carpentier's type II malfunction involves prolapse of one or more leaflets of a native valve above a plane of coaptation. A Carpentier's type III malfunction involves restriction of the motion of one or more leaflets of a native valve such that the leaflets are abnormally constrained below the plane of the annulus. Leaflet restriction may be caused by rheumatic disease or dilation of a ventricle.

Referring to FIG. 5, when a healthy mitral valve MV is in a closed position, the anterior leaflet 20 and the posterior leaflet 22 coapt, which prevents blood from leaking from the left ventricle LV to the left atrium LA. Referring to FIGS. 3 and 6, mitral regurgitation MR occurs when the anterior leaflet 20 and/or the posterior leaflet 22 of the mitral valve MV is displaced into the left atrium LA during systole so that the edges of the leaflets 20, 22 are not in contact with each other. This failure to coapt causes a gap 26 between the anterior leaflet 20 and the posterior leaflet 22, which allows blood to flow back into the left atrium LA from the left ventricle LV during systole, as illustrated by the mitral regurgitation MR flow path shown in FIG. 3. Referring to FIG. 6, the gap 26 can have a width W between about 2.5 mm and about 17.5 mm, between about 5 mm and about 15 mm, between about 7.5 mm and about 12.5 mm, or about 10 mm. In some situations, the gap 26 can have a width W greater than 15 mm or even 17.5 mm. As set forth above, there are several different ways that a leaflet (e.g., leaflets 20, 22 of mitral valve MV) may malfunction which may thereby lead to valvular regurgitation.

In any of the above-mentioned situations, a valve repair device or implant is desired that is capable of engaging the anterior leaflet 20 and the posterior leaflet 22 to close the gap 26 and prevent or inhibit regurgitation of blood through the mitral valve MV. As can be seen in FIG. 4, an abstract representation of a valve repair device, an implantable device, or implant 10 is shown implanted between the leaflets 20, 22 such that regurgitation does not occur during systole (compare FIG. 3 with FIG. 4). In some implementations, the coaptation element (e.g., spacer, coaption element, gap filler, membrane, sheet, plug, wedge, balloon, etc.) of the device 10 has a generally tapered or triangular shape that naturally adapts to the native valve geometry and to its expanding leaflet nature (toward the annulus). In this application, the terms spacer, coaption element, coaptation element, gap filler, plug, etc. are used interchangeably and refer to an element that fills a portion of the space between native valve leaflets and/or that is configured such that the native valve leaflets engage or “coapt” against (e.g., such that the native leaflets coapt against the coaption element, coaptation element, spacer, etc. instead of only against one another).

Although stenosis or regurgitation may affect any valve, stenosis is predominantly found to affect either the aortic valve AV or the pulmonary valve PV, and regurgitation is predominantly found to affect either the mitral valve MV or the tricuspid valve TV. Both valve stenosis and valve regurgitation increase the workload of the heart H and may lead to very serious conditions if left un-treated; such as endocarditis, congestive heart failure, permanent heart damage, cardiac arrest, and ultimately death. Because the left side of the heart (i.e., the left atrium LA, the left ventricle LV, the mitral valve MV, and the aortic valve AV) are primarily responsible for circulating the flow of blood throughout the body. Accordingly, because of the substantially higher pressures on the left side heart dysfunction of the mitral valve MV or the aortic valve AV is particularly problematic and often life threatening.

Malfunctioning native heart valves can either be repaired or replaced. Repair typically involves the preservation and correction of the patient's native valve. Replacement typically involves replacing the patient's native valve with a biological or mechanical substitute. Typically, the aortic valve AV and pulmonary valve PV are more prone to stenosis. Because stenotic damage sustained by the leaflets is irreversible, treatments for a stenotic aortic valve or stenotic pulmonary valve can be removal and replacement of the valve with a surgically implanted heart valve, or displacement of the valve with a transcatheter heart valve. The mitral valve MV and the tricuspid valve TV are more prone to deformation of leaflets and/or surrounding tissue, which, as described above, may prevent the mitral valve MV or tricuspid valve TV from closing properly and allows for regurgitation or back flow of blood from the ventricle into the atrium (e.g., a deformed mitral valve MV may allow for regurgitation or back flow from the left ventricle LV to the left atrium LA as shown in FIG. 3). The regurgitation or back flow of blood from the ventricle to the atrium results in valvular insufficiency. Deformations in the structure or shape of the mitral valve MV or the tricuspid valve TV are often repairable. In addition, regurgitation may occur due to the chordae tendineae CT becoming dysfunctional (e.g., the chordae tendineae CT may stretch or rupture), which allows the anterior leaflet 20 and the posterior leaflet 22 to be reverted such that blood is regurgitated into the left atrium LA. The problems occurring due to dysfunctional chordae tendineae CT can be repaired by repairing the chordae tendineae CT or the structure of the mitral valve MV (e.g., by securing the leaflets 20, 22 at the affected portion of the mitral valve).

The devices and procedures disclosed herein often make reference to repairing the structure of a mitral valve. However, it should be understood that the devices and concepts provided herein can be used to repair any native valve, as well as any component of a native valve. Such devices can be used between the leaflets 20, 22 of the mitral valve MV to prevent or inhibit regurgitation of blood from the left ventricle into the left atrium. With respect to the tricuspid valve TV (FIG. 7), any of the devices and concepts herein can be used between any two of the anterior leaflet 30, septal leaflet 32, and posterior leaflet 34 to prevent or inhibit regurgitation of blood from the right ventricle into the right atrium. In addition, any of the devices and concepts provided herein can be used on all three of the leaflets 30, 32, 34 together to prevent or inhibit regurgitation of blood from the right ventricle to the right atrium. That is, the valve repair devices or implants provided herein can be centrally located between the three leaflets 30, 32, 34.

An example device or implant can optionally have a coaptation element (e.g., spacer, coaption element, gap filler, membrane, sheet, plug, wedge, balloon, etc.) and at least one anchor (e.g., one, two, three, or more). In some implementations, an implantable device or implant can have any combination or sub-combination of the features disclosed herein without a coaptation element. When included, the coaptation element (e.g., spacer, coaption element, gap filler, membrane, sheet, plug, wedge, balloon, etc.) is configured to be positioned within the native heart valve orifice to help fill the space between the leaflets and form a more effective seal, thereby reducing or preventing or inhibiting regurgitation described above. The coaptation element can have a structure that is impervious to blood (or that resists blood flow therethrough) and that allows the native leaflets to close around the coaptation element during ventricular systole to block blood from flowing from the left or right ventricle back into the left or right atrium, respectively. The device or implant can be configured to seal against two or three native valve leaflets; that is, the device can be used in the native mitral (bicuspid) and tricuspid valves. The coaptation element is sometimes referred to herein as a spacer because the coaptation element can fill a space between improperly functioning native leaflets (e.g., mitral leaflets 20, 22 or tricuspid leaflets 30, 32, 34) that do not close completely.

The optional coaptation element (e.g., spacer, coaptation element, gap filler, membrane, sheet, plug, wedge, balloon, etc.) can have various shapes. In some implementations, the coaptation element can have an elongated cylindrical shape having a round cross-sectional shape. In some implementations, the coaptation element can have an oval cross-sectional shape, an ovoid cross-sectional shape, a crescent cross-sectional shape, a rectangular cross-sectional shape, or various other non-cylindrical shapes. In some implementations, the coaptation element can have an atrial portion positioned in or adjacent to the atrium, a ventricular or lower portion positioned in or adjacent to the ventricle, and a side surface that extends between the native leaflets. In some implementations configured for use in the tricuspid valve, the atrial or upper portion is positioned in or adjacent to the right atrium, and the ventricular or lower portion is positioned in or adjacent to the right ventricle, and the side surfaces extend between the native tricuspid leaflets.

In some implementations, the anchor can be configured to secure the device to one or both of the native leaflets such that the coaptation element is positioned between the two native leaflets. In some implementations configured for use in the tricuspid valve, the anchor is configured to secure the device to one, two, or three of the tricuspid leaflets such that the coaptation element is positioned between the three native leaflets. In some implementations, the anchor can attach to the coaptation element at a location adjacent the ventricular portion of the coaptation element. In some implementations, the anchor can attach to an actuation element (e.g., an actuation shaft, actuation tube, actuation wire, etc.) to which the coaptation element is also attached. In some implementations, the anchor and the coaptation element can be positioned independently with respect to each other by separately moving each of the anchor and the coaptation element along the longitudinal axis of the actuation element (e.g., actuation shaft, actuation rod, actuation tube, actuation wire, etc.). In some implementations, the anchor and the coaptation element can be positioned simultaneously by moving the anchor and the coaptation element together along the longitudinal axis of the actuation element (e.g., shaft, actuation wire, etc.). The anchor can be configured to be positioned behind a native leaflet when implanted such that the leaflet is grasped by the anchor.

The device or implant can be configured to be implanted via a delivery system or other means for delivery. The delivery system can comprise one or more of a guide/delivery sheath, a delivery catheter, a steerable catheter, an implant catheter, tube, combinations of these, etc. The coaptation element and the anchor can be compressible to a radially compressed state and can be self-expandable to a radially expanded state when compressive pressure is released. The device can be configured for the anchor to be expanded radially away from the still compressed coaptation element initially in order to create a gap between the coaptation element and the anchor. A native leaflet can then be positioned in the gap. The coaptation element can be expanded radially, closing the gap between the coaptation element and the anchor and capturing the leaflet between the coaptation element and the anchor. In some implementations, the anchor and coaptation element are optionally configured to self-expand. The implantation methods for various implementations can be different and are more fully discussed below with respect to each implementation. Additional information regarding these and other delivery methods can be found in U.S. Pat. No. 8,449,599 and U.S. Patent Application Publication Nos. 2014/0222136, 2014/0067052, 2016/0331523, and PCT patent application publication Nos. WO2020/076898, each of which is incorporated herein by reference in its entirety for all purposes. These method(s) can be performed on a living animal or on a simulation, such as on a cadaver, cadaver heart, simulator (e.g., with the body parts, heart, tissue, etc. being simulated), etc. mutatis mutandis.

The disclosed devices or implants can be configured such that the anchor is connected to a leaflet, taking advantage of the tension from native chordae tendineae to resist high systolic pressure urging the device toward the left atrium. During diastole, the devices can rely on the compressive and retention forces exerted on the leaflet that is grasped by the anchor.

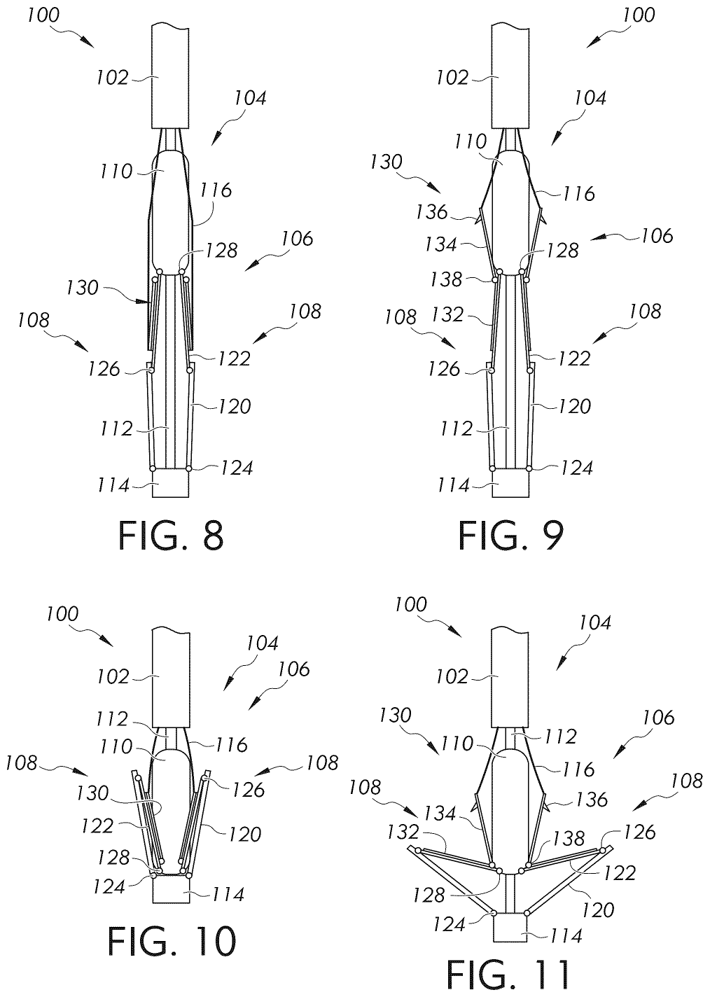

Referring now to FIGS. 8-15, a schematically illustrated device or implant 100 (e.g., a prosthetic device, a valve repair device, implantable device, etc.) is shown in various stages of deployment. The device or implant 100 and other similar devices/implants are described in more detail in PCT patent application publication Nos. WO2018/195215, WO2020/076898, and WO 2019/139904, which are incorporated herein by reference in their entirety. The device 100 can include any other features for another device or implant discussed in the present application or the applications cited above, and the device 100 can be positioned to engage valve tissue (e.g., leaflets 20, 22, 30, 32, 34) as part of any suitable valve repair system (e.g., any valve repair system disclosed in the present application or the applications cited above).

The device or implant 100 is deployed from a delivery system 102. The delivery system 102 can comprise one or more of a catheter, a sheath, a guide catheter/sheath, a delivery catheter/sheath, a steerable catheter, an implant catheter, a tube, a channel, a pathway, combinations of these, etc. The device or implant 100 includes a coaptation portion 104 and an anchor portion 106.

In some implementations, the coaptation portion 104 of the device or implant 100 includes a coaptation element 110 that is adapted to be implanted between leaflets of a native valve (e.g., a native mitral valve, native tricuspid valve, etc.) and is slidably attached to an actuation element 112 (e.g., actuation wire, shaft, tube, hypotube, line, suture, braid, etc.). The anchor portion 106 includes one or more anchors 108 that are actuatable between open and closed conditions and can take a wide variety of forms, such as, for example, paddles, gripping elements, or the like. Actuation of the actuation element 112 opens and closes the anchor portion 106 of the device 100 to grasp the native valve leaflets during implantation. The actuation element 112 (as well as other actuation elements disclosed herein) can take a wide variety of different forms (e.g., as a wire, rod, shaft, tube, screw, suture, line, strip, combination of these, etc.), be made of a variety of different materials, and have a variety of configurations. As one example, the actuation element can be threaded such that rotation of the actuation element moves the anchor portion 106 relative to the coaptation portion 104. Or, the actuation element can be unthreaded, such that pushing or pulling the actuation element 112 moves the anchor portion 106 relative to the coaptation portion 104.

The anchor portion 106 and/or anchors of the device 100 include outer paddles 120 and inner paddles 122 that are, in some implementations, connected between a cap 114 and a coaptation element 110 by portions 124, 126, 128. The portions 124, 126, 128 can be jointed and/or flexible to move between all of the positions described below. The interconnection of the outer paddles 120, the inner paddles 122, the coaptation element 110, and the cap 114 by the portions 124, 126, and 128 can constrain the device to the positions and movements illustrated herein.

In some implementations, the delivery system 102 includes a steerable catheter, implant catheter, and the actuation element 112 (e.g., actuation wire, shaft, tube, hypotube, line, suture, braid, etc.). These can be configured to extend through a guide catheter/sheath (e.g., a transseptal sheath, etc.). In some implementations, the actuation element 112 extends through a delivery catheter and the coaptation element 110 to the distal end (e.g., a cap 114 or other attachment portion at the distal connection of the anchor portion 106). Extending and retracting the actuation element 112 increases and decreases the spacing between the coaptation element 110 and the distal end of the device (e.g., the cap 114 or other attachment portion), respectively. In some implementations, a collar or other attachment element (e.g., clamp, clip, lock, sutures, friction fit, buckle, snap fit, lasso, etc.) removably attaches the coaptation element 110 to the delivery system 102, either directly or indirectly, so that the actuation element 112 slides through the collar or other attachment element and, in some implementations, through a coaptation element 110 during actuation to open and close the paddles 120, 122 of the anchor portion 106 and/or anchors 108.

In some implementations, the anchor portion 106 and/or anchors 108 can include attachment portions or gripping members (e.g., gripping arms, clasp arms, etc.). The illustrated gripping members can comprise clasps 130 that include a base or fixed arm 132, a moveable arm 134, optional friction-enhancing elements, other securing structures 136 (e.g., barbs, protrusions, ridges, grooves, textured surfaces, adhesive, etc.), and a joint portion 138. The fixed arms 132 are attached to the inner paddles 122. In some implementations, the fixed arms 132 are attached to the inner paddles 122 with the joint portion 138 disposed proximate the coaptation element 110. The joint portion 138 provides a spring force between the fixed and moveable arms 132, 134 of the clasp 130. The joint portion 138 can be any suitable joint, such as a flexible joint, a spring joint, a pivot joint, or the like. In some implementations, the joint portion 138 is a flexible piece of material integrally formed with the fixed and moveable arms 132, 134. The fixed arms 132 are attached to the inner paddles 122 and remain stationary or substantially stationary relative to the inner paddles 122 when the moveable arms 134 are opened to open the clasps 130 and expose the optional barbs or other friction-enhancing elements 136.

In some implementations, the clasps 130 are opened by applying tension to actuation lines 116 attached to the moveable arms 134, thereby causing the moveable arms 134 to articulate, flex, or pivot on the joint portions 138. The actuation lines 116 extend through the delivery system 102 (e.g., through a steerable catheter and/or an implant catheter). Other actuation mechanisms are also possible.

The actuation line 116 can take a wide variety of forms, such as, for example, a line, a suture, a wire, a rod, a catheter, or the like. The clasps 130 can be spring loaded so that in the closed position the clasps 130 continue to provide a pinching force on the grasped native leaflet. Optional barbs or other friction-enhancing elements 136 of the clasps 130 can grab, pinch, and/or pierce the native leaflets to further secure the native leaflets.

During implantation, the paddles 120, 122 can be opened and closed, for example, to grasp the native leaflets (e.g., native mitral valve leaflets, etc.) between the paddles 120, 122 and/or between the paddles 120, 122 and a coaptation element 110 (e.g., a spacer, plug, membrane, etc.). The clasps 130 can be used to grasp and/or further secure the native leaflets by engaging the leaflets with optional barbs or other friction-enhancing elements 136 and pinching the leaflets between the moveable and fixed arms 134, 132. The optional barbs or other friction-enhancing elements 136 (e.g., protrusions, ridges, grooves, textured surfaces, adhesive, etc.) of the clasps 130 increase friction with the leaflets or can partially or completely puncture the leaflets. The actuation lines 116 can be actuated separately so that each clasp 130 can be opened and closed separately. Separate operation allows one leaflet to be grasped at a time, or for the repositioning of a clasp 130 on a leaflet that was insufficiently grasped, without altering a successful grasp on the other leaflet. The clasps 130 can be opened and closed relative to the position of the inner paddle 122 (as long as the inner paddle is in an open or at least partially open position), thereby allowing leaflets to be grasped in a variety of positions as the particular situation requires.

Referring now to FIG. 8, the device 100 is shown in an elongated or fully open condition for deployment from an implant delivery catheter of the delivery system 102. The device 100 is disposed at the end of the catheter of the delivery system 102 in the fully open position. In the elongated condition the cap 114 is spaced apart from the coaptation element 110 such that the paddles 120, 122 are fully extended. In some implementations, an angle formed between the interior of the outer and inner paddles 120, 122 is approximately 180 degrees. The clasps 130 can be kept in a closed condition during deployment through the delivery system. The actuation lines 116 can extend and attach to the moveable arms 134.

Referring now to FIG. 9, the device 100 is shown in an elongated condition, similar to FIG. 8, but with the clasps 130 in a fully open position, ranging from about 140 degrees to about 200 degrees, from about 170 degrees to about 190 degrees, or about 180 degrees between fixed and moveable arms 132, 134 of the clasps 130.

Referring now to FIG. 10, the device 100 is shown in a shortened or fully closed condition. To move the device 100 from the elongated condition to the shortened condition, the actuation element 112 is retracted to pull the cap 114 towards the coaptation element 110. The connection portion(s) 126 (e.g., joint(s), flexible connection(s), etc.) between the outer paddle 120 and inner paddle 122 are constrained in movement such that compression forces acting on the outer paddle 120 from the cap 114 being retracted towards the coaptation element 110 cause the paddles or gripping elements to move radially outward. During movement from the open position to the closed position, the outer paddles 120 maintain an acute angle with the actuation element 112. The outer paddles 120 can optionally be biased toward a closed position. The inner paddles 122 during the same motion move through a considerably larger angle as they are oriented away from the coaptation element 110 in the open condition and collapse along the sides of the coaptation element 110 in the closed condition.

Referring now to FIGS. 11-13, the device 100 is shown in a partially open, grasp-ready condition. To transition from the fully closed to the partially open condition, the actuation element (e.g., actuation wire, shaft, tube, hypotube, line, suture, braid, etc.) is extended to push the cap 114 away from the coaptation element 110, thereby pulling on the outer paddles 120, which in turn pull on the inner paddles 122, causing the anchors or anchor portion 106 to partially unfold. The actuation lines 116 are also retracted to open the clasps 130 so that the leaflets can be grasped. In some implementations, the pair of inner and outer paddles 122, 120 are moved in unison, rather than independently, by a single actuation element 112. Also, the positions of the clasps 130 are dependent on the positions of the paddles 122, 120. For example, referring to FIG. 10 closing the paddles 122, 120 also closes the clasps. In some implementations, the paddles 120, 122 can be independently controllable. In the example illustrated by FIG. 15, the device 100 can have two actuation elements 111, 113 and two independent caps 115, 117 (or other attachment portions), such that one independent actuation element (e.g., actuation wire, shaft, tube, hypotube, line, suture, braid, etc.) and cap (or other attachment portion) are used to control one paddle, and the other independent actuation element and cap (or other attachment portion) are used to control the other paddle.

Referring now to FIG. 12, one of the actuation lines 116 is extended to allow one of the clasps 130 to close. Referring now to FIG. 13, the other actuation line 116 is extended to allow the other clasp 130 to close. Either or both of the actuation lines 116 can be repeatedly actuated to repeatedly open and close the clasps 130.

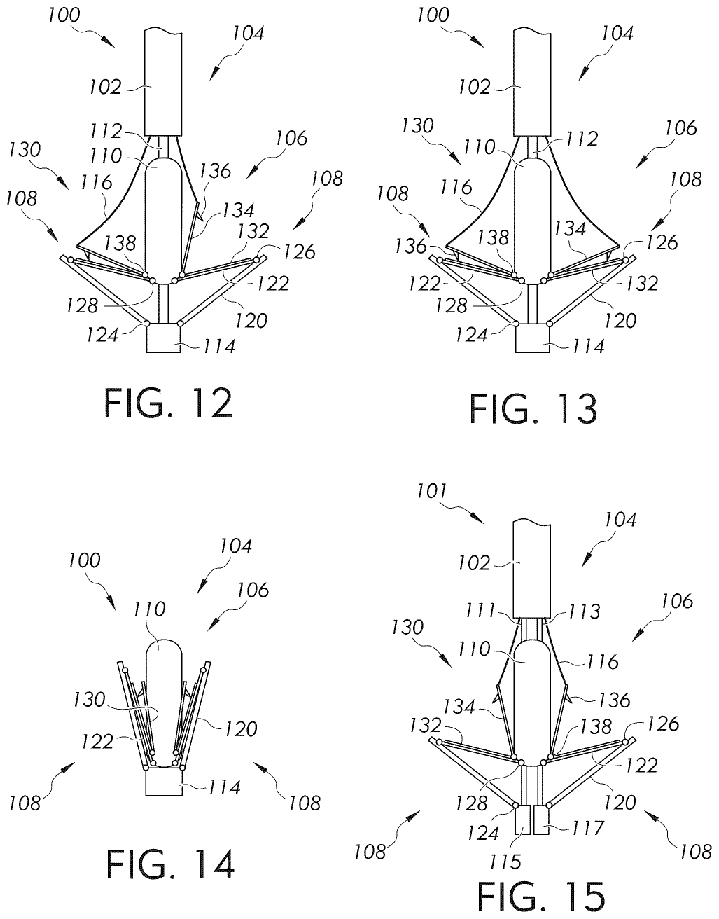

Referring now to FIG. 14, the device 100 is shown in a fully closed and deployed condition. The delivery system 102 and actuation element 112 are retracted and the paddles 120, 122 and clasps 130 remain in a fully closed position. Once deployed, the device 100 can be maintained in the fully closed position with a mechanical latch or can be biased to remain closed through the use of spring materials, such as steel, other metals, plastics, composites, etc. or shape-memory alloys such as Nitinol. For example, the connection portions 124, 126, 128, the joint portions 138, and/or the inner and outer paddles 122, and/or an additional biasing component (not shown) can be formed of metals such as steel or shape-memory alloy, such as Nitinol—produced in a wire, sheet, tubing, or laser sintered powder—and are biased to hold the outer paddles 120 closed around the coaptation element 110 and the clasps 130 pinched around native leaflets. Similarly, the fixed and moveable arms 132, 134 of the clasps 130 are biased to pinch the leaflets.

In some implementations, the attachment or connection portions 124, 126, 128, joint portions 138, and/or the inner and outer paddles 122, and/or an additional biasing component (not shown) can be formed of any other suitably elastic material, such as a metal or polymer material, to maintain the device 100 in the closed condition after implantation.

FIG. 15 illustrates an example where the paddles 120, 122 are independently controllable. The device 101 illustrated by FIG. 15 is similar to the device illustrated by FIG. 11, except the device 100 of FIG. 15 includes an actuation element that is configured as two independent actuation elements 111, 113 that are coupled to two independent caps 115, 117. To transition a first inner paddle 122 and a first outer paddle 120 from the fully closed to the partially open condition, the actuation element 111 is extended to push the cap 115 away from the coaptation element 110, thereby pulling on the outer paddle 120, which in turn pulls on the inner paddle 122, causing the first anchor 108 to partially unfold. To transition a second inner paddle 122 and a second outer paddle 120 from the fully closed to the partially open condition, the actuation element 113 is extended to push the cap 115 away from the spacer or coaptation element 110, thereby pulling on the outer paddle 120, which in turn pulls on the inner paddle 122, causing the second anchor 108 to partially unfold. The independent paddle control illustrated by FIG. 15 can be implemented on any of the devices disclosed by the present application. For comparison, in the example illustrated by FIG. 11, the pair of inner and outer paddles 122, 120 are moved in unison, rather than independently, by a single actuation element 112.

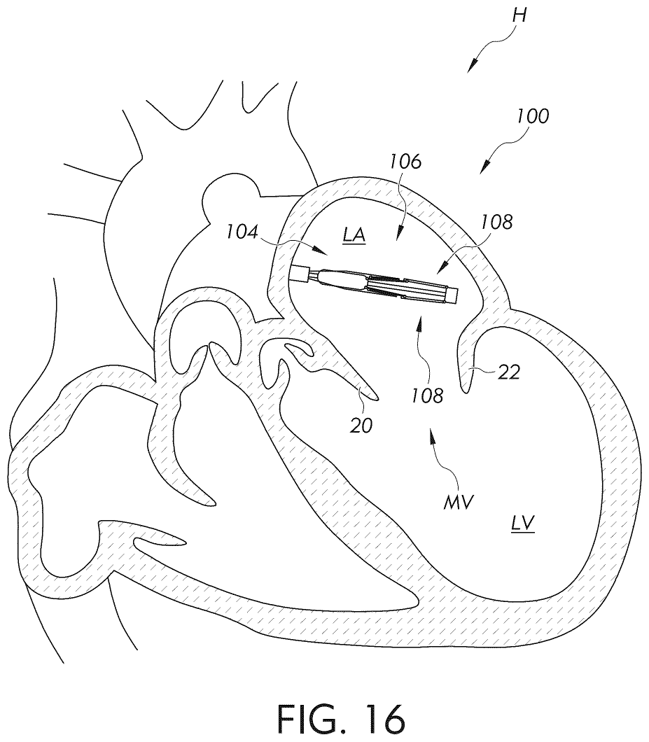

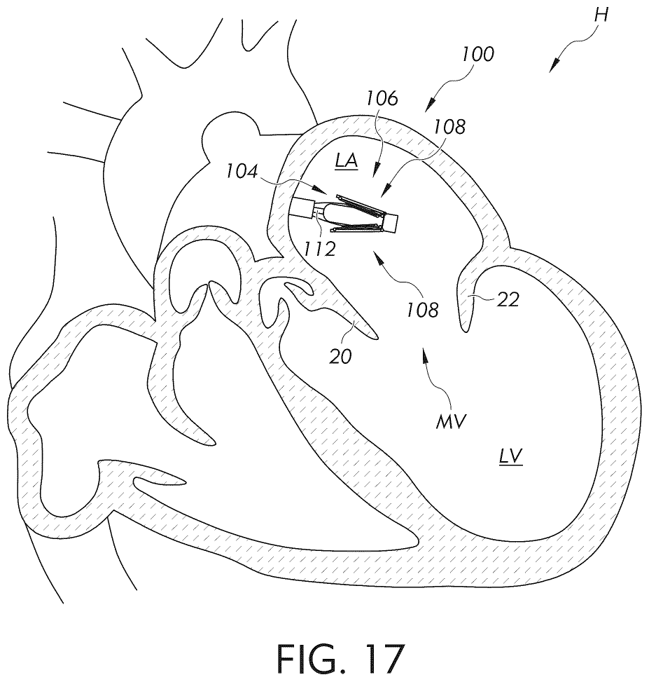

Referring now to FIGS. 16-21, the device 100 of FIGS. 8-14 is shown being delivered and deployed within the native mitral valve MV of the heart H. Referring to FIG. 16, a delivery sheath/catheter is inserted into the left atrium LA through the septum and the implant/device 100 is deployed from the delivery catheter/sheath in the fully open condition as illustrated in FIG. 16. The actuation element 112 is then retracted to move the implant/device into the fully closed condition shown in FIG. 17.

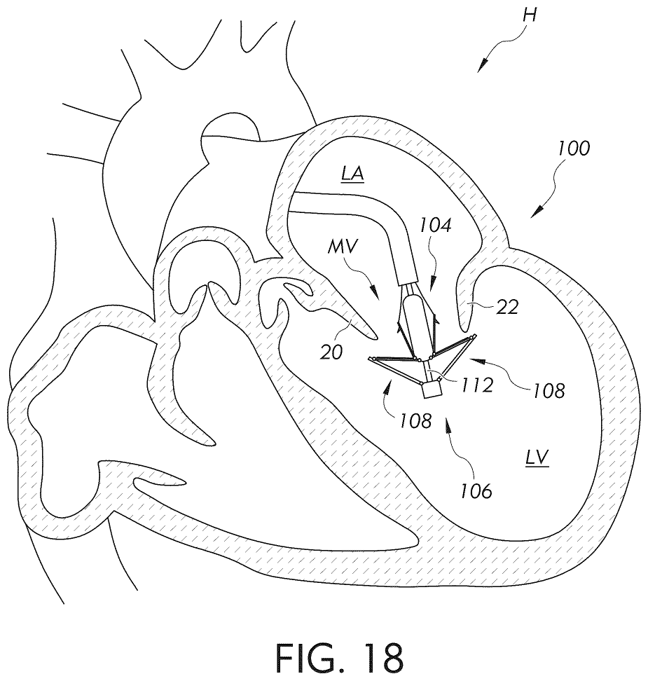

As can be seen in FIG. 18, the implant/device is moved into position within the mitral valve MV into the ventricle LV and partially opened so that the leaflets 20, 22 can be grasped. For example, a steerable catheter can be advanced and steered or flexed to position the steerable catheter as illustrated by FIG. 18. The implant catheter connected to the implant/device can be advanced from inside the steerable catheter to position the implant as illustrated by FIG. 18.

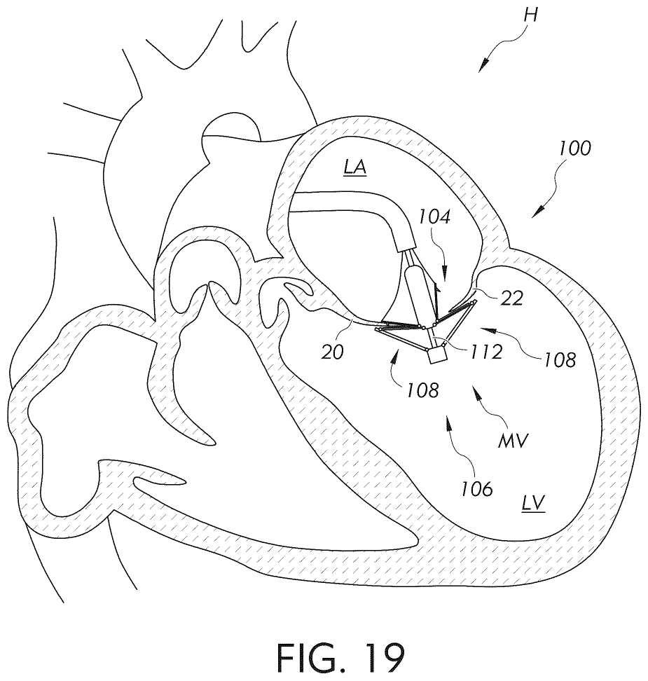

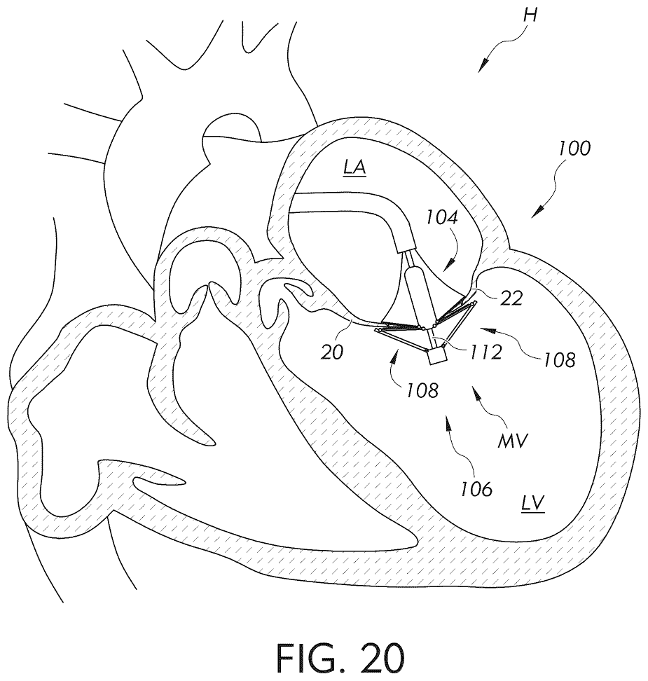

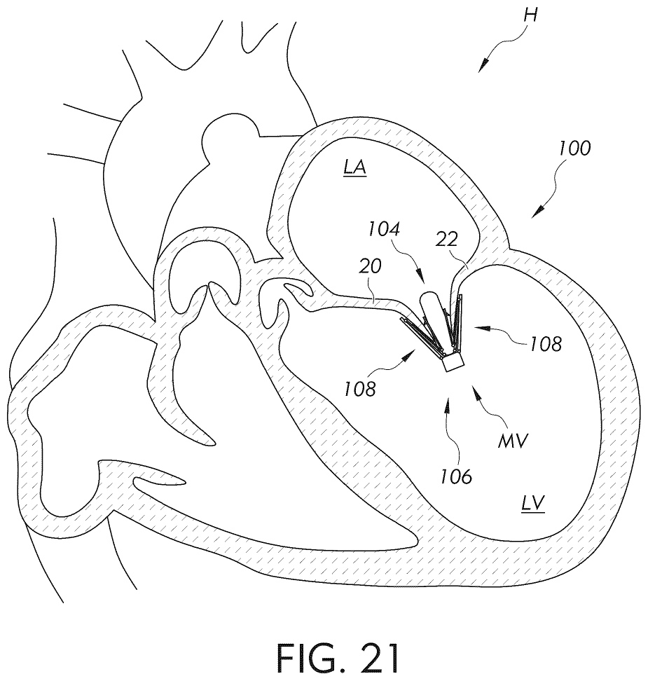

Referring now to FIG. 19, the implant catheter can be retracted into the steerable catheter to position the mitral valve leaflets 20, 22 in the clasps 130. An actuation line 116 is extended to close one of the clasps 130, capturing a leaflet 20. FIG. 20 shows the other actuation line 116 being then extended to close the other clasp 130, capturing the remaining leaflet 22. Lastly, as can be seen in FIG. 21, the delivery system 102 (e.g., steerable catheter, implant catheter, etc.), actuation element 112 and actuation lines 116 are then retracted and the device or implant 100 is fully closed and deployed in the native mitral valve MV.

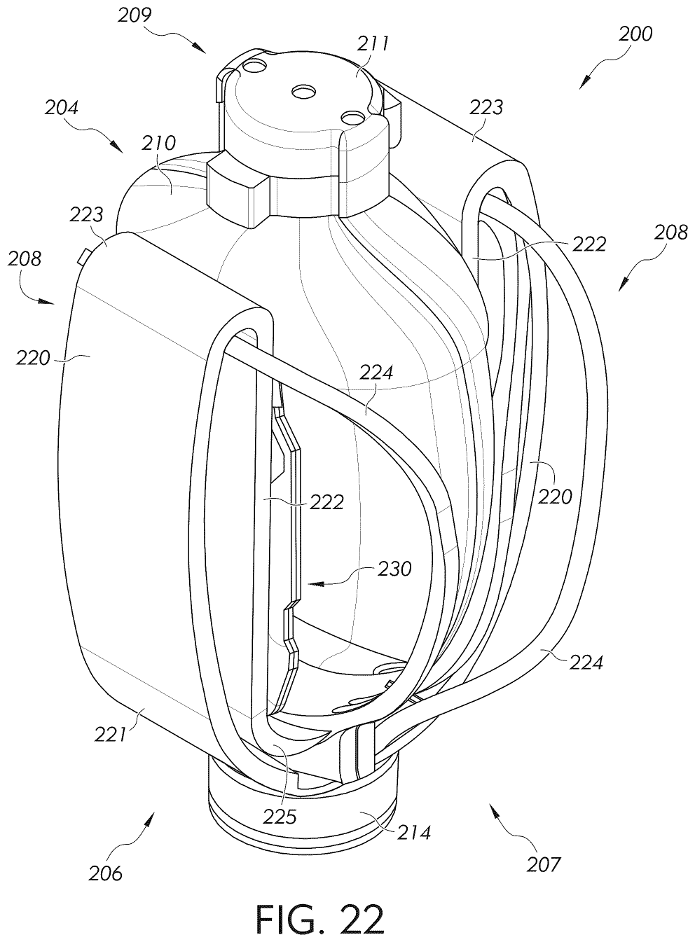

Any of the features disclosed by the present application can be used in a wide variety of different valve repair devices. FIGS. 22-24 illustrate examples of valve repair devices that can be modified to include any of the features disclosed by the present application. Any combination or sub-combination of the features disclosed by the present application can be combined with, substituted for, and/or added to any combination or sub-combination of the features of the valve repair devices illustrated by FIGS. 8-24.

Referring now to FIG. 22, an example of an implantable device or implant 200 is shown. The device 200 is one of the many different configurations that the device 100 that is schematically illustrated in FIGS. 8-14 can take. The device 200 can include any other features for an implantable device or implant discussed in the present application, and the device 200 can be positioned to engage valve tissue 20, 22 as part of any suitable valve repair system (e.g., any valve repair system disclosed in the present application). The device/implant 200 can be a prosthetic spacer device, valve repair device, or another type of implant that attaches to leaflets of a native valve.

In some implementations, the implantable device or implant 200 includes a coaptation portion 204, a proximal or attachment portion 209, an anchor portion 206, and a distal portion 207. In some implementations, the coaptation portion 204 of the device optionally includes a coaptation element 210 (e.g., spacer, coaptation element, gap filler, membrane, sheet, plug, wedge, balloon, etc.) for implantation between leaflets of a native valve. In some implementations, the anchor portion 206 includes a plurality of anchors 208.

The anchors can be configured in a variety of ways. In some implementations, each anchor 208 includes outer paddles 220, inner paddles 222, paddle extension members or paddle frames 224, and clasps 230. In some implementations, the attachment portion 209 includes a first or proximal collar 211 (or other attachment element) for engaging with a capture mechanism of a delivery system. A delivery system for the device 200 can be the same as or similar to delivery system 102 described above and can comprise one or more of a catheter, a sheath, a guide catheter/sheath, a delivery catheter/sheath, a steerable catheter, an implant catheter, a tube, a channel, a pathway, combinations of these, etc. The capture mechanism can be configured in a variety of ways and, in some implementations, can comprise one or more of a clamp, clip, pin, suture, line, lasso, noose, snare, buckle, lock, latch, etc.

In some implementations, the coaptation element 210 and paddles 220, 222 are formed from a flexible material that can be a metal fabric, such as a mesh, woven, braided, or formed in any other suitable way or a laser cut or otherwise cut flexible material. The material can be cloth, shape-memory alloy wire—such as Nitinol—to provide shape-setting capability, or any other flexible material suitable for implantation in the human body.

An actuation element (e.g., actuation wire, shaft, tube, hypotube, line, suture, braid, etc.) can extend from a delivery system (not shown) to engage and enable actuation of the device or implant 200. In some implementations, the actuation element extends through the proximal collar 211, and spacer or coaptation element 210 to engage a cap 214 of the distal portion 207. The actuation element can be configured to removably engage the cap 214 with a threaded connection, or the like, so that the actuation element can be disengaged and removed from the device 200 after implantation.

The coaptation element 210 extends from the proximal collar 211 (or other attachment element) to the inner paddles 222. In some implementations, the coaptation element 210 has a generally elongated and round shape, though other shapes and configurations are possible. In some implementations, the coaptation element 210 has an elliptical shape or cross-section when viewed from above and has a tapered shape or cross-section when seen from a front view and a round shape or cross-section when seen from a side view. A blend of these three geometries can result in the three-dimensional shape of the illustrated coaptation element 210 that achieves the benefits described herein. The round shape of the coaptation element 210 can also be seen, when viewed from above, to substantially follow or be close to the shape of the paddle frames 224.

The size and/or shape of the coaptation element 210 can be selected to minimize the number of implants that a single patient will require (preferably one), while at the same time maintaining low transvalvular gradients. In some implementations, the anterior-posterior distance at the top of the coaptation element is about 5 mm, and the medial-lateral distance of the coaptation element at its widest is about 10 mm. In some implementations, the overall geometry of the device 200 can be based on these two dimensions and the overall shape strategy described above. It should be readily apparent that the use of other anterior-posterior distance anterior-posterior distance and medial-lateral distance as starting points for the device will result in a device having different dimensions. Further, using other dimensions and the shape strategy described above will also result in a device having different dimensions.

In some implementations, the outer paddles 220 are jointably attached to the cap 214 of the distal portion 207 by connection portions 221 and to the inner paddles 222 by connection portions 223. The inner paddles 222 are jointably attached to the coaptation element by connection portions 225. In this manner, the anchors 208 are configured similar to legs in that the inner paddles 222 are like upper portions of the legs, the outer paddles 220 are like lower portions of the legs, and the connection portions 223 are like knee portions of the legs.

In some implementations, the inner paddles 222 are stiff, relatively stiff, rigid, have rigid portions and/or are stiffened by a stiffening member or a fixed portion of the clasps 230. The inner paddle 222, the outer paddle 220, and the coaptation element can all be interconnected as described herein.

In some implementations, the paddle frames 224 are attached to the cap 214 at the distal portion 207 and extend to the connection portions 223 between the inner and outer paddles 222, 220. In some implementations, the paddle frames 224 are formed of a material that is more rigid and stiff than the material forming the paddles 222, 220 so that the paddle frames 224 provide support for the paddles 222, 220.

The paddle frames 224 can provide additional pinching force between the inner paddles 222 and the coaptation element 210 and assist in wrapping the leaflets around the sides of the coaptation element 210. That is, the paddle frames 224 can be configured with a round three-dimensional shape extending from the cap 214 to the connection portions 223 of the anchors 208. The connections between the paddle frames 224, the outer and inner paddles 220, 222, the cap 214, and the coaptation element 210 can constrain each of these parts to the movements and positions described herein. In particular the connection portion 223 is constrained by its connection between the outer and inner paddles 220, 222 and by its connection to the paddle frame 224. Similarly, the paddle frame 224 is constrained by its attachment to the connection portion 223 (and thus the inner and outer paddles 222, 220) and to the cap 214.

The wide configuration of the paddle frames 224 provides increased surface area compared to the inner paddles 222 alone. The increased surface area can distribute the clamping force of the paddles 220 and paddle frames 224 against the native leaflets over a relatively larger surface of the native leaflets in order to further protect the native leaflet tissue.

Additional features of the device 200, modified versions of the device, delivery systems for the device, and methods for using the device and delivery system are disclosed by Patent Cooperation Treaty International Application No. PCT/US2018/028189 (International Publication No. WO 2018/195215). Any combination or sub-combination of the features disclosed by the present application can be combined with any combination or sub-combination of the features disclosed by Patent Cooperation Treaty International Application No. PCT/US2018/028189 (International Publication No. WO 2018/195215). Patent Cooperation Treaty International Application No. PCT/US2018/028189 (International Publication No. WO 2018/195215) is incorporated herein by reference in its entirety.

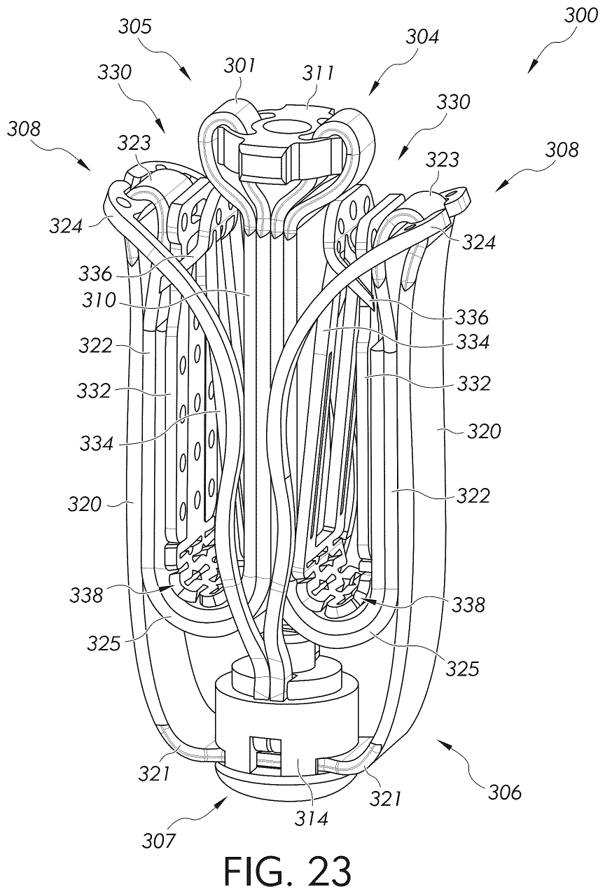

Referring now to FIG. 23, an example of a device or implant 300 is shown. The device 300 is one of the many different configurations that the device 100 that is schematically illustrated in FIGS. 8-14 can take. The device 300 can include any other features for a device or implant discussed in the present application, and the device 300 can be positioned to engage valve tissue 20, 22 as part of any suitable valve repair system (e.g., any valve repair system disclosed in the present application).

The device or implant 300 includes a proximal or attachment portion 305, an anchor portion 306, and a distal portion 307. In some implementations, the device/implant 300 includes a coaptation portion 304, and the coaptation portion 304 can optionally include a coaptation element 310 (e.g., spacer, plug, membrane, sheet, etc.) for implantation between the leaflets 20, 22 of the native valve. In some implementations, the anchor portion 306 includes a plurality of anchors 308. In some implementations, each anchor 308 can include one or more paddles, e.g., outer paddles 320, inner paddles 322, paddle extension members or paddle frames 324. The anchors can also include and/or be coupled to clasps 330. In some implementations, the attachment portion 305 includes a first or proximal collar 311 (or other attachment element) for engaging with a capture mechanism of a delivery system.

The anchors 308 can be attached to the other portions of the device and/or to each other in a variety of different ways (e.g., directly, indirectly, welding, sutures, adhesive, links, latches, integrally formed, a combination of some or all of these, etc.). In some implementations, the anchors 308 are attached to a coaptation element 310 by connection portions 325 and to a cap 314 by connection portions 321.

The anchors 308 can comprise first portions or outer paddles 320 and second portions or inner paddles 322 separated by connection portions 323. The connection portions 323 can be attached to paddle frames 324 that are hingeably attached to a cap 314 or other attachment portion. In this manner, the anchors 308 are configured similar to legs in that the inner paddles 322 are like upper portions of the legs, the outer paddles 320 are like lower portions of the legs, and the connection portions 323 are like knee portions of the legs.

In implementations with a coaptation element 310, the coaptation element 310 and the anchors 308 can be coupled together in various ways. As shown in the illustrated example, the coaptation element 310 and the anchors 308 can be coupled together by integrally forming the coaptation element 310 and the anchors 308 as a single, unitary component. This can be accomplished, for example, by forming the coaptation element 310 and the anchors 308 from a continuous strip 301 of a braided or woven material, such as braided or woven nitinol wire. In the illustrated example, the coaptation element 310, the outer paddle portions 320, the inner paddle portions 322, and the connection portions 321, 323, 325 are formed from a continuous strip of fabric 301.

Like the anchors 208 of the device or implant 200 described above, the anchors 308 can be configured to move between various configurations by axially moving the distal end of the device (e.g., cap 314, etc.) relative to the proximal end of the device (e.g., proximal collar 311 or other attachment element, etc.). This movement can be along a longitudinal axis extending between the distal end (e.g., cap 314, etc.) and the proximal end (e.g., collar 311 or other attachment element, etc.) of the device.

In some implementations, in the straight configuration, the paddle portions 320, 322 are aligned or straight in the direction of the longitudinal axis of the device. In some implementations, the connection portions 323 of the anchors 308 are adjacent the longitudinal axis of the spacer or coaptation element 310. From the straight configuration, the anchors 308 can be moved to a fully folded configuration (e.g., FIG. 23), e.g., by moving the proximal end and distal end toward each other and/or toward a midpoint or center of the device.

In some implementations, the clasps comprise a moveable arm coupled to an anchor. In some implementations, the clasps 330 include a base or fixed arm 332, a moveable arm 334, optional barbs/friction-enhancing elements 336, and a joint portion 338. The fixed arms 332 are attached to the inner paddles 322, with the joint portion 338 disposed proximate the coaptation element 310. The joint portion 338 is spring-loaded so that the fixed and moveable arms 332, 334 are biased toward each other when the clasp 330 is in a closed condition.

The fixed arms 332 are attached to the inner paddles 322 through holes or slots with sutures. The fixed arms 332 can be attached to the inner paddles 322 with any suitable means, such as screws or other fasteners, crimped sleeves, mechanical latches or snaps, welding, adhesive, or the like. The fixed arms 332 remain substantially stationary relative to the inner paddles 322 when the moveable arms 334 are opened to open the clasps 330 and expose optional the optional barbs 336. The clasps 330 are opened by applying tension to actuation lines attached to the moveable arms 334, thereby causing the moveable arms 334 to articulate, pivot, and/or flex on the joint portions 338.

In short, the device or implant 300 is similar in configuration and operation to the device or implant 200 described above, except that the coaptation element 310, outer paddles 320, inner paddles 322, and connection portions 321, 323, 325 are formed from the single strip of material 301. In some implementations, the strip of material 301 is attached to the proximal collar 311, cap 314, and paddle frames 324 by being woven or inserted through openings in the proximal collar 311, cap 314, and paddle frames 324 that are configured to receive the continuous strip of material 301. The continuous strip 301 can be a single layer of material or can include two or more layers. In some implementations, portions of the device 300 have a single layer of the strip of material 301 and other portions are formed from multiple overlapping or overlying layers of the strip of material 301.

For example, FIG. 23 shows a coaptation element 310 and inner paddles 322 formed from multiple overlapping layers of the strip of material 301. The single continuous strip of material 301 can start and end in various locations of the device 300. The ends of the strip of material 301 can be in the same location or different locations of the device 300. For example, in the illustrated example of FIG. 23, the strip of material 301 begins and ends in the location of the inner paddles 322.

As with the device or implant 200 described above, the size of the coaptation element 310 can be selected to minimize the number of implants that a single patient will require (preferably one), while at the same time maintaining low transvalvular gradients. In particular, forming many components of the device 300 from the strip of material 301 allows the device 300 to be made smaller than the device 200. For example, in some implementations, the anterior-posterior distance at the top of the coaptation element 310 is less than 2 mm, and the medial-lateral distance of the device 300 (i.e., the width of the paddle frames 324 which are wider than the coaptation element 310) at its widest is about 5 mm.

Additional features of the device 300, modified versions of the device, delivery systems for the device, and methods for using the device and delivery system are disclosed by Patent Cooperation Treaty International Application No. PCT/US2019/055320 (International Publication No. WO 2020/076898). Any combination or sub-combination of the features disclosed by the present application can be combined with any combination or sub-combination of the features disclosed by Patent Cooperation Treaty International Application No. PCT/US2019/055320 (International Publication No. WO 2020/076898). Patent Cooperation Treaty International Application No. PCT/US2019/055320 (International Publication No. WO 2020/076898) is incorporated herein by reference in its entirety.

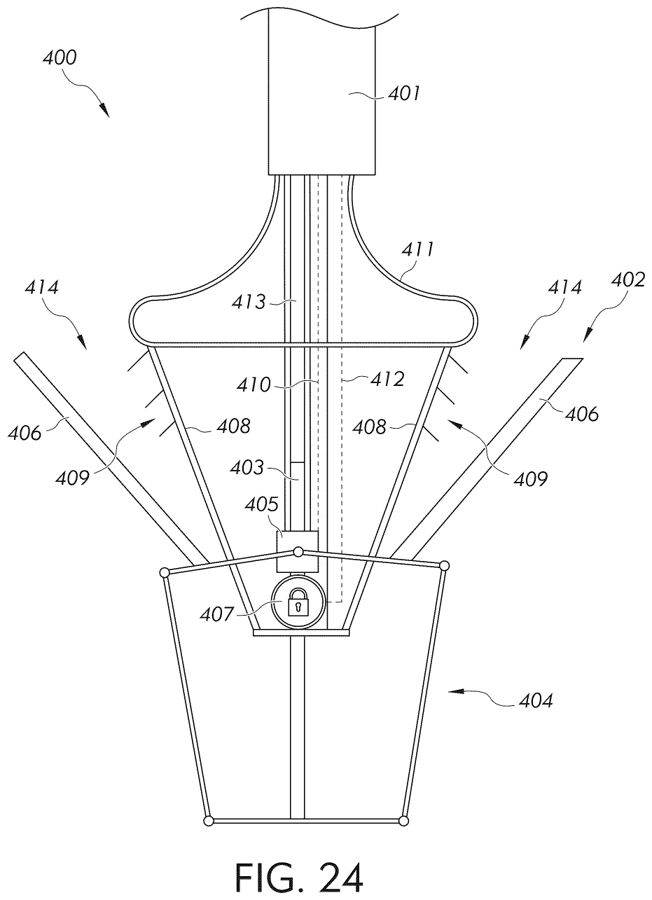

FIG. 24 illustrates an example of one of the many valve repair systems 400 useable for repairing a native valve of a patient that the concepts of the present application can be applied to. The valve repair system 400 includes a delivery device 401 and a valve repair device 402.

The valve repair device 402 includes a base assembly 404, a pair of paddles 406, and a pair of gripping members 408 (e.g., clasps, clasp arms, grippers, gripping arms, latches, etc.). In one example, the paddles 406 can be integrally formed with the base assembly. For example, the paddles 406 can be formed as extensions of links of the base assembly. In the illustrated example, the base assembly 404 of the valve repair device 402 has a shaft 403, a coupler 405 configured to move along the shaft, and a lock 407 configured to lock the coupler in a stationary position on the shaft. The coupler 405 is mechanically connected to the paddles 406, such that movement of the coupler 405 along the shaft 403 causes the paddles to move between an open position and a closed position. In this way, the coupler 405 serves as a means for mechanically coupling the paddles 406 to the shaft 403 and, when moving along the shaft 403, for causing the paddles 406 to move between their open and closed positions.

In some implementations, the gripping members 408 are pivotally connected to the base assembly 404 (e.g., the gripping members 408 can be pivotally connected to the shaft 403, or any other suitable member of the base assembly), such that the gripping members can be moved to adjust the width of the opening 414 between the paddles 406 and the gripping members 408. The gripping member 408 can include an optional barbed portion 409 for attaching the gripping members to valve tissue when the valve repair device 402 is attached to the valve tissue. When the paddles 406 are in the closed position, the paddles engage the gripping members 408, such that, when valve tissue is attached to the barbed portion 409 of the gripping members, the paddles secure the valve repair device 402 to the valve tissue. In some implementations, the gripping members 408 are configured to engage the paddles 406 such that the optional barbed portion 409 engages the valve tissue member and the paddles 406 to secure the valve repair device 402 to the valve tissue member. For example, in certain situations, it can be advantageous to have the paddles 406 maintain an open position and have the gripping members 408 move outward toward the paddles 406 to engage valve tissue and the paddles 406.

While the example shown in FIG. 24 illustrates a pair of paddles 406 and a pair of gripping members 408, it should be understood that the valve repair device 402 can include any suitable number of paddles and gripping members.

In some implementations, the valve repair system 400 includes a placement shaft 413 that is removably attached to the shaft 403 of the base assembly 404 of the valve repair device 402. After the valve repair device 402 is secured to valve tissue, the placement shaft 413 is removed from the shaft 403 to remove the valve repair device 402 from the remainder of the valve repair system 400, such that the valve repair device 402 can remain attached to the valve tissue, and the delivery device 401 can be removed from a patient's body.

The valve repair system 400 can also include a paddle control mechanism 410, a gripper control mechanism 411, and a lock control mechanism 412. The paddle control mechanism 410 is mechanically attached to the coupler 405 to move the coupler along the shaft, which causes the paddles 406 to move between the open and closed positions. The paddle control mechanism 410 can take any suitable form and can comprise, for example, a shaft, wire, tube, hypotube, rod, suture, line, etc. For example, the paddle control mechanism can comprise a hollow shaft, a catheter tube or a sleeve that fits over the placement shaft 413 and the shaft 403 and is connected to the coupler 405.

The gripper control mechanism 411 is configured to move the gripping members 408 such that the width of the opening 414 between the gripping members and the paddles 406 can be altered. The gripper control mechanism 411 can take any suitable form, such as, for example, a line, a suture, a wire, a rod, a catheter, a tube, a hypotube, etc.

The lock control mechanism 412 is configured to lock and unlock the lock. The lock 407 locks the coupler 405 in a stationary position with respect to the shaft 403 and can take a wide variety of different forms and the type of lock control mechanism 412 can be dictated by the type of lock used. In examples in which the lock 407 includes a pivotable plate, the lock control mechanism 412 is configured to engage the pivotable plate to move the plate between the tilted and substantially non-tilted positions. The lock control mechanism 412 can be, for example, a rod, a suture, a wire, or any other member that is capable of moving a pivotable plate of the lock 407 between a tilted and substantially non-tilted position.

The valve repair device 402 is movable from an open position to a closed position. The base assembly 404 includes links that are moved by the coupler 405. The coupler 405 is movably attached to the shaft 403. In order to move the valve repair device from the open position to the closed position, the coupler 405 is moved along the shaft 403, which moves the links.

The gripper control mechanism 411 is moves the gripping members 408 to provide a wider or a narrower gap at the opening 414 between the gripping members and the paddles 406. In the illustrated example, the gripper control mechanism 411 includes a line, such as a suture, a wire, etc. that is connected to an opening in an end of the gripping members 408. When the line(s) is pulled, the gripping members 408 move inward, which causes the opening 414 between the gripping members and the paddles 406 to become wider.

In order to move the valve repair device 402 from the open position to the closed position, the lock 407 is moved to an unlocked condition by the lock control mechanism 412. Once the lock 407 is in the unlocked condition, the coupler 405 can be moved along the shaft 403 by the paddle control mechanism 410.

After the paddles 406 are moved to the closed position, the lock 407 is moved to the locked condition by the locking control mechanism 412 to maintain the valve repair device 402 in the closed position. After the valve repair device 402 is maintained in the locked condition by the lock 407, the valve repair device 402 is removed from the delivery device 401 by disconnecting the shaft 403 from the placement shaft 413. In addition, the valve repair device 402 is disengaged from the paddle control mechanism 410, the gripper control mechanism 411, and the lock control mechanism 412.

Additional features of the device 402, modified versions of the device, delivery systems for the device, and methods for using the device and delivery system are disclosed by Patent Cooperation Treaty International Application No. PCT/US2019/012707 (International Publication No. WO 2019139904). Any combination or sub-combination of the features disclosed by the present application can be combined with any combination or sub-combination of the features disclosed by Patent Cooperation Treaty International Application No. PCT/US2019/012707 (International Publication No. WO 2019139904). Patent Cooperation Treaty International Application No. PCT/US2019/012707 (International Publication No. WO 2019139904) is incorporated herein by reference in its entirety.

Clasps or leaflet gripping devices disclosed herein can take a wide variety of different forms. Examples of clasps are disclosed by Patent Cooperation Treaty International Application No. PCT/US2018/028171 (International Publication No. WO 2018195201). Any combination or sub-combination of the features disclosed by the present application can be combined with any combination or sub-combination of the features disclosed by Patent Cooperation Treaty International Application No. PCT/US2018/028171 (International Publication No. WO 2018195201). Patent Cooperation Treaty International Application No. PCT/US2018/028171 (International Publication No. WO 2018195201) is incorporated herein by reference in its entirety.

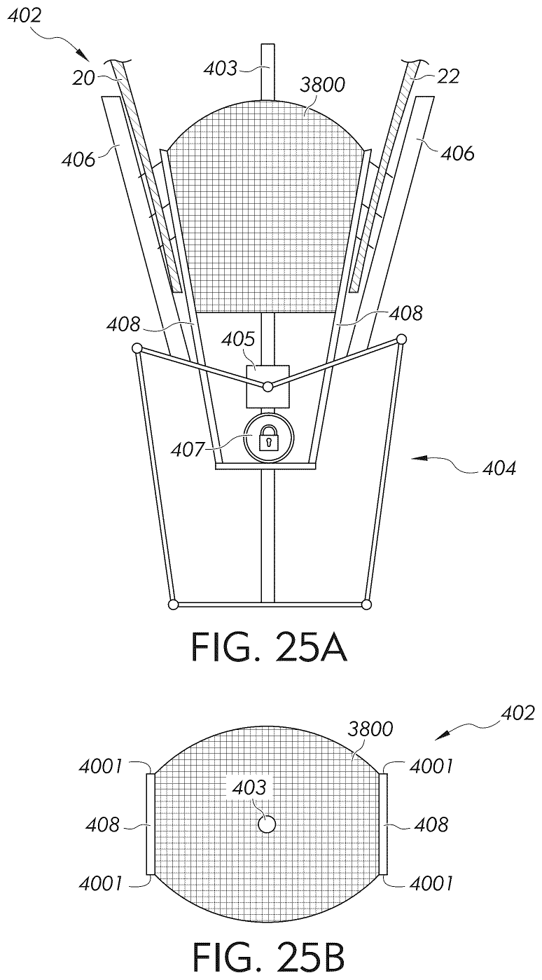

Referring to FIGS. 25A-25B, an example implementation of a valve repair device 402 has a coaptation element 3800. The valve repair device 402 can have the same configuration as the valve repair device illustrated by FIG. 24 with the addition of the coaptation element. The coaptation element 3800 can take a wide variety of different forms. The coaptation element 3800 can be compressible and/or expandable. For example, the coaptation element can be compressed to fit inside one or more catheters of a delivery system, can expand when moved out of the one or more catheters, and/or can be compressed by the paddles 406 to adjust the size of the coaptation element. In the example illustrated by FIGS. 25A and 25B, the size of the coaptation element 3800 can be reduced by squeezing the coaptation element with the paddles 406 and can be increased by moving the paddles 406 away from one another. The coaptation element 3800 can extend past outer edges 4001 of the gripping members or clasps 408 as illustrated for providing additional surface area for closing the gap of a mitral valve.

The coaptation element 3800 can be coupled to the valve repair device 402 in a variety of different ways. For example, the coaptation element 3800 can be fixed to the shaft 403, can be slidably disposed around the shaft, can be connected to the coupler 405, can be connected to the lock 407, and/or can be connected to a central portion of the clasps or gripping members 408. In some implementations, the coupler 405 can take the form of the coaptation element 3800. That is, a single element can be used as the coupler 405 that causes the paddles 406 to move between the open and closed positions and the coaptation element 3800 that closes the gap between the leaflets 20, 22 when the valve repair device 402 is attached to the leaflets.

The coaptation element 3800 can be disposed around one or more of the shafts or other control elements of the valve repair system 400. For example, the coaptation element 3800 can be disposed around the shaft 403, the shaft 413, the paddle control mechanism 410, and/or the lock control mechanism 412.

The valve repair device 402 can include any other features for a valve repair device discussed in the present application, and the valve repair device 402 can be positioned to engage valve tissue as part of any suitable valve repair system (e.g., any valve repair system disclosed in the present application). Additional features of the device 402, modified versions of the device, delivery systems for the device, and methods for using the device and delivery system are disclosed by Patent Cooperation Treaty International Application No. PCT/US2019/012707 (International Publication No. WO 2019139904). Any combination or sub-combination of the features disclosed by the present application can be combined with any combination or sub-combination of the features disclosed by Patent Cooperation Treaty International Application No. PCT/US2019/012707 (International Publication No. WO 2019139904).

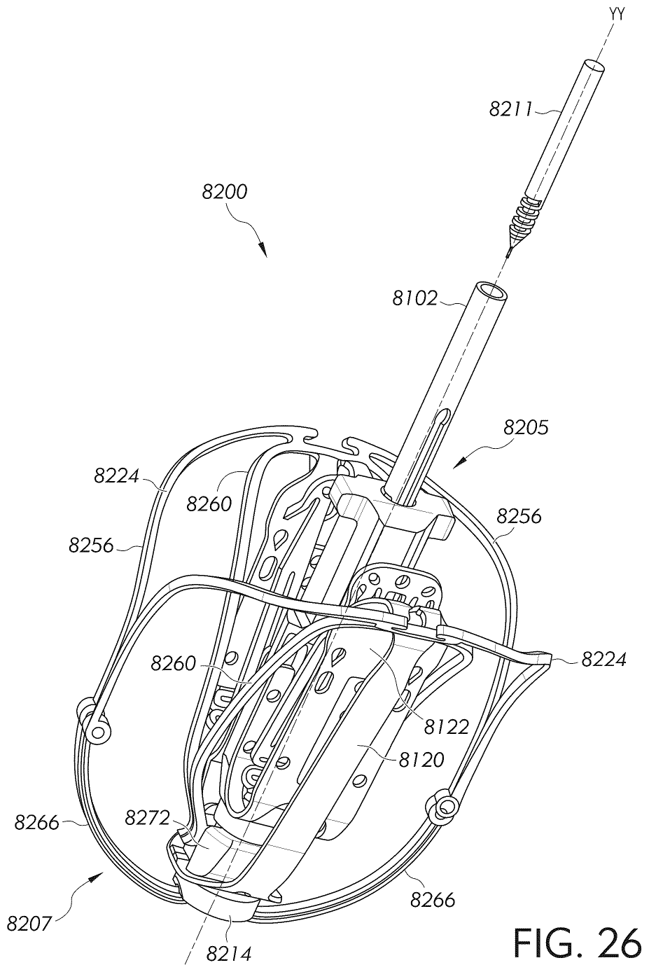

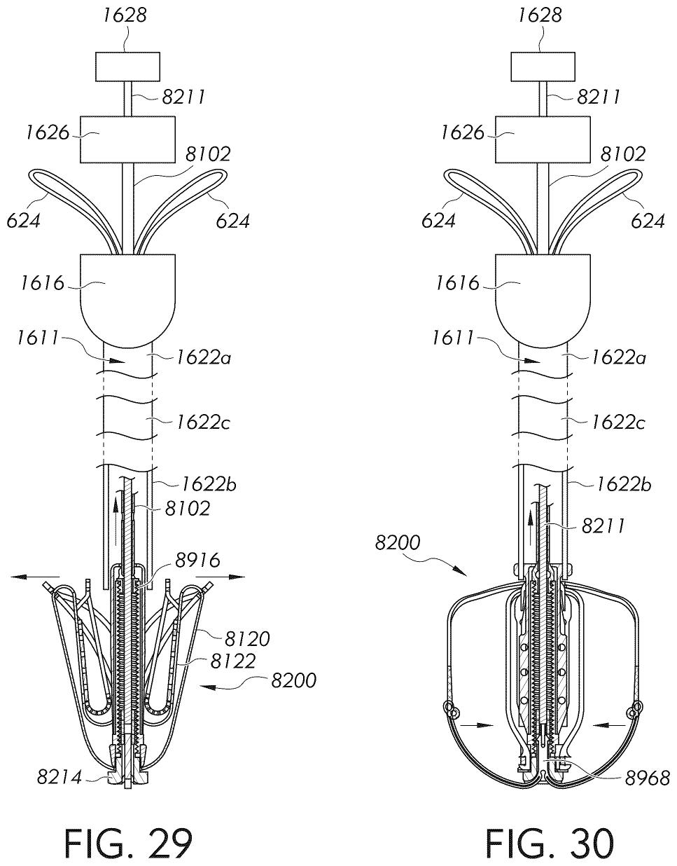

FIGS. 26-30 illustrate an example of one of the many valve repair systems useable for repairing a native valve of a patient that the concepts of the present application can be applied to. Referring to FIGS. 29 and 30, the valve repair system includes an implant catheter assembly 1611 and an implantable valve repair device 8200. Referring to FIGS. 26-28, the device 8200 includes a proximal or attachment portion 8205, paddle frames 8224, inner paddle portions 8122, outer paddle portions 8120, and a distal portion 8207. The attachment portion 8205, the distal portion 8207, and the paddle frames 8224 can be configured in a variety of ways.

In the example illustrated in FIG. 26, the paddle frames 8224 can be symmetric along longitudinal axis YY. However, in some implementations, the paddle frames 8224 are not symmetric about the axis YY. Moreover, referring to FIG. 26, the paddle frames 8224 include outer frame portions 8256 and inner frame portions 8260.

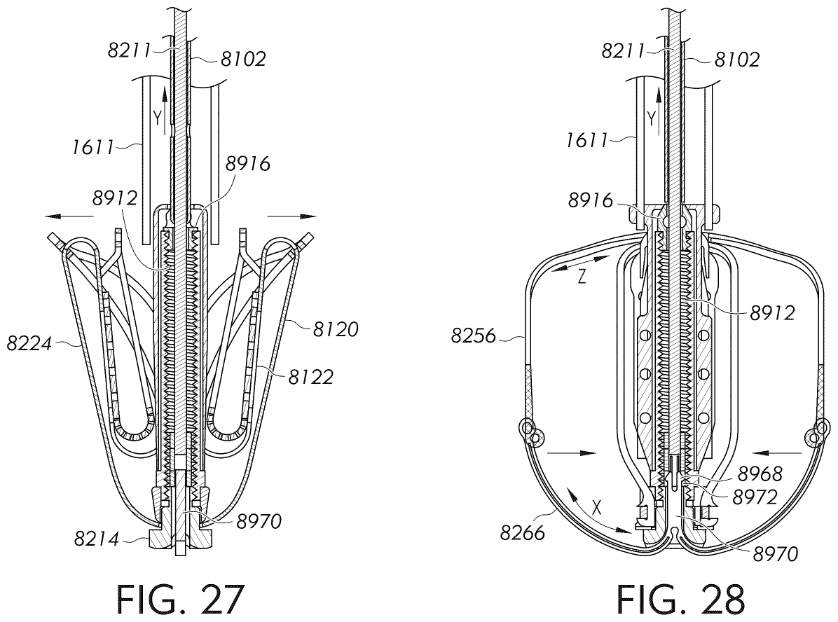

In some implementations, the connector 8266 (e.g., shaped metal component, shaped plastic component, tether, wire, strut, line, cord, suture, etc.) attaches to the outer frame portions 8256 at outer ends of the connector 8266 and to a coupler 8972 at an inner end 8968 of the connector 8266 (see FIG. 28). Between the connector 8266 and the attachment portion 8205, the outer frame portions 8256 form a curved shape. For example, in the illustrated example, the shape of the outer frame portions 8256 resembles an apple shape in which the outer frame portions 8256 are wider toward the attachment portion 8205 and narrower toward the distal portion 8207. In some implementations, however, the outer frame portions 8256 can be otherwise shaped.

The inner frame portions 8260 extend from the attachment portion 8205 toward the distal portion 8207. The inner frame portions 8260 then extend inward to form retaining portions 8272 that are attached to the actuation cap 8214. The retaining portions 8272 and the actuation cap 8214 can be configured to attach in any suitable manner.

In some implementations, the inner frame portions 8260 are rigid frame portions, while the outer frame portions 8256 are flexible frame portions. The proximal end of the outer frame portions 8256 connect to the proximal end of the inner frame portions 8260, as illustrated in FIG. 26.

The width adjustment element 8211 (e.g., width adjustment wire, width adjustment shaft, width adjustment tube, width adjustment line, width adjustment cord, width adjustment suture, width adjustment screw or bolt, etc.) is configured to move the outer frame portions 8256 from the expanded position to the narrowed position by pulling the inner end 8968 (FIG. 28) and portions of the connector 8266 into the actuation cap 8214. The actuation element 8102 is configured to move the inner frame portions 8260 to open and close the paddles in accordance with some implementations disclosed herein.

As shown in FIGS. 27 and 28, the connector 8266 has an inner end 8968 that engages with the width adjustment element 8211 such that a user can move the inner end 8968 inside the receiver 8912 (e.g., an internally threaded element, a column, a conduit, a hollow member, a notched receiving portion, a tube, a shaft, a sleeve, a post, a housing, a cylinder, tracks, etc.) to move the outer frame portions 8256 between a narrowed position and an expanded position. In the illustrated example, the inner end 8968 includes a post 8970 that attaches to the outer frame portions 8256 and a coupler 8972 that extends from the post 8970. The coupler 8972 is configured to attach and detach from both the width adjustment element 8211 and the receiver 8912.

The coupler 8972 can take a wide variety of different forms. For example, the coupler 8972 can include one or more of a threaded connection, features that mate with threads, detent connections, such as outwardly biased arms, walls, or other portions. When the coupler 8972 is attached to the width adjustment element 8211, the coupler is released from the receiver 8912. When the coupler 8972 is detached from the width adjustment element 8211, the coupler is secured to the receiver. The inner end 8968 of the connector can, however, be configured in a variety of ways. Any configuration that can suitably attach the outer frame portions 8256 to the coupler to allow the width adjustment element 8211 to move the outer frame portions 8256 between the narrowed position and the expanded position can be used. The coupler can be configured in a variety of ways as well and can be a separate component or be integral with another portion of the device, e.g., of the connector or inner end of the connector.

The width adjustment element 8211 allows a user to expand or contract the outer frame portions 8256 of the device 8200. In the example illustrated in FIGS. 27 and 28, the width adjustment element 8211 includes an externally threaded end that is threaded into the coupler 8972. The width adjustment element 8211 moves the coupler in the receiver 8912 to adjust the width of the outer frame portions 8256. When the width adjustment element 8211 is unscrewed from the coupler 8972, the coupler engages the inner surface of the receiver 8912 to set the width of the outer frame portions 8256.

In some implementations, the receiver 8912 can be integrally formed with a distal cap 8214. Moving the cap 8214 relative to a body of the attachment portion 8205 opens and closes the paddles. In the illustrated example, the receiver 8912 slides inside the body of the attachment portion. When the coupler 8972 is detached from the width adjustment element 8211, the width of the outer frame portions 8256 is fixed while the actuation element 8102 moves the receiver 8912 and cap 8214 relative to a body of the attachment portion 8205. Movement of the cap can open and close the device in the same manner as the other implementations disclosed above.

In the illustrated example, a driver head 8916 is disposed at a proximal end of the actuation element 8102. The driver head 8916 releasably couples the actuation element 8102 to the receiver 8912. In the illustrated example, the width adjustment element 8211 extends through the actuation element 8102. The actuation element is axially advanced in the direction opposite to direction Y to move the distal cap 8214. Movement of the distal cap 8214 relative to the attachment portion 8205 is effective to open and close the paddles, as indicated by the arrows in FIG. 27. That is, movement of the distal cap 8214 in the direction Y closes the device and movement of the distal cap in the direction opposite to direction Y opens the device.

Also illustrated in FIGS. 27 and 28, the width adjustment element 8211 extends through the actuation element 8102, the driver head 8916, and the receiver 8912 to engage the coupler 8972 attached to the inner end 8968. The movement of the outer frame portions 8256 to the narrowed position can allow the device or implant 8200 to maneuver more easily into position for implantation in the heart by reducing the contact and/or friction between the native structures of the heart—e.g., chordae—and the device 8200. The movement of the outer frame portions 8256 to the expanded position provides the anchor portion of the device or implant 8200 with a larger surface area to engage and capture leaflet(s) of a native heart valve.

Referring to FIGS. 29 and 30, an implementation of an implant catheter assembly 1611 in which clasp actuation lines 624 extend through a handle 1616, the actuation element 8102 is coupled to a paddle actuation control 1626, and the width adjustment element 8211 is coupled to a paddle width control 1628. A proximal end portion 1622a of the shaft or catheter of the implant catheter assembly 1611 can be coupled to the handle 1616, and a distal end portion 1622b of the shaft or catheter can be coupled to the device 8200. The actuation element 8102 can extend distally from the paddle actuation control 1626, through the handle 1616, through the delivery shaft or catheter of the implant catheter assembly 1611, and through the proximal end of the device 8200, where it couples with the driver head 8916. The actuation element 8102 can be axially movable relative to the outer shaft of the implant catheter assembly 1611 and the handle 1616 to open and close the device.

The width adjustment element 8211 can extend distally from the paddle width control 1628, through the paddle actuation control 1626 and through the actuation element 8102 (and, consequently, through the handle 1616, the outer shaft of the implant catheter assembly 1611, and through the device 8200), where it couples with the movable coupler 8972. The width adjustment element 8211 can be axially movable relative to the actuation element 8102, the outer shaft of the implant catheter assembly 1611, and the handle 1616. The clasp actuation lines 624 can extend through and be axially movable relative to the handle 1616 and the outer shaft of the implant catheter assembly 1611. The clasp actuation lines 624 can also be axially movable relative to the actuation element 8102.

Referring to FIGS. 29 and 30, the width adjustment element 8211 can be releasably coupled to the coupler 8972 of the device 8200. Advancing and retracting the width adjustment element 8211 with the paddle width control 1628 widens and narrows the paddles. Advancing and retracting the actuation element 8102 with the paddle actuation control 1626 opens and closes the paddles of the device.

In the examples of FIGS. 29 and 30, the catheter or shaft of the implant catheter assembly 1611 is an elongate shaft extending axially between the proximal end portion 1622a, which is coupled to the handle 1616, and the distal end portion 1622b, which is coupled to the device 8200. The outer shaft of the implant catheter assembly 1611 can also include an intermediate portion 1622c disposed between the proximal and distal end portions 1622a, 1622b.

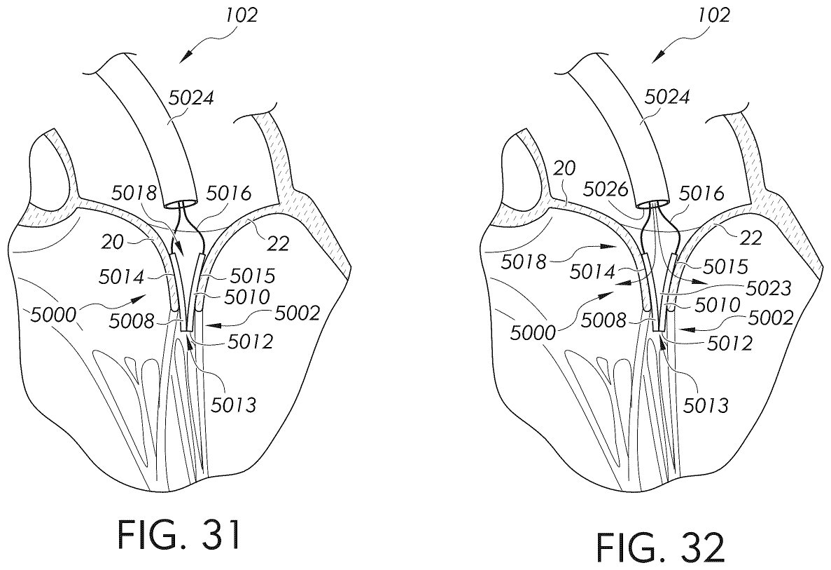

FIGS. 31-34 schematically illustrate a method and a device or implant 5000 (e.g., a prosthetic device, a valve repair device, implantable device, etc.) useable for repairing a native heart valve (e.g., the mitral valve or the tricuspid valve). The device or implant 5000 can include any of the features for another device or implant discussed in the present application or the applications cited above, and the device or implant 5000 can be positioned to engage valve tissue (e.g., leaflets 20, 22, 30, 32, 34) as part of any suitable valve repair system (e.g., any valve repair system disclosed in the present application, or the applications cited herein).

The device or implant 5000 is deployed from a delivery system 102. The delivery system 102 can comprise one or more of a catheter, a sheath, a guide catheter/sheath, a delivery catheter/sheath, a steerable catheter, an implant catheter, a tube, a channel, a pathway, combinations of these, etc. The device or implant 5000 includes a leaflet coaptation assist element 5002, one or more fixation elements 5004, and a lock 5006.

The leaflet coaptation assist element 5002 is adapted to block or inhibit regurgitant flow and/or suppress and/or prevent the native leaflets (20, 22, 30, 32, 34) from prolapsing or flailing, thereby restoring coaptation in the case of valve regurgitation. The coaptation assist element 5002 can be configured in a variety of ways. Any configuration that can block or inhibit regurgitant flow and/or suppress and/or prevent the native leaflets (20, 22, 30, 32, 34) from prolapsing or flailing can be used. In some implementations, the coaptation assist element 5002 is configured to be implanted between leaflets 20, 22, 30, 32, 34 of a native valve (e.g., a native mitral valve, native tricuspid valve, etc.) to prevent or inhibit the leaflets from prolapsing. The size and shape of the coaptation assist element 5002 can vary. In some implementations, the coaptation assist element 5002 has a first delivery state (e.g., narrow state) suitable for delivery through a catheter and a second deployed state (e.g., expanded or wider state) suitable for blocking or inhibiting regurgitant flow and/or inhibiting the leaflets from prolapsing.

In the illustrated example of FIGS. 31-34, the leaflet coaptation assist element 5002 is positioned between the anterior leaflet 20 and the posterior leaflet 22 of the mitral valve MV at, for example, a location of mitral regurgitation. In some implementations, the leaflet coaptation assist element 5002 includes a first arm 5008 connected to a second arm 5010 by a joint portion 5012 at a distal end 5013 of the leaflet coaptation assist element 5002. In some implementations, the leaflet coaptation assist element 5002 forms a V-shape in the second deployed position, as shown in FIG. 31. In the first delivery state (not shown), the first arm 5008 and the second arm 5010 are adjacent each other (e.g., parallel or near parallel to each other) to create a smaller profile to be delivered through the delivery system 102. The first arm 5008 includes a first outer surface 5014 and the second arm 5010 includes a second outer surface 5015. The first outer surface 5014 and the second outer surface 5015 are configured to engage the anterior leaflet 20 and the posterior leaflet 22, respectively.

One or more optional retrieval lines 5016 can be attached to the leaflet coaptation assist element 5002 to withdraw the leaflet coaptation assist element 5002. In the illustrated implementation, a retrieval line 5016 is attached to each of the first arm 5008 and the second arm 5010 at a proximal end 5018 of the leaflet coaptation assist element 5002. In other implementations, a single optional retrieval line 5016 can be attached to both of the first and second arms 5008, 5010, or to another portion of the leaflet coaptation assist element 5002, to provide for removal of the element.

In some implementations, the first outer surface 5014 and/or the second outer surface 5015 can be configured to temporarily retain the leaflets 20, 22 against the first arm 5008 and the second arm 5010, respectively, once the leaflets 20, 22 come into contact with the first outer surface 5014 and/or the second outer surface 5015. For example, the first outer surface 5014 and/or the second outer surface 5015 can have a textured or sticky surface (e.g., tissue Velcro, small hooks, etc.) that temporarily resists the leaflets 20, 22 disengaging from the first arm 5008 and the second arm 5010.

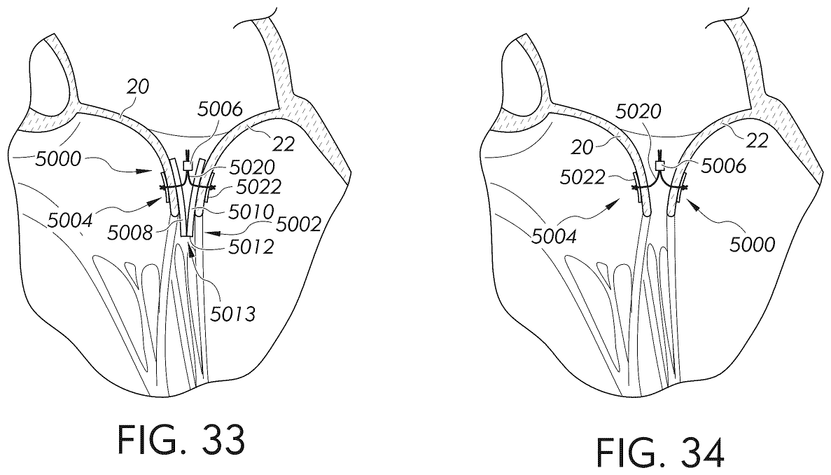

Referring to FIG. 32, once the leaflet coaptation assist element 5002 is in position in the valve annulus, the one or more fixation elements 5004 can be deployed. The one or more fixation elements 5004 can be configured in a variety of ways. Any fixation elements that can engage the leaflets 20, 22 to connect the leaflets together and/or perform an edge-to-edge repair of the leaflets 20, 22 can be used. In the illustrated example of FIG. 33, each of the one or more fixation elements 5004 includes a line 5020 and an anchor 5022. The line 5020 and the anchor 5022 can take a wide variety of different forms. Examples of lines 5020 include, but are not limited to, sutures, wires, cables, chords, bendable rods, any combination thereof, etc. Examples of anchors 5022 include, but are not limited to, pledgets, sufficiently sized knots, stops, or some other line anchoring device.

As shown in FIGS. 32-33, with the leaflet coaptation assist element 5002 in position in the valve, when the leaflets 20, 22 are positioned against, or adjacent, the leaflet coaptation assist element 5002 (e.g., during systole), one anchor 5022 (FIG. 33) is deployed outward (as indicated by the arrow in FIG. 32) through the first leaflet 20 and a second anchor 5022 (FIG. 33) is deployed outward (as indicated by the arrow in FIG. 32) through the second leaflet 22. The anchors 5022 can be deployed through the leaflets simultaneously or one at a time. In some implementations, the first arm 5008 and the second arm 5010 of the leaflet coaptation assist element 5002 can include one or more openings, slots, and/or passages (not shown) extending from an inner surface 5023 of each arm 5008, 5010 to the outer surfaces 5014, 5015. Thus, in some implementations, the anchors 5022 are optionally deployed through the openings (not shown) and then through the leaflets 20, 22.

The anchors 5022 can be deployed through the leaflets 20, 22 by any suitable means. For example, a piercing device (not shown) can be used to create a passage through which the anchors 5022 can pass. Any suitable piercing device (not shown) can be used, such as a needle or a sharp point or a sharp edge on the anchors 5022. In the example of FIG. 33, a single anchor 5022 is deployed in each of the leaflets 20, 22. In other implementations, however, more than a single anchor 5022 can be used in one or both of the leaflets 20, 22. As shown in FIG. 33, each anchor 5022 is attached to a separate line 5020 which extends from the anchor 5022 back through the leaflet 20, 22 and into a delivery catheter 5024 of the delivery system 102.

Referring to FIG. 33, once the anchors 5022 are deployed through the leaflets 20, 22, the anchors 5022 are configured to resist movement back through the openings or passages (not shown) formed in the leaflets 20, 22 (e.g., the anchor forms a shape larger in cross section than the opening in the leaflet). As a result, applying tension to the lines 5020 causes the anchors 5022 to hold the leaflets 20, 22 in a repair position (e.g., in a position where the leaflets are held close together, in a position that inhibits leaflet flail, in contact with the leaflet coaptation assist element, in an edge-to-edge repair condition, in a position the reduces or eliminates valve regurgitation, etc.).

With the lines 5020 tensioned to hold the leaflets 20, 22 in the repair position, the lock 5006 can be deployed to lock the lines 5020 in position, and thus the leaflets 20, 22 in the repair position. The lock 5006 can be configured in a variety of ways. Any suitable locking device capable of securing the lines 5020 in positioned to hold the leaflets 20, 22 in the repair position can be used. For example, the lock 5006 can be a mechanical lock that is advanced over both of the lines 5020 through and out of a distal end 5026 (FIG. 33) of the catheter 5024 to a desired position on the lines 5020 and then locked onto the lines 5020 at that position.

Referring to FIG. 34, once the lock 5006 is deployed and acting to lock the lines 5020, the leaflet coaptation assist element 5002 can optionally be removed. For example, in some implementations, the retrieval lines 5016 can be pulled proximally causing the leaflet coaptation assist element 5002 to move to the elongated condition and be pulled back into the catheter 5024. Thus, the leaflet coaptation assist element 5002 is used during deployment but is removed after the leaflets 20, 22 have been secured. In some implementations, the leaflet coaptation assist element 5002 is left in place with the anchors 5022.

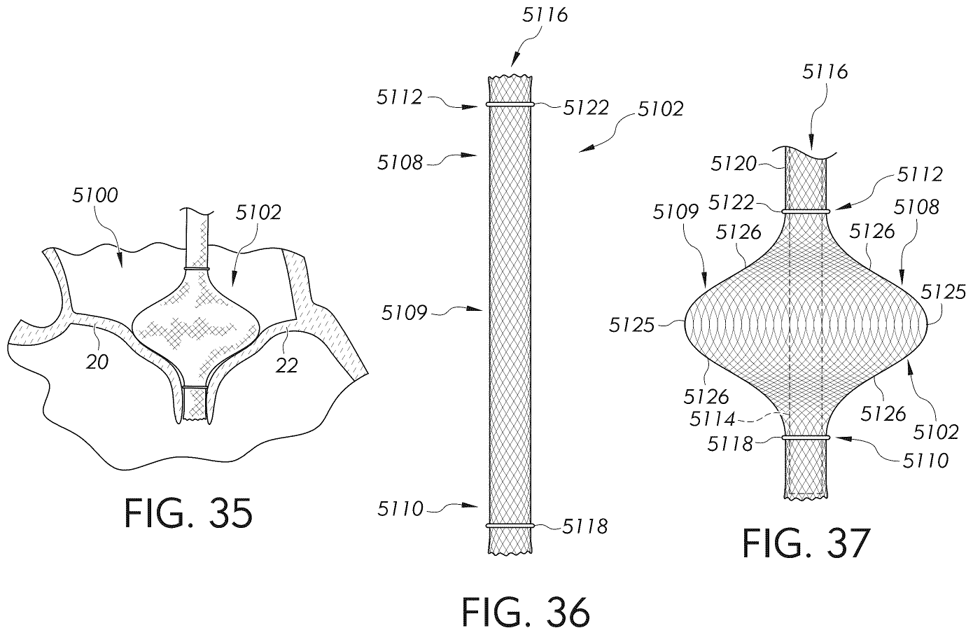

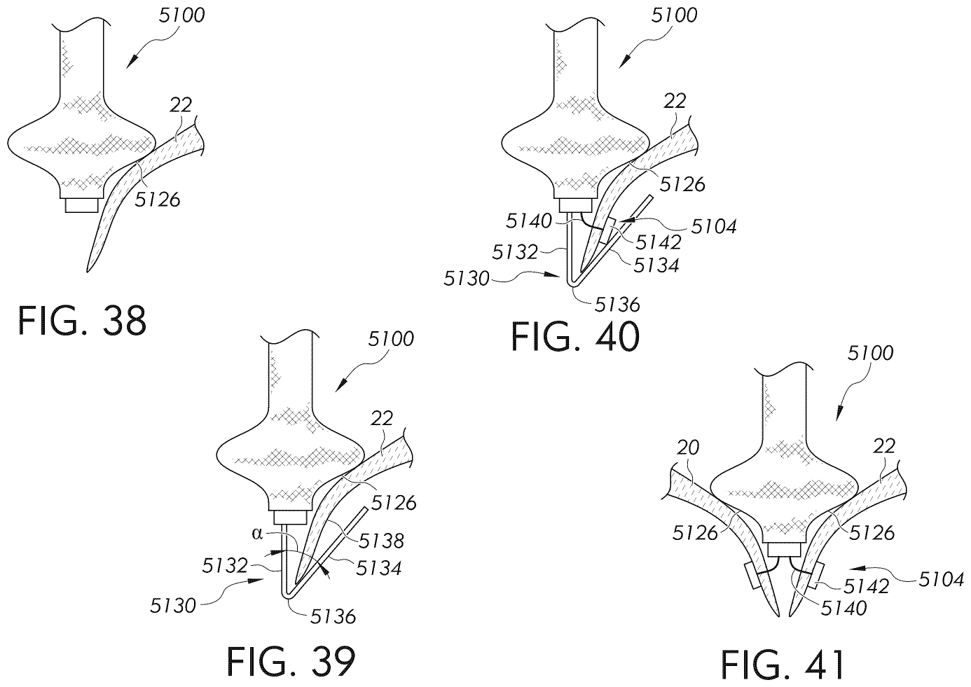

FIGS. 35-44 illustrate a method and a device or implant 5100 (e.g., a prosthetic device, a valve repair device, implantable device, etc.) useable for repairing a native heart valve (e.g., the mitral valve or the tricuspid valve). The device or implant 5100 can include any of the features for another device or implant discussed in the present application or the applications cited herein, and the device 5100 can be positioned to engage valve tissue (e.g., leaflets 20, 22, 30, 32, 34) as part of any suitable valve repair system (e.g., any valve repair system disclosed in the present application, or the applications cited above).

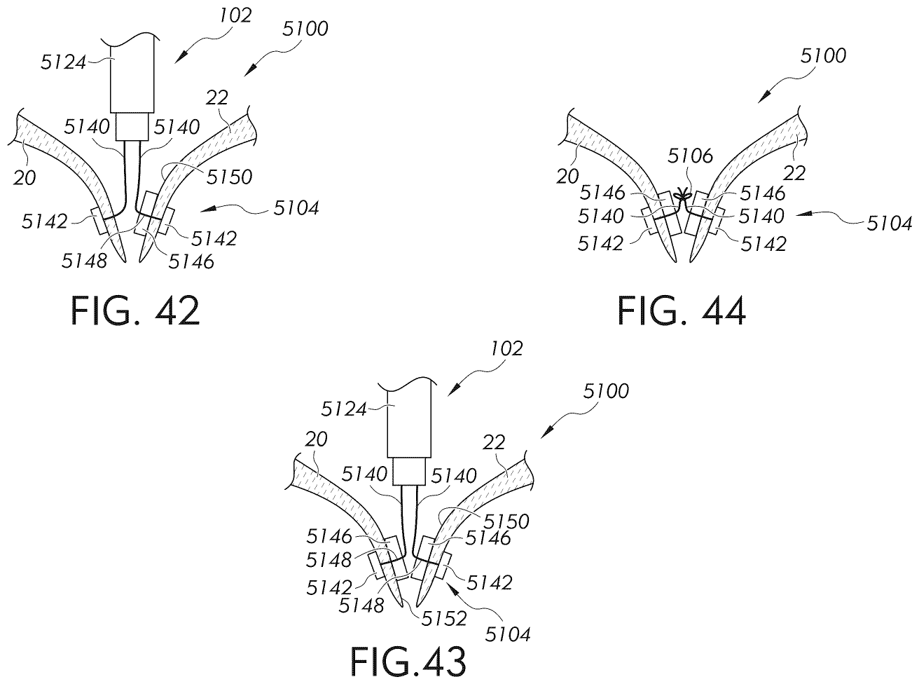

The device or implant 5100 is deployed from a delivery system 102 (FIG. 42). The delivery system 102 can comprise one or more of a catheter, a sheath, a guide catheter/sheath, a delivery catheter/sheath, a steerable catheter, an implant catheter, a tube, a channel, a pathway, combinations of these, etc. The device or implant 5100 includes a leaflet coaptation assist element 5102, one or more fixation elements 5104, and a lock 5106.

The leaflet coaptation assist element 5102 is adapted to inhibit regurgitation and/or suppress and/or prevent the native leaflets (20, 22, 30, 32, 34) from prolapsing or flailing. The coaptation assist element 5102 can be configured in a variety of ways. For example, the size, shape, and materials of the coaptation assist element 5102 can vary. Any configuration that can inhibit regurgitation and/or suppress/or prevent the native leaflets (20, 22, 30, 32, 34) from prolapsing or flailing can be used. Referring to FIG. 35, in some implementations, the coaptation assist element 5102 is configured to be implanted between leaflets 20, 22, 30, 32, 34 of a native valve (e.g., a native mitral valve, native tricuspid valve, etc.) to prevent or inhibit the leaflets from prolapsing. In the example of FIG. 35, the leaflet coaptation assist element 5102 is positioned between the anterior leaflet 20 and the posterior leaflet 22 of the mitral valve MV at, for example, a location of mitral regurgitation. In some implementations, the coaptation assist element 5002 has a first delivery state (e.g., narrow state) suitable for delivery through a catheter and a second deployed state (e.g., expanded or wider state) suitable for blocking the leaflets from prolapsing.

In the example of FIGS. 36-37, the leaflet coaptation assist element 5102 includes a tubular body 5108 adapted to be longitudinally compressed to widen a compressible portion 5109 of the tubular body 5108. The tubular body 5108 can be made of any suitable material or materials. In some implementations, the tubular body 5108 is a braided tube made from any suitable braided materials, such as steel, other metals, plastics, composites, nylon, etc. and/or shape-memory alloys such as Nitinol.

In the example of FIG. 36, in the delivery state, the tubular body 5108 is elongated and suitable for delivery through a lumen of a catheter 5124 of a delivery system 102 (see FIG. 42). The compressible portion 5109 includes a distal end 5110 and a proximal end 5112 opposite the distal end 5110. In the deployed state, as shown in FIG. 37, the compressible portion 5109 of the tubular body 5108 is longitudinally compressed (i.e., the distal end 5110 and the proximal end 5112 are moved axially closer to each other) to widen the compressible portion 5109. The compressible portion 5109 can be configured to be longitudinally compressed in a variety of ways. In some implementations, an actuation element 5114 (e.g., an actuation shaft, actuation tube, actuation wire, actuation rod, etc.) extends through an inner passage 5116 in the compressible portion 5109 and is fixed to the distal end 5110. The actuation element 5114 can be fixed to the distal end 5110 by any suitable manner. In the example of FIGS. 35-37, the distal end 5110 of the compressible portion 5109 is secured to the exterior of the actuation element 5114 by a crimp ring or band 5118.

The proximal end 5112 of the compressible portion 5109 can be configured to be engaged by a positioning element 5120 (e.g., a wire, a rod, a tube, a shaft, a pusher, etc.). In some implementations, the proximal end 5112 includes a stop 5122 which the positioning element 5120 engages. In some implementations, the stop 5122 is a crimp ring or band 5118 and the positioning element 5120 extends within the inner passage 5116, over the actuation element 5114, to abut the stop 5122.

In some implementations, to move the leaflet coaptation assist element 5102 from the delivery state to the deployed state, the positioning element 5120 is pushed against the stop 5122 while the actuation element 5114 is held in position. Thus, the proximal end 5112 is moved distally along the actuation element 5114. As a result, the proximal end 5112 moves closer to the distal end 5110 causing the tubular body 5108 of the compressible portion 5109 to widen or expand outward. For example, in the example of FIG. 37, the tubular body 5108 of the compressible portion 5109 widens to form a “spinning top” shape. A spinning top shape can be characterized, generally, as a rhombus having a rounded corner 5125 between lateral side surfaces 5126.

In some implementations, instead of pushing the proximal end 5112 distally along the actuation element 5114 to widen the compressible portion 5109, the positioning element 5120 can be held in position against the stop 5122 and the actuation element 5114 can be pulled proximally. Thus, since the actuation element 5114 is fixed to the distal end 5110, the distal end 5110 moves proximally with the actuation element 5114. Since the proximal end 5112 is held in place by the positioning element 5120, the distal end 5110 moves closer to the proximal end 5112 causing the tubular body 5108 of the compressible portion 5109 to widen or expand outward. The width of the compressible portion 5109 in the deployed state can be adjusted based on the native anatomy by selecting how far the proximal end 5112 is moved relative to the distal end 5110, or vice versa.

The lateral side surfaces 5126, in particular the distally facing lateral side surfaces, are configured to engage the leaflets 20, 22 during systole. As shown in FIGS. 35 and 41, when the leaflet coaptation assist element 5102 is positioned within the valve annulus and is in the deployed state, the leaflets 20, 22 come into contact with distally facing lateral side surfaces such that the distally facing lateral side surfaces block the leaflets 20, 22 from prolapsing. In some implementations, the method and device 5100 can be configured for temporarily retaining the leaflets 20, 22 against, or adjacent, the distally facing lateral side surfaces 5126 once the leaflets 20, 22 come into contact with the distally facing lateral side surfaces 5126. The device 5100 can be configured for retaining the leaflets 20, 22 in a variety of ways. For example, the distally facing lateral side surfaces 5126 can have a textured surface or sticky surface (e.g., tissue Velcro, small hooks, etc.) that temporarily resists the leaflets 20, 22 disengaging from distally facing lateral side surfaces 5126.

Alternatively, or in conjunction with a textured or sticky surface, the device can be configured for retaining the leaflets 20, 22 by including a retaining member 5130 configured to engage the leaflet 20, 22 to retain the leaflet 20, 22 against, or adjacent, the distally facing lateral side surface 5126. The retaining member 5130 can be configured in a variety of ways. In the example of FIGS. 39-40, the retaining member 5130 includes a stem 5132 extending distally from the leaflet coaptation assist element 5102 and an arm 5134 extending proximally from a distal end 5136 of the stem 5132 at an angle, such as an acute angle. The arm 5134 is configured to extend around to a ventricular side 5138 of the leaflet 20, 22 to trap the leaflet between the arm 5134 and the stem 5132 and/or the leaflet coaptation assist element 5102.

Referring to FIGS. 39-40, the retaining member 5130 can be deployed through the inner passage 5116 (FIGS. 36-37) of the leaflet coaptation assist element 5102. In a delivery state, the stem 5132 and the arm 5134 can be adjacent each other (e.g., parallel or near parallel to each other) or can be in-line with each other (e.g., the arm extends from the stem in a straight line) to create a smaller profile for delivery through the delivery system 102 and inner passage 5116. In a deployed state, the arm 5134 bends or flexes away from the stem 5132 at an angle α (e.g., 30-50 degrees) sufficient to capture the leaflet 22 between the stem 5132 and the arm 5134.

Referring to FIG. 40, once the leaflet coaptation assist element 5102 is in position in the valve annulus and the optional retaining member 5130 has optionally captured the leaflet 22, the one or more fixation elements 5104 can be deployed. The one or more fixation elements 5104 can be configured in a variety of ways. Any fixation elements that can engage the leaflets 20, 22 to perform an edge-to-edge repair of the leaflets 20, 22 can be used. In the illustrated example of FIG. 40, each of the one or more fixation elements 5104 includes a line 5140 and an anchor 5142. The line 5140 and the anchor 5142 can take a wide variety of different forms. Examples of lines 5140 include, but are not limited to, sutures, wires, cables, chords, bendable rods, any combination thereof, etc. Examples of anchors 5142 include, but are not limited to, pledgets, sufficiently sized knots, stops, or some other line anchoring device.

Referring to FIG. 40, as discussed regarding the anchors 5022 of FIG. 33, one of the anchors 5142 is deployed outward through the posterior leaflet 22. The anchor 5142 can be deployed through the leaflet 22 by any suitable means. For example, a piercing device (not shown) can be used to create a passage through which the anchor 5142 can pass. Any suitable piercing device (not shown) can be used, such as a needle or a sharp point or a sharp edge on the anchors 5142. The anchor 5142 is attached to the line 5140 which extends from the anchor 5142 back through the leaflet 22, through the inner passage 5116 of the leaflet coaptation assist element 5102, and into the delivery catheter 5124 of the delivery system 102 (FIG. 42).

Once the anchor 5142 is deployed through the leaflet 22, the anchor 5142 is configured to resist movement back through the opening or passage (not shown) formed in the leaflet 22 (e.g., form a shape larger in cross section than the opening in the leaflet). In the same manner as described regarding the anchor 5142 through the leaflet 22, another anchor 5142 can be deployed through the anterior leaflet 22. In the example of FIG. 41, a single anchor 5142 is deployed in each of the leaflets 20, 22. In other implementations, however, more than a single anchor 5142 can be used in one or both of the leaflets 20, 22. The anchors 5142 can be deployed through the leaflets 20, 22 simultaneously or one at a time.

Referring to FIG. 41-42, once the anchors 5142 are deployed through the leaflets 20, 22, applying tension to the lines 5140 causes the anchors 5142 to hold the leaflets 20, 22 in a repair position (e.g., in a position where the leaflets are held close together, in a position that inhibits leaflet flail, in contact with the leaflet coaptation assist element, in an edge-to-edge repair condition, etc.). The leaflet coaptation assist element 5102 can then optionally be removed. Thus, the leaflet coaptation assist element 5102 is used during deployment but is removed after the leaflets 20, 22 have been secured in some implementations. For example, the leaflet coaptation assist element 5102 can be returned to the delivery position by moving the proximal end 5112 away from the distal end 5110, or vice versa, in a similar manner as described above regarding moving the leaflet coaptation assist element 5102 to the deployed position. In some implementations, the leaflet coaptation assist element 5102 can be left in place and secured to the anchors.

In some implementations, the compressible portion 5109 of the tubular body 5108 can be biased to the elongated delivery state. For example, in some implementations, the compressible portion 5109 can use spring materials, such as steel, other metals, plastics, composites, etc. that bias the compressible portion 5109 to the elongated delivery state, or the compressible portion 5109 can use shape-memory alloys, such as Nitinol, which are shape-set to the elongated delivery state.

In some implementations, the compressible portion 5109 of the tubular body 5108 can be biased to the expanded deployed state. For example, in some implementations, the compressible portion 5109 can use spring materials, such as steel, other metals, plastics, composites, etc. that bias the compressible portion 5109 to the expanded deployed state, or the compressible portion 5109 can use shape-memory alloys, such as Nitinol, which are shape-set to the expanded deployed state.

In some implementations, the device 5100 can include one or more optional spacers 5146 (FIGS. 42-43). The one or more spacers 5146 are configured to fill all of, or a portion of, the gap between the leaflets 20, 22 after the leaflets are secured by the anchors 5142. The spacers 5146 can also prevent or inhibit the line 5140 from cutting into the tissue of the leaflets 20, 22 surrounding the passages in the leaflets 20, 22 through which the lines 5140 extend. The spacers 5146 can be configured in a variety of ways, including different shapes and sizes. Any suitable shape and size may be used. Furthermore, any suitable number of spacers 5146 may be used. In some implementations, the spacers 5146 are configured to be deployed by sliding along the lines 5140.

In the illustrated example of FIGS. 42-43, the spacers 5146 can be generally disk-shaped with a central passage 5148 configured to receive the line 5140. In other examples, however, the spacers 5146 can be configured other than generally disk-shaped with a central passage. A first spacer 5146 can be advanced down the line 5140 associated with the anchor 5142 on the posterior leaflet 22 to an inner surface 5150 of the posterior leaflet 22, as shown in FIG. 42. A second spacer 5146 can be advanced down the line 5140 associated with the anchor 5142 on the anterior leaflet 22 to an inner surface 5152 of the anterior leaflet 20, as shown in FIG. 43. While the example of FIGS. 42-43 illustrates a pair of spacers 5146, a single spacer may be used or additional spacers may be added as needed to fill the gap, or a portion thereof.

Referring to FIG. 44, with the spacers 5146 in place and the lines 5140 tensioned to hold leaflet 20, 22 in the repair position, the lock 5106 can be used to lock the lines 5140, and thus the leaflets 20, 22 in the repair position. The lock 5106 can be configured in a variety of ways. Any suitable locking device capable of securing the lines 5140 in positioned to hold the leaflets 20, 22 in the repair position can be used. For example, in the illustrated implementation of FIG. 44, the lock 5106 is one or more knots formed in the lines 5140. In some implementations, the lock 5106 can be a mechanical lock that is advanced over both of the lines 5140 through, and out of a distal end, of the catheter 5124 to a desired position on the lines 5140 and then locked onto the lines at that position. In some implementations, the lock is a fastener, clamp, clip, etc. that connects the two lines together.

The leaflet coaptation assist element 5102 can optionally be removed. For example, in some implementations, the leaflet coaptation assist element 5102 can be returned to the elongated configuration (see FIG. 36) and can be pulled proximally back into the catheter. This can be done at any point after the anchor(s) 5142 are deployed. Thus, the leaflet coaptation assist element 5102 is used during deployment but is removed after the leaflets 20, 22 have been secured. In some implementations, the leaflet coaptation assist element 5002 is left in place with the anchors 5142.

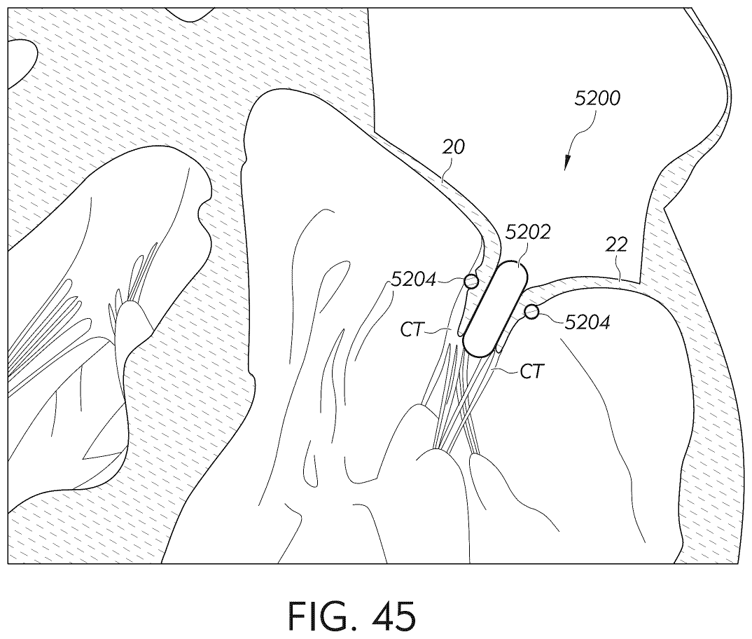

FIGS. 45-50 schematically illustrate a method and a device or implant 5200 (e.g., a prosthetic device, a valve repair device, implantable device, etc.) useable for repairing a native heart valve (e.g., the mitral valve or the tricuspid valve). The device or implant 5200 can include any of the features for another device or implant discussed in the present application or the applications cited above, and the device 5200 can be positioned to engage valve tissue (e.g., leaflets 20, 22, 30, 32, 34) as part of any suitable valve repair system (e.g., any valve repair system disclosed in the present application, or the applications cited above).

The device or implant 5200 is deployed from a delivery system (e.g., delivery system 102). The delivery system can comprise one or more of a catheter, a sheath, a guide catheter/sheath, a delivery catheter/sheath, a steerable catheter, an implant catheter, a tube, a channel, a pathway, combinations of these, etc. The device or implant 5200 can include an optional coaptation element 5202 (e.g., spacer, coaption element, gap filler, membrane, sheet, plug, wedge, balloon, etc.) and one or more fixation elements 5204.

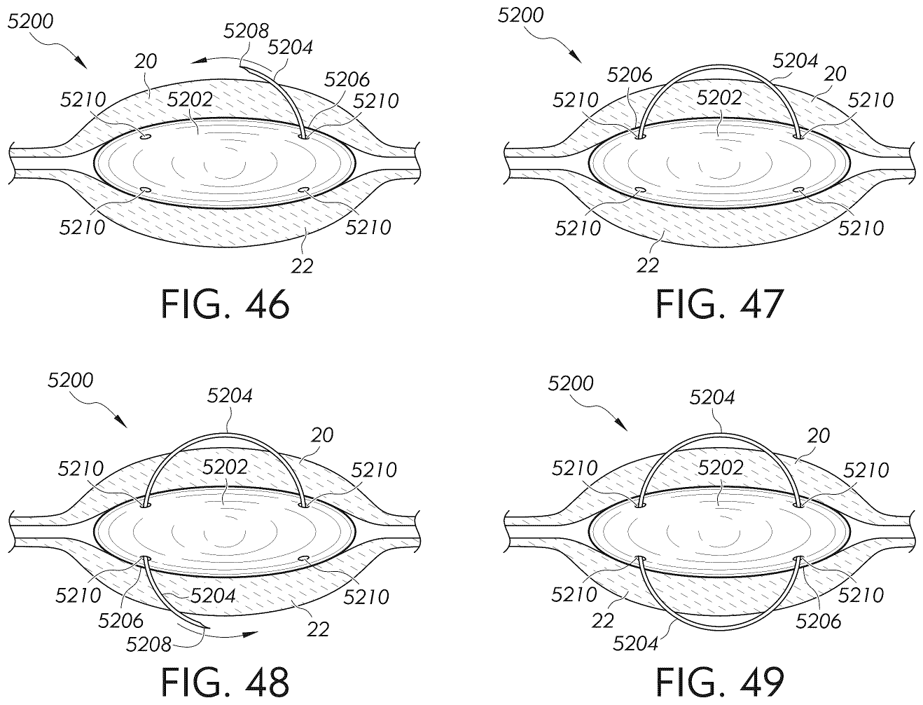

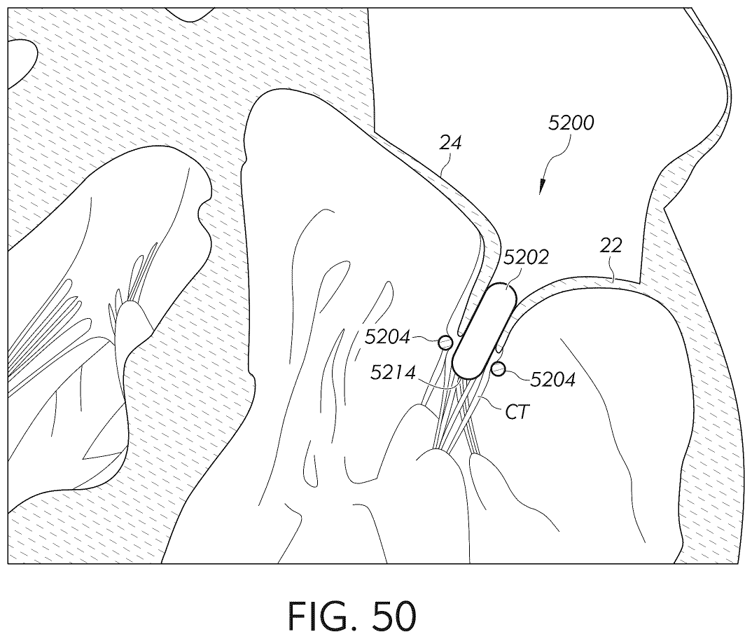

The coaptation element 5202 is configured to be positioned within the native heart valve orifice to help fill the space between the leaflets and form a more effective seal, thereby reducing or preventing or inhibiting regurgitation described above. The coaptation element 5202 can be configured in a variety of ways. For example, in some implementations, the coaptation element 5202 can have a structure that is impervious to blood (or that resists blood flow therethrough) and that allows the native leaflets to close around the coaptation element 5202 during ventricular systole to block blood from flowing from the left or right ventricle back into the left or right atrium, respectively. The coaptation element 5202 can have various shapes. In some implementations, the coaptation element 5202 can have an elongated cylindrical shape having a round cross-sectional shape, an oval cross-sectional shape, an ovoid cross-sectional shape, a crescent cross-sectional shape, a rectangular cross-sectional shape, or various other non-cylindrical shapes. In the example of FIGS. 45-50, the coaptation element 5202 has an oval cross-sectional shape when viewed from the front (as shown in FIGS. 45 and 50) and when viewed from the top (as shown in FIGS. 46-49).

The fixation elements 5204 (which can also be referred to as capture elements) are configured to capture the valve leaflets 20, 22, 30, 32, 34 and/or the chordae tendineae CT against the coaptation element 5202 to restore leaflet coaptation of the valve. The fixation elements/capture elements 5204 can be configured in a variety of ways (e.g., wire, line, suture, braid, clip, rod, tube, arm, hook, etc.). In the example of FIGS. 45-50, the device 5200 includes a pair of fixation elements 5204 connected to the coaptation element 5202. In some implementations, however, the device 5200 can include more than a pair of fixation elements 5204 or only a single fixation element.

In the example of FIGS. 45-50, the capture elements or fixation elements 5204 are formed as a wire or hook configured to move in an arc around and/or through the valve tissue (i.e., valve leaflets 20, 22 and/or the tendineae CT). In some implementations, the wire includes a shape-setting material, (e.g., a nitinol) that is shape-set to extend from the coaptation element 5202 in an arc and curve back toward the coaptation element 5202 to capture and/or pierce the valve leaflets 20, 22 and/or the chordae tendineae CT. As such, the capture elements or fixation element(s) 5204 hold the leaflets 20, 22 against the coaptation element 5202. In some implementations, each of the fixation elements 5204 form a closed loop with the coaptation element 5202.

The capture elements or fixation elements 5204 can be deployed in a variety of ways. In the example of FIGS. 46-49, prior to the deployment of each fixation element 5204, the fixation element 5204 is positioned within the coaptation element 5202. To deploy, one of the fixation elements 5204 can be advanced (e.g., pushed) out of an exterior surface 5206 (e.g., a side surface) of the coaptation element 5202, as shown in FIG. 46. In some implementations, such as when leaflet tissue is to be captured, the fixation element 5204 can have a sharp or pointed, distal tip 5208 configured to pierce through the leaflet tissue.

As shown in FIGS. 46-47, as the capture element or fixation element 5204 is advanced from the coaptation element 5202, its tip 5208 moves in an arc through and/or around the tissue of the anterior leaflet 20 (and/or optionally tissue of the chordae tendineae CT) and returns to coaptation element 5202 at a location along the exterior surface 5206 (e.g., side surface) spaced apart from where the fixation element 5204 advanced out of. In some implementations, the tip 5208 of the fixation element 5204 reenters the coaptation element 5202 through the exterior surface 5206 to form a closed shape (e.g., a loop). In the illustrated example of FIGS. 46-49, the coaptation element 5202 can include pre-formed apertures 5210 at the locations where the fixation element 5204 exits and reenters the coaptation element 5202. Alternatively, in some implementations, the tip 5208 of the fixation element 5204 pierces the exterior surface 5206 to reenter the coaptation element 5202. Further, in some implementations, the tip 5208 of the fixation element 5204 stops advancing prior to reentering the coaptation element 5202 (e.g., abuts or is adjacent the exterior surface 5206).

Referring to FIGS. 48-49, another capture element or fixation element 5204 can be advanced from the coaptation element 5202 to pierce and/or capture tissue of the posterior leaflet 22 (and/or optionally tissue of the chordae tendineae CT). In the same manner as described above regarding capturing the tissue of the anterior leaflet 20, as the fixation element 5204 is advanced from the coaptation element 5202, its tip 5208 moves in an arc through and/or around the tissue of the posterior leaflet 20 (and optionally tissue of the chordae tendineae CT) and returns to coaptation element 5202 (e.g., pierces, extends through, abuts, or is adjacent the exterior surface 5206) at a location spaced apart from where the fixation element 5204 advanced out of. In some implementations, the fixation element 5204 that captures the anterior leaflet 20 is positioned on an opposite side of the coaptation element 5202 as the fixation element 5204 that captures the posterior leaflet 22.

FIG. 50 illustrates the device 5200 of FIGS. 45-49 positioned within the native heart valve orifice between the leaflets 20, 22. In some implementations, the device 5200 is positioned within the native heart valve orifice and/or configured to capture the chordae tendineae CT without capturing the tissue of the valve leaflets 20, 22. In some implementations, as shown in FIG. 50 (as compared to FIG. 45), the coaptation element 5202 is configured such that the capture elements or fixation elements 5204 are deployed from the coaptation element 5202 at a location closer to a bottom end 5214 (i.e., ventricular end) of the coaptation element 5202. Thus, the coaptation element 5202 can still be positioned optimally between the leaflets such that the valve leaflets 20, 22 form a more effective seal, but the capture elements/fixation elements 5204 are deployed below the valve leaflets 20, 22 to capture the chordae tendineae CT without capturing the leaflets 20, 22. In some implementations, instead of, or in conjunction with, the device 5200 being configured to deploy the fixation elements 5204 at a location closer to a bottom end 5214 and below the valve leaflets 20, 22, the coaptation element 5202 can be positioned lower in the valve orifice (i.e., closer to the ventricle). Thus, when positioned lower in the valve orifice, the capture elements/fixation elements 5204, when deployed, are below the valve leaflets 20, 22 to capture the chordae tendineae CT without piercing and/or capturing the leaflets 20, 22.

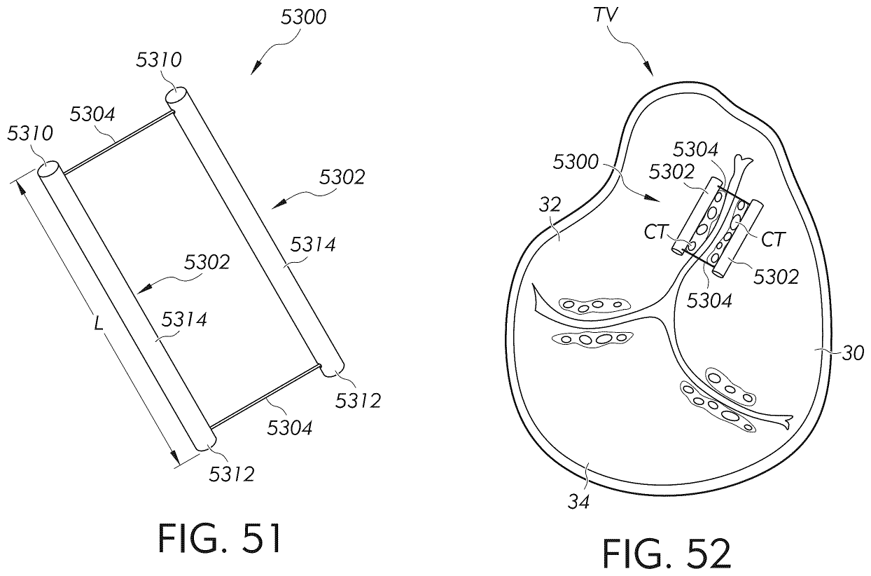

FIGS. 51-52 schematically illustrate a method and a device or implant 5300 (e.g., a prosthetic device, a valve repair device, implantable device, etc.) useable for repairing a native heart valve (e.g., the mitral valve or the tricuspid valve). The device or implant 5300 can include any of the features for another device or implant discussed in the present application or the applications cited above, and the device 5300 can be positioned to engage valve tissue (e.g., leaflets 20, 22, 30, 32, 34) as part of any suitable valve repair system (e.g., any valve repair system disclosed in the present application, or the applications cited above).

The device or implant 5300 is deployed from a delivery system (e.g., delivery system 102). The delivery system can comprise one or more of a catheter, a sheath, a guide catheter/sheath, a delivery catheter/sheath, a steerable catheter, an implant catheter, a tube, a channel, a pathway, combinations of these, etc. In some implementations, the device or implant 5300 is configured to restore leaflet coaptation via chordae approximation and includes one or more capture elements 5302 and one or more attachment elements 5304.

In some implementations, the capture elements 5302 are adapted to engage tissue, such as chordae tendineae CT (e.g., the primary chordae (closest to the edge of the leaflet)) of two or more leaflets. In some implementations, the capture elements 5302 are configured as chordae capture elements adapted to engage chordae tendineae. However, the capture elements 5302 can be adapted to engage a variety of tissues, such as leaflets, annulus tissue, chordae, etc., and while many examples herein involve capturing chordae tendineae, the principles and features can be similarly used to capture other types of tissue.

The one or more attachment elements 5304 are configured to connect the capture elements 5302 to each other and pull, under tension, the capture elements 5302 together; thereby pulling the leaflets together to reduce the gap between the leaflets. Each capture element 5302 can be configured in a variety of ways, such as for example, as a wire, a rod, a shaft, a tube, a line, a strip, a hook, etc.

In some implementations, the capture element 5302 can be configured to engage multiple chordae tendineae CT to distribute the tension force between multiple chordae to protect the chordae from being overloaded.

In the example of FIGS. 51-52, the device 5300 includes a pair of capture elements 5302. In other implementations, the device 5300 can utilize more than a pair of capture elements 5302. Each of the capture elements 5302 can be configured as a cylindrical rod having a first end 5310, a second end 5312 opposite the first end 5310, and a cylindrical outer side surface 5314. The capture element 5302 can have a length L configured to allow the capture element 5302 to engage multiple chordae tendineae CT.

The capture elements 5302 are configured to be attached to each other by one or more of the attachment elements 5304. The attachment elements 5304 can be configured in a variety of ways, such as for example, as lines, sutures, wires, cables, chords, braids, etc. In the example of FIGS. 51-52, the attachment elements 5304 are configured as suture lines attached to and extending between the capture elements 5302. In the illustrated example, one attachment element 5304 extends between the capture elements 5302 proximate the first end 5310 and a second attachment element 5304 extends between the capture elements 5302 proximate the second end 5312. The attachment elements 5304 can attach to the capture elements 5302 in any suitable manner.

Referring to FIG. 52, the tricuspid valve TV is illustrated as being repaired. The device 5300, however, can be used to repair other valves, such as the mitral valve MV.

In some implementations, to deploy the device 5300, the capture elements 5302 are placed behind the chordae (e.g., the primary chordae), one for each leaflet 30, 32. In some implementations, the attachment elements 5302 are routed between the positioned capture elements 5302 at, or near, the first end 5310 of each capture element 5302 and at, or near, the second end 5312 of each capture element 5302.

In some implementations, the attachment elements 5304 can be cinched together in order to pull the capture elements 5302. As a result, the leaflets 30, 32, are pulled toward each other and/or into a straight or substantially straight line, reducing the gap between the leaflets 30, 32.

In some implementations, the cinched attachment elements 5304 can be locked in the place to hold the leaflets 30, 32 in the repair position by any suitable lock (not shown) such as a mechanical lock, one or more knots in a suture line, etc.

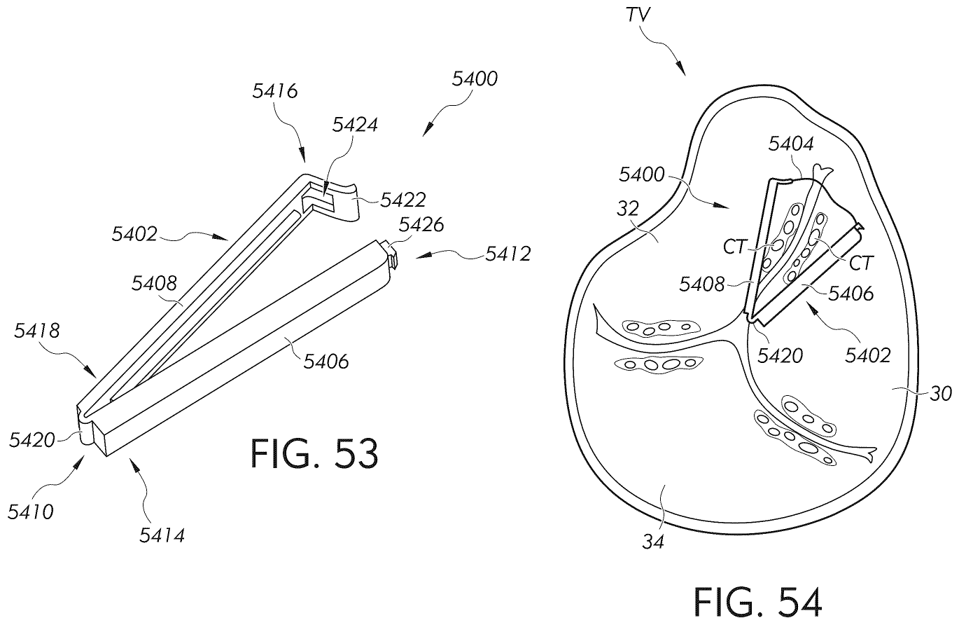

FIGS. 53-54 schematically illustrate a method and a device or implant 5400 (e.g., a prosthetic device, a valve repair device, implantable device, etc.) useable for repairing a native heart valve (e.g., the mitral valve or the tricuspid valve). The device or implant 5400 can include any of the features for another device or implant discussed in the present application or the applications cited above, and the device 5400 can be positioned to engage valve tissue (e.g., leaflets 20, 22, 30, 32, 34) as part of any suitable valve repair system (e.g., any valve repair system disclosed in the present application, or the applications cited above).

The device or implant 5400 is deployed from a delivery system (e.g., delivery system 102). The delivery system can comprise one or more of a catheter, a sheath, a guide catheter/sheath, a delivery catheter/sheath, a steerable catheter, an implant catheter, a tube, a channel, a pathway, combinations of these, etc. In some implementations, the device or implant 5400 is configured to restore leaflet coaptation, e.g., via chordae approximation, leaflet approximation, tissue approximation, etc.).

In some implementations, the device or implant 5400 includes one or more capture elements 5402 and one or more optional attachment elements 5404. The capture elements 5402 are adapted to engage tissue.

In some implementations, the capture elements 5402 are configured as chordae capture elements adapted to engage chordae tendineae CT (e.g., the primary chordae (closest to the edge of the leaflet)) of two or more leaflets to pull the leaflets together; thus, reducing the gap between the leaflets. However, the capture elements 5402 can be adapted to engage a variety of tissue, such as leaflets, annulus tissue, etc., and while examples may be focused on chordae tendineae, the principles and features can be used with other tissue.

In some implementations, the one or more optional attachment elements 5404 are configured to pull, under tension, the capture elements 5302 together and/or retain the capture elements 5402 in a captured state. Each capture element 5402 can be configured in a variety of ways, such as for example, a rod, a shaft, a tube, a strip, etc. In some implementations, the capture element 5402 can be configured to engage multiple chordae tendineae CT to distribute the tension force between multiple chordae to protect the chordae from being overloaded.

In the example of FIGS. 53-54, the device 5400 includes a pair of capture elements 5402 connected to each other by a joint portion 5410 to form a clip or clasp. The pair of capture elements 5402 can be configured in a variety of ways, including different cross-sectional shapes, different sizes (e.g., length, width, thickness), composed of different materials, etc. In the example of FIGS. 53-54, each of the capture elements 5402 is generally straight with a rectangular cross-section. The first capture element 5406 has a first distal end 5412 and a first proximal end 5414 opposite the first distal end 5412. The second capture element 5408 has a second distal end 5416 and a second proximal end 5418 opposite the second distal end 5416. The joint portion 5410 connects the first proximal end 5414 to the second proximal end 5418 via a hinge 5420 (e.g., a living hinge) such that the first capture element 5406 and the second capture element 5408 can pivot toward and away from each other between an open position (as shown in FIG. 53) and a repair position.

In some implementations, the first distal end 5412 and the second distal end 5416 can be configured to be attached together. The first distal end 5412 and the second distal end 5416 can be attached together in a variety of ways, such as a clasp, a suture line, a wire, a catch, a latch, a detent, or other attachment mechanism. In the example of FIGS. 53-54, the first distal end 5412 includes a latch 5422 projecting inward toward the second distal end 5416. The latch 5422 includes an aperture 5424 configured to receive a projection 5426 on the second distal end 5416. When the projection 5426 is received in the aperture 5424, the first capture element 5406 and the second capture element 5408 are held together in a captured state.

The capture elements 5402 can be made from any suitable materials, such as, for example, spring materials, such as steel, other metals, plastics, composites, etc. or shape-memory alloys such as Nitinol.

Referring to FIG. 54, the tricuspid valve TV is illustrated as being repaired. The device 5400, however, can be used to repair other valves, such as the mitral valve MV.

In some implementations, the device 5400 can be deployed in the open state such that the first capture element 5406 is placed behind the chordae (e.g., the primary chordae) of one leaflet (e.g., the anterior leaflet 30) and the second capture element 5408 is placed behind the chordae (e.g., the primary chordae) of another leaflet (e.g., septal leaflet 32).

In some implementations, to capture the chordae and/or other tissue, the first and second capture elements 5406, 5408 are flexed or pivoted about the hinge 5420 toward each other. The first and second capture elements 5406, 5408 can be pivoted toward each other in a variety of ways. In some implementations, a line (e.g., suture) is routed to both of the positioned first and second capture elements 5406, 5408 and cinched to pull the ends of the first and second capture element 5406, 5408 together.

In some implementations, once the first and second capture elements 5406, 5408 are drawn together, the latch 5422 can be secured onto the projection 5426 to hold the first and second capture element 5406, 5408, and thereby the leaflets 30, 32, in the repair position.

In some implementations, instead of, or in conjunction with the latch 5422 and projection 5426 holding the capture element 5406, 5408 in the repair position, the attachment member 5404 or one or more supplemental suture lines can be used to secure the capture element 5406, 5408 in place. For example, a lock (not shown), such as a mechanical lock or one or more knots in a suture line, can secure the capture elements 5406, 5408 in the repair position. In some implementations, the capture elements 5406, 5408 can be biased toward the repair position. For example, the device 5400 can include spring materials that bias the capture elements 5406, 5408 toward each other or include a shape-memory alloy (e.g., nitinol) that is shape set such that the capture elements 5406, 5408 are biased inward toward each other.

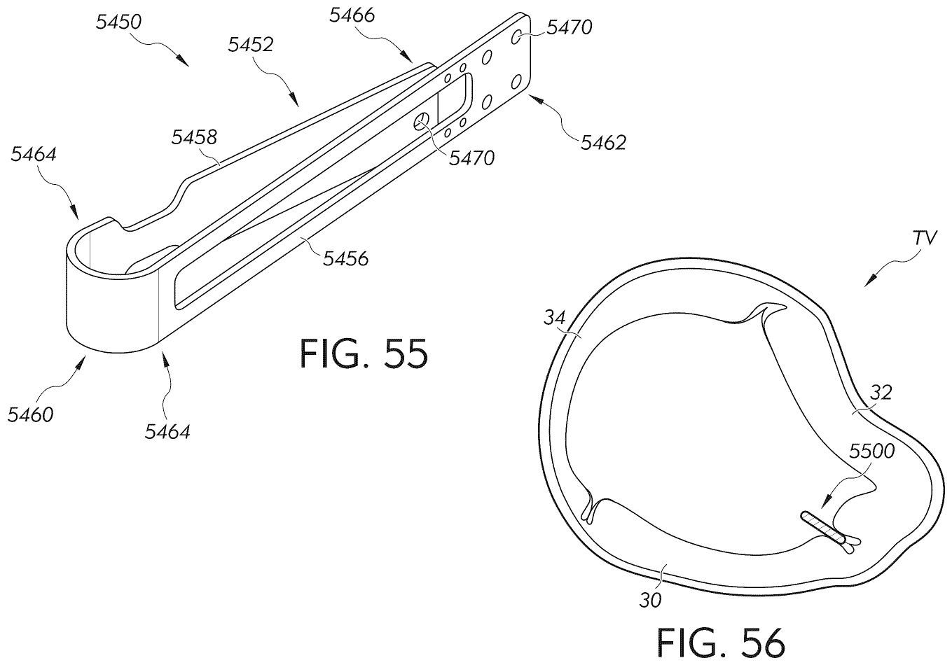

FIG. 55 illustrates a device or implant 5450 (e.g., a prosthetic device, a valve repair device, implantable device, etc.) useable for repairing a native heart valve (e.g., the mitral valve, the tricuspid valve, or another valve). The device 5450 is similar to the device 5400 described regarding FIGS. 53-54, and the description of the device 5400 applies equally to the device 5450.

In some implementations, the device 5450 includes fixation elements or capture elements 5452 configured as a clip having a first capture element 5456 connected to a second capture element 5458 by a joint portion 5460. The first capture element 5456 and the second capture element 5458 can be configured in a variety of ways, including different cross-sectional shapes, different sizes (e.g., length, width, thickness), composed of different materials, etc.

In some implementations, as illustrated by the example of FIG. 55, the first capture element 5456 and the second capture element 5458 are configured as elongated, flat, thin, rectangular strips or panels. The first capture element 5456 has a first distal end 5462 and a first proximal end 5464 opposite the first distal end 5462. The second capture element 5458 has a second distal end 5466 and a second proximal end 5468 opposite the second distal end 5466. The joint portion 5460 connects the first proximal end 5464 to the second proximal end 5468. The joint portion 5460 is configured to allow the first capture element 5456 and the second capture element 5458 to pivot or flex toward and away from each other between an open position and a repair position (FIG. 55).

The capture elements 5452 can be deployed in the same manner as described regarding the capture elements 5402 (e.g., positioned behind the chordae of two leaflets and then flex or pivot about the joint portion inward toward each other).

In some implementations, a suture line or other attachment member (not shown) can be coupled to the distal ends 5462, 5466 to move the capture elements 5456, 5458 and/or secure the capture elements 5456, 5458 in the repair position. For example, in some implementations, the capture elements 5456, 5458 include apertures 5470 proximal the distal ends 5462, 5466 for receiving a suture line or other attachment member (not shown) to provide for movement of the capture elements 5456, 5458 and/or securing the capture elements in the repair position.

In some implementations, the capture elements 5456, 5458 can be biased toward the repair position. For example, the capture elements 5452 can include spring materials that bias the capture elements 5456, 5458 toward each other or include a shape-memory alloy (e.g., nitinol) that is shape set such that the capture elements 5456, 5458 are biased inward toward each other.

FIGS. 56-58 schematically illustrate a method and a device or implant 5500 (e.g., a prosthetic device, a valve repair device, implantable device, etc.) useable for repairing a native heart valve (e.g., the mitral valve or the tricuspid valve). The device or implant 5500 can include any of the features for another device or implant discussed in the present application or the applications cited herein, and the device 5500 can be positioned to engage valve tissue (e.g., leaflets 20, 22, 30, 32, 34) as part of any suitable valve repair system (e.g., any valve repair system disclosed in the present application, or the applications cited above). The device or implant 5500 can be used by itself to repair a native heart valve or can be used in conjunction with another repair device, such as for example, any valve repair device disclosed herein.

The device or implant 5500 is deployed from a delivery system (e.g., delivery system 102). The delivery system can comprise one or more of a catheter, a sheath, a guide catheter/sheath, a delivery catheter/sheath, a steerable catheter, an implant catheter, a tube, a channel, a pathway, combinations of these, etc.

The device or implant 5500 is configured to grasp or attach to leaflet tissue when leaflet tissue comes into contact with the device 5500. When placed between two leaflets, both leaflets can attach to the device 5500 and be held against the device. In this manner, the device 5500 can hold the leaflets together to restore coaptation. The device 5500 can be configured to attach to leaflet tissue in a variety of ways, such as for example, with barbs, hooks, or other structure for attaching to leaflet tissue. In some implementations, the device 5500 passively attaches to the leaflet tissue (i.e., the native leaflets can be allowed to passively contact and attach to the device upon contact, without needing to actively actuate any clamping mechanisms or other attachment devices).

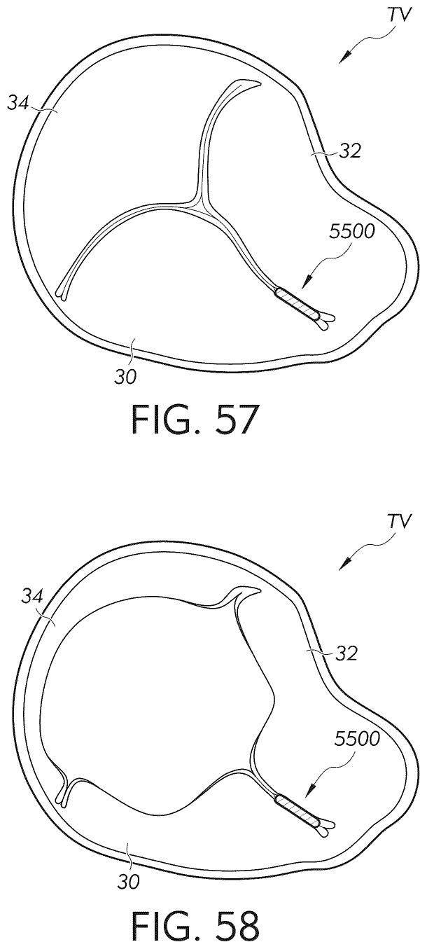

In the example of FIGS. 56-58, the device 5500 is shown positioned at the commissure of the anterior leaflet 30 and the septal leaflet 32 of the tricuspid valve TV. FIG. 56 illustrates the tricuspid valve TV in the diastolic phase, or diastole (i.e., leaflets 30, 32, 34 are open). During the systolic phase, or systole, the leaflets 30, 32, 34 close. When the leaflets 30, 32, 34 close, the tissue of the anterior leaflet 30 and the septal leaflet 32 adjacent the device 5500 attach to the device 5500, as shown in FIG. 57. During the subsequent diastolic phase, the leaflets 30, 32, 34 open, but the portion of the anterior leaflet 30 and the septal leaflet 32 that are attached to the device 5500 are held in the closed condition by the device 5500, as shown in FIG. 58. In this manner, the device can reduce the effective size of a regurgitant valve and/or close a regurgitant area of the valve to reduce regurgitant flow through the valve.

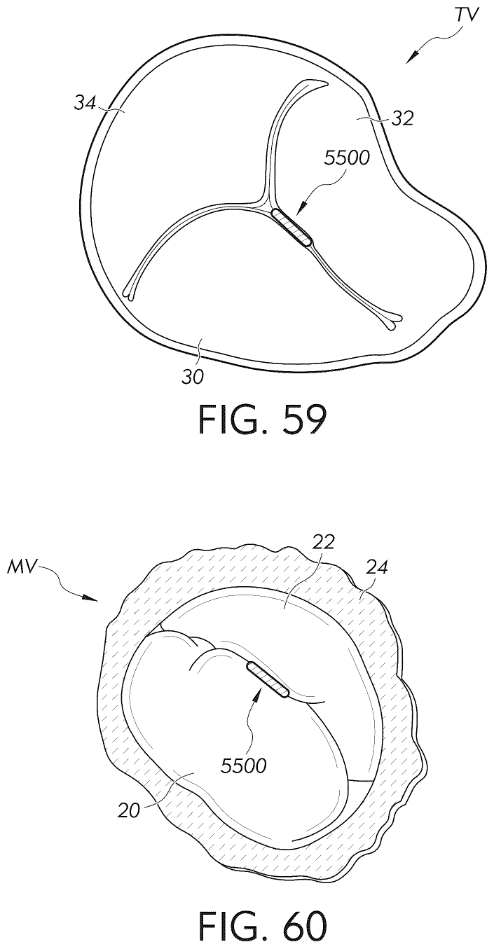

In the example of FIGS. 56-58, the tricuspid valve TV is illustrated as being repaired at, or proximate, the commissure of the anterior leaflet 30 and the septal leaflet 32 of the tricuspid valve TV. The device 5500, however, can be positioned at any desired location between the native leaflets. For example, as shown in FIG. 59, the device 5500 can be placed between mid-portions of the native leaflets (e.g., the anterior leaflet 30 and the septal leaflet 32 of the tricuspid valve TV) resulting in these mid-portions clamped together. While FIGS. 56-59 illustrate repair of the tricuspid valve TV, the device 5500 can be used to repair other valves, such as the mitral valve MV. For example, as shown in FIG. 60, the device 5500 can be positioned in the middle of the mitral valve MV resulting in these mid-portions clamped together (or can be placed at the commissure regions to restrict the remaining open area through which blood can flow).

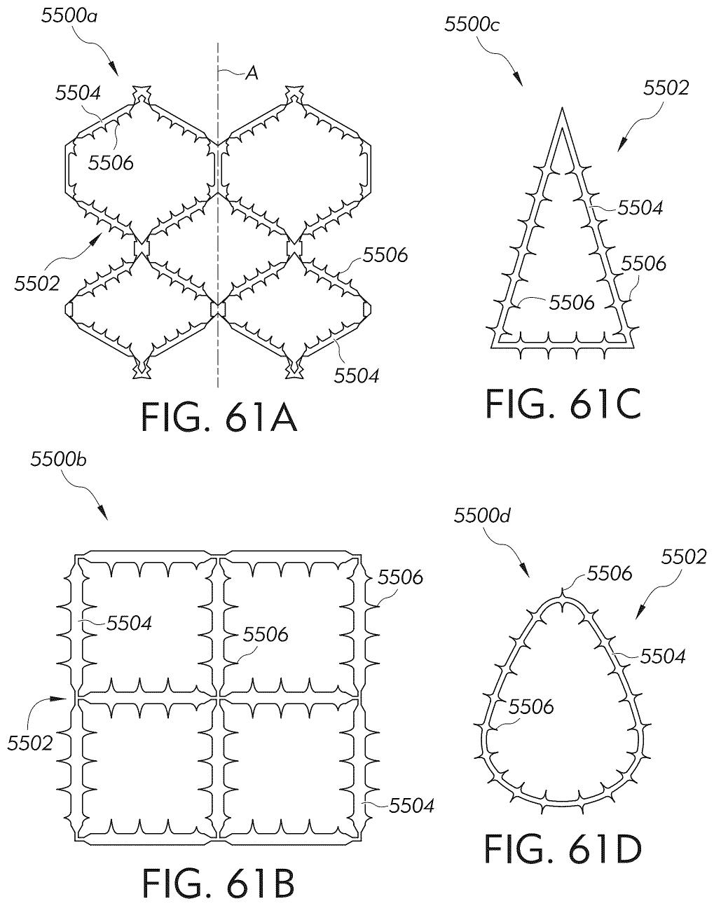

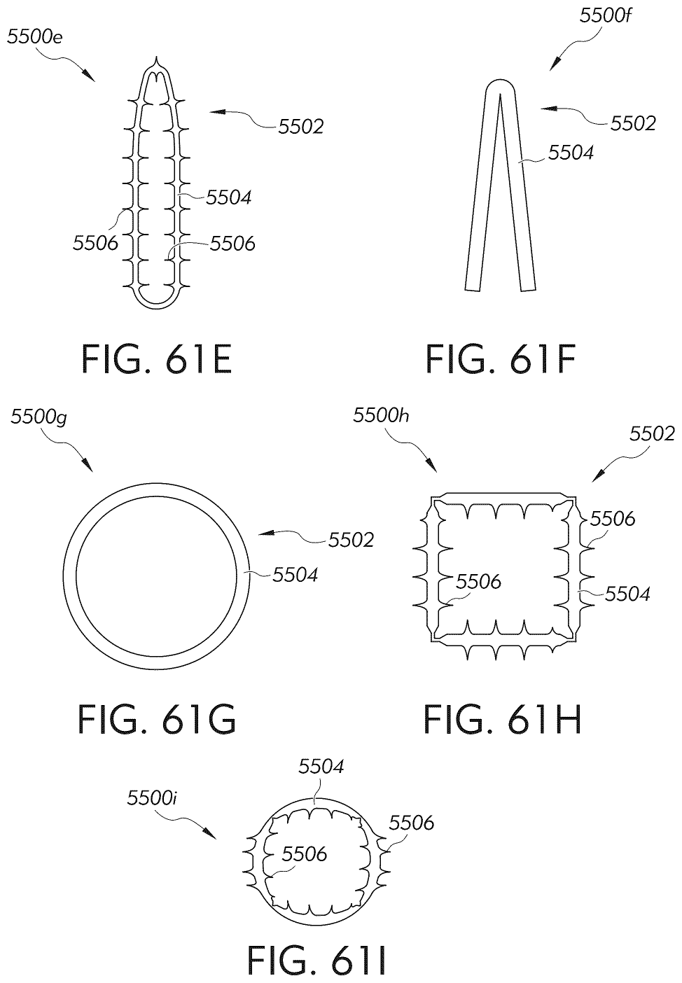

FIGS. 61A-61I illustrate side views of various implementations of the device 5500. Referring to the example of FIG. 61A, the device 5500A includes a thin, planar frame 5502. In reference to FIG. 61A, “thin” refers to a depth into the page with the page defining a plane and “thin” meaning a depth that is less than a width or a length of the device, such as less than 50%, such as less than 25%, such as less than 10%, such as less than 5%, or such as less than 2% of the length or width of the device.

The frame 5502 can include a plurality of interconnected struts 5504. The number and arrangement of the struts 5504 can vary in different implementations. For example, the struts 5504 can be arranged in different geometric shapes (e.g., circles, triangles, diamonds, squares, rectangles, pedals, ovals, etc.). Each frame 5502 can have one or more of the same shape or different shapes within the same frame 5502. In the illustrated implementation of FIG. 61A, the struts 5504 are arranged in diamond, rhombus, or hexagon shapes. In some implementations, the frame can have a 2×2 pattern with two diamond, rhombus, or hexagon shapes per row and per column.

Each of the struts 5504 can be configured for attaching to leaflet tissue. The struts can be configured to attach to leaflet tissue in a variety of ways. For example, the struts can have structure configured to grasp the leaflet tissue, such as barbs, hooks, spikes, clasps, etc. In the illustrated example, the struts 5504 include a plurality of spikes 5506 extending outward from the struts 5504 coplanar with the plane of the struts 5504 or the frame 5502. Each strut 5504 can include any suitable number of spikes 5506 arranged along the sides of the struts 5504. Each strut 5504 can include spikes 5506 extending from both sides of the strut 5504 or from only one side of the strut 5504.

The frame 5502 of the device 5500A can be made of any suitable material(s). In some implementations, the frame 5502 can be formed from a metal sheet or tube, wherein the spikes 5506 are cut (e.g., by laser cutting) to extend from respective struts 5504 along the same plane of the struts 5504 or frame 5502.

Referring to the example of FIG. 61B, the device 5500B is substantially similar to the device 5500A in that the device 5500B includes a thin, planar frame 5502 that includes a plurality of interconnected struts 5504. Each of the struts 5504 can be configured for attaching to leaflet tissue, such as with a plurality of spikes 5506 extending outward from the struts 5504 coplanar with the plane of the struts 5504 or the frame 5502. Each strut 5504 can include any suitable number of spikes 5506 arranged along the sides of the struts 5504. Each strut 5504 can include spikes 5506 extending from both sides of the strut 5504 or from only one side of the strut 5504. For the device 5500B, however, the struts 5504 are arranged in square shapes. In particular, the device 5500B has a 2×2 pattern with two square shapes per row and per column.

Referring to the example of FIG. 61C, the device 5500C is substantially similar to the device 5500A in that the device 5500C includes a thin, planar frame 5502 that includes a plurality of interconnected struts 5504. Each of the struts 5504 can be configured for attaching to leaflet tissue. For example, the struts can have a plurality of spikes 5506 extending outward from the struts 5504 coplanar with the plane of the struts 5504 or the frame 5502. Each strut 5504 can include any suitable number of spikes 5506 arranged along the sides of the struts 5504. Each strut 5504 can include spikes 5506 extending from both sides of the strut 5504 or from only one side of the strut 5504. For the device 5500C, the struts 5504 are arranged in a triangular shape.

Referring to the example of FIG. 61D, the device 5500D is substantially similar to the device 5500A in that the device 5500D includes a thin, planar frame 5502 that includes a plurality of interconnected struts 5504. Each of the struts 5504 can be configured for attaching to leaflet tissue. For example, the struts can include a plurality of spikes 5506 extending outward from the struts 5504 coplanar with the plane of the struts 5504 or the frame 5502. Each strut 5504 can include any suitable number of spikes 5506 arranged along the sides of the struts 5504. Each strut 5504 can include spikes 5506 extending from both sides of the strut 5504 or from only one side of the strut 5504. For the device 5500D, however, the struts 5504 are arranged in an egg shape.

Referring to the example of FIG. 61E, the device 5500E is substantially similar to the device 5500A in that the device 5500E includes a thin, planar frame 5502 that includes a plurality of interconnected struts 5504. Each of the struts 5504 can be configured for attaching to leaflet tissue. For example, the struts can include a plurality of spikes 5506 extending outward from the struts 5504 coplanar with the plane of the struts 5504 or the frame 5502. Each strut 5504 can include any suitable number of spikes 5506 arranged along the sides of the struts 5504. Each strut 5504 can include spikes 5506 extending from both sides of the strut 5504 or from only one side of the strut 5504. For the device 5500E, however, the struts 5504 are arranged in a narrow oval shape.

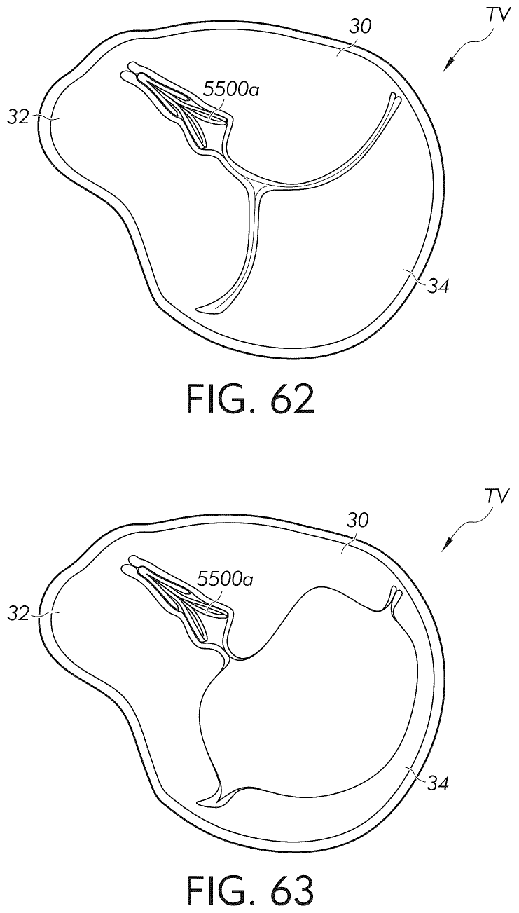

Referring to the example of FIG. 61F, in some implementations the frame 5502 can be shaped to form a device 5500F having a three-dimensional shape that is configured to engage the native valve leaflets. The frame 5502 can be shaped in a variety of different ways. For example, the frame 5502 can be bent, molded, formed, etc. The frame 5502 can be shaped to conform to the shape of a portion of the native valve. For example, the frame 5502 can be bent into the V-shape illustrated by FIG. 61F to engage a commissure portion of the native mitral valve or the native tricuspid valve (see FIGS. 62-64). The V-shape of the frame 5502 corresponds to the general V-shape of the commissures of the native valves. Any of the frames 5502 disclosed herein can be shaped to conform to a portion of the native valve. The optional spikes are not shown on the device 5500F illustrated by FIG. 61F but can be included.

Referring to the example of FIG. 61G, the device 5500G is substantially similar to the device 5500A in that the device 5500G includes a thin, planar frame 5502 that includes a strut 5504. The strut 5504 can be configured for attaching to leaflet tissue. For example, the strut can include a plurality of spikes (not shown but substantially similar to spikes 5506 of the device 5500D, for example). The spikes can extend outward from the strut 5504 coplanar with the plane of the struts 5504 or the frame 5502. The strut 5504 can include any suitable number of spikes arranged along the sides of the strut 5504. The strut 5504 can include spikes extending from both sides of the strut 5504 or from only one side of the strut 5504. For the device 5500F the strut 5504 is formed in an annular shape.

Referring to the example of FIG. 61H, the device 5500H is substantially similar to the device 5500A in that the device 5500H includes a thin, planar frame 5502 that includes a plurality of interconnected struts 5504. Each of the struts 5504 can be configured for attaching to leaflet tissue. For example, the struts can include a plurality of spikes 5506 extending outward from the struts 5504 coplanar with the plane of the struts 5504 or the frame 5502. Each strut 5504 can include any suitable number of spikes 5506 arranged along the sides of the struts 5504. Each strut 5504 can include spikes 5506 extending from both sides of the strut 5504 or from only one side of the strut 5504. For the device 5500H, however, the struts 5504 are arranged in a square shape.

Referring to the example of FIG. 61I, the device 5500I is substantially similar to the device 5500A in that the device 5500I includes a thin, planar frame 5502 that includes a plurality of interconnected struts 5504. Each of the struts 5504 can be configured for attaching to leaflet tissue. For example, the struts can include a plurality of spikes 5506 extending outward from the struts 5504 coplanar with the plane of the struts 5504 or the frame 5502. Each strut 5504 can include any suitable number of spikes 5506 arranged along the sides of the struts 5504. Each strut 5504 can include spikes 5506 extending from both sides of the strut 5504 or from only one side of the strut 5504. For the device 5500I however, the struts 5504 are arranged in a circular shape.

Referring to FIGS. 62-64, the device 5500A is illustrated installed in the tricuspid valve TV. The device 5500A is installed in a shaped configuration, such as the V shaped configuration illustrated by FIG. 61F. In some implementations, the device 5500A is folded along a mid-line A (FIG. 61A) in positioned at the commissure of the anterior leaflet 30 and the septal leaflet 32. In other implementations, the device 5500A, or any of the devices 5500 disclosed herein, can be folded.

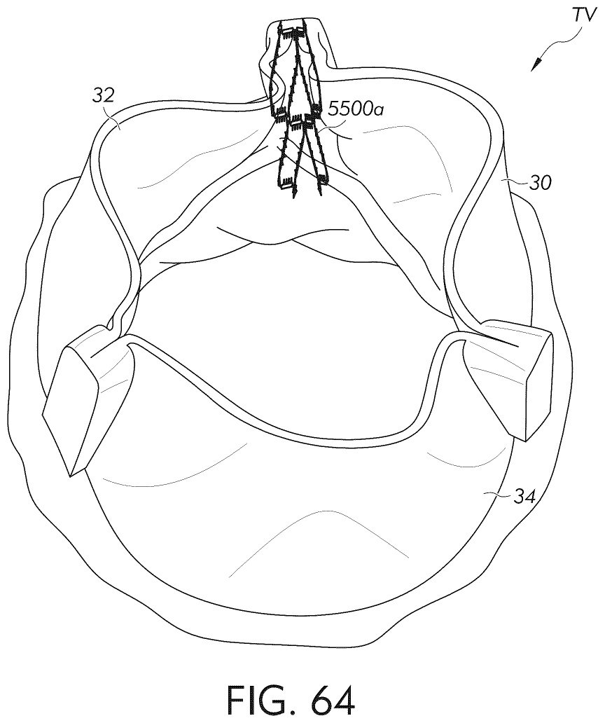

FIG. 62 illustrates the tricuspid valve TV in the systolic phase, or systole, with the leaflets 30, 32, 34 closed. In systole, the tissue of the anterior leaflet 30 and the septal leaflet 32 adjacent the device 5500A attach to the device 5500A, as shown in FIG. 62. For example, the spikes 5506 can pierce or penetrate into the leaflet tissue when the leaflets 30, 32 come into contact with the spikes 5506. As a result, the leaflet tissue becomes attached to the device 5500A. During the subsequent diastolic phase, as shown in FIGS. 63-64, the leaflets 30, 32, 34 open, but the portion of the anterior leaflet 30 and the septal leaflet 32 that are attached to the device 5500A are held in the closed condition by the device 5500A. In this manner, the device can reduce the effective size of a regurgitant valve and/or close a regurgitant area of the valve to reduce regurgitant flow through the valve.

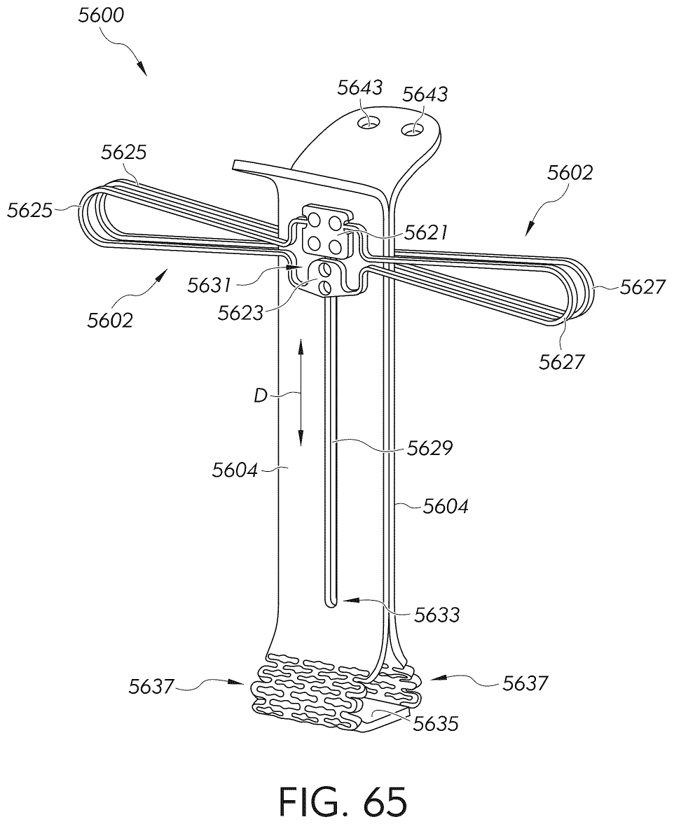

FIGS. 65-75 illustrate a device or implant 5600 (e.g., a prosthetic device, a valve repair device, implantable device, etc.) useable for repairing a native heart valve (e.g., the mitral valve or the tricuspid valve). The device or implant 5600 can include any of the features for another device or implant discussed in the present application or the applications cited above, and the device 5600 can be positioned to engage chordae CT and/or valve tissue (e.g., leaflets 20, 22, 30, 32, 34) as part of any suitable valve repair system (e.g., any valve repair system disclosed in the present application, or the applications cited above).

The device or implant 5600 can be deployed from a delivery system (e.g., delivery system 102). The delivery system can comprise one or more of a catheter, a sheath, a guide catheter/sheath, a delivery catheter/sheath, a steerable catheter, an implant catheter, a tube, a channel, a pathway, combinations of these, etc.

In some implementations, the device or implant 5600 can be configured to restore leaflet coaptation via chordae approximation and/or leaflet approximation. In some implementations, the device or implant 5600 can include a one or more paddles 5604 and one or more capture elements 5602.

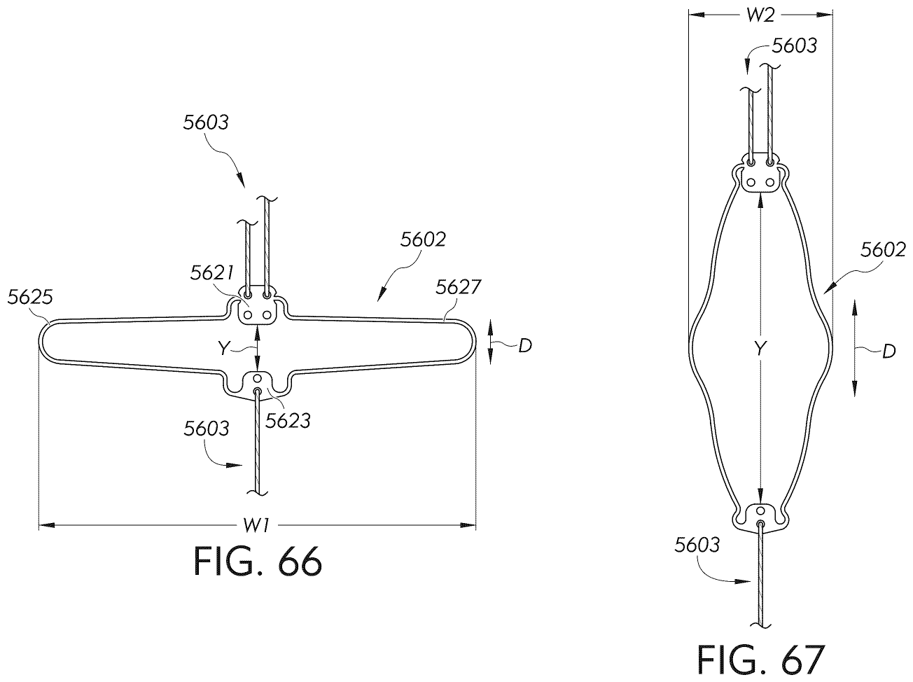

The capture element(s) 5602 can optionally be configured to move between an expanded configuration (as shown in FIGS. 65-66) and an elongated configuration (as shown in FIG. 67), where the capture elements 5602 are configured to engage tissue (e.g., leaflets, chordae tendineae CT (e.g., the primary chordae (closest to the edge of the leaflet)) of two or more leaflets, etc.) to pull the leaflets together when in the expanded configuration; thus, reducing the gap between the leaflets.

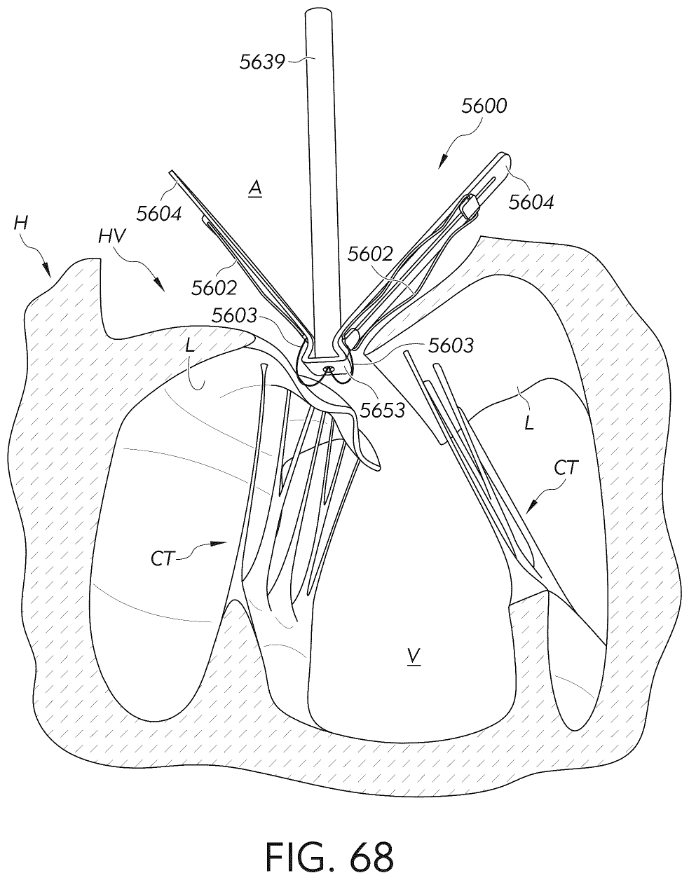

In some implementations, the paddle(s) 5604 are moveable between an open configuration (as shown in FIGS. 68-70) and a closed configuration (as shown in FIGS. 65 and 71-75). In some implementations, the movement of the paddle(s) 5604 from the open configuration to the closed configuration allows for the capture elements 5602 to engage tissue (e.g., to engage leaflets to pull the leaflets together, to engage the chordae tendineae CT to pull the leaflets together, etc.).

In some implementations, one or more actuation element 5603 (FIGS. 66-67) can allow a user to engage the capture element(s) 5602 to move the capture element(s) 5602 between the expanded and elongated configurations.

In some implementations, the capture element 5602 can be configured to engage multiple chordae tendineae CT to distribute the tension force between multiple chordae.

Referring to FIGS. 65-67, the fixation element or capture element 5602 can include a first connection element 5621, a second connection element 5623, a first flexible portion 5625, and a second flexible portion 5627. In some implementations, the first and second flexible portions 5625, 5627 are each connected to both of the first and second connection elements 5621, 5623 such that movement of one or both of the first and second connection elements 5621, 5623 causes the flexible portions 5625, 5627 to flex and move the capture element 5602 between the elongated configuration (as shown in FIG. 67) and the expanded configuration (as shown in FIG. 66). For example, referring to FIGS. 66-67, movement of one or both of the connection elements 5621, 5623 in the directions D causes the distance Y between the connection elements 5621, 5623 to be adjusted and, consequently, causes the flexible portions 5625, 5627 to flex.

In some implementations, the first connection element 5621, the second connection element 5623, the first flexible portion 5625, and the second flexible portion 5627 are integrally formed. The first connection element 5621, the second connection element 5623, the first flexible portion 5625, and the second flexible portion 5627 can be made from any suitable material(s), such as, for example, spring materials, such as steel, other metals, plastics, composites, etc. or shape-memory alloys such as Nitinol, or any combination thereof. The capture elements 5602 can be configured in a variety of ways, including different cross-sectional shapes, different sizes (e.g., length, width, thickness), composed of different materials, etc.

In some implementations, the first and second connection elements 5621, 5623 can be connected to a paddle 5604, with at least one of the connection elements 5621, 5623 being movably connected to the paddle 5604, such that one or more of the connection elements 5621, 5623 can be moved in the directions D to adjust the distance Y between the connection elements 5621, 5623. This adjustability of the distance Y between the connection elements 5621, 5623 allows for the capture element 5602 to be moved between the elongated and expanded configurations. In some implementations, one or more of the connection elements 5621, 5623 can be movably connected to a slot 5629 (FIG. 65) of the paddles 5604 such that the one or more connection elements 5621, 5623 are movable (e.g., slidable) within the slot 5629 to adjust the distance Y between the connection elements 5621, 5623. One or both of the connection elements 5621, 5623 can be movably connected to the slot 5629 by a pin and slot connection or any other suitable connection.

Referring to FIGS. 65 and 66, in some instances, the capture elements 5602 can normally be (e.g., biased or shape set) in the expanded configuration such that the distance Y between the connection elements 5621, 5623 is minimal. In some implementations, the actuation element(s) 5603 can be engaged by a user to cause one or more of the connection elements 5621, 5623 in the direction D such that the distance Y increases and the capture elements 5602 move to the elongated configuration. Releasing of the actuation element(s) 5603 by the user allows for one or both of the connection elements 5621, 5623 to move in the direction D such that the distance Y between the connection elements 5621, 5623 decreases and the capture elements 5602 move back to the normal, expanded configuration. In other instances, the capture elements 5602 can be in a normally, elongated configuration and engagement of one or more of the connection elements 5621, 5623 (e.g., via the actuation element(s) 5603) causes the capture elements 5602 to move to the expanded configuration. The capture elements 5602 can be in the normally expanded configuration or the normally elongated configuration by being shape set in such a configuration.

Referring to FIG. 65, the slot 5629 can include a top end 5631 and a bottom end 5633. In some instances, the first connection element 5621 can be fixed proximate the top end 5631 and the second connection element 5623 can be movably attached to the slot 5629 such that movement of the second connection element 5623 in the directions D causes the capture element 5602 to move between the expanded and elongated configurations. In some instances, both of the first and second connection elements 5621, 5623 can be movably connected to the slot 5629 such that one or both of these connection elements 5621, 5623 can be moved in the direction D to move the capture element 5602 between the expanded and elongated configurations, and such that the capture element 5602 can be in the expanded configuration at any location along the height of the slot 5629.

Referring to FIGS. 66-67, in some implementations, a ratio of the width W1 (FIG. 66) of the capture elements 5602 when in the expanded configuration to the width W2 (FIG. 67) of the capture elements 5602 when in the elongated configuration can be between 20/1 and 2/1, such as between 15/1 and 3/1, such as between 10/1 and 4/1 or any subrange of these ranges. In some examples, the width W1 can be between 5 mm and 25 mm, such as between 7 mm and 20 mm, such as between 15 mm and 20 mm or any subrange of these ranges. In some examples, the width W2 can be between 1 mm and 10 mm, such as between 2 mm and 5 mm, such as between 3 mm and 4 mm, or any subrange of these ranges.

Still referring to FIG. 65, in some implementations, the device or implant 5600 can include two or more paddles 5604 that are connected to an optional base member 5635 by hinge portions 5637. In some implementations, the base member 5635 is omitted and the paddles 5604 are connected by a hinge portion 5637. The hinge portions 5637 allow for the paddles 5604 to pivot relative to the base member 5635 to move the paddles 5604 between the open and closed configurations. The paddles 5604, the hinge portions 5637, and the base member 5635 can be made of, for example, spring materials, such as steel, other metals, plastics, composites, etc. or shape-memory alloys such as Nitinol, or any combination thereof.

In some implementations, the paddles 5604, the hinge portions 5637, and the base member 5635 can be integrally formed. In some examples, the device or implant 5600 can be configured such that the paddles 5604 are normally in a closed configuration (e.g., by being made of a shape-set material) and an actuation element (e.g., actuation catheter 5639 shown in FIGS. 68-71, a tube, a wire, and/or a suture, etc.) of a delivery system can be configured to engage the device or implant 5600 to move the paddles 5604 to the open configuration.

In some implementations, the paddles 5604 can include openings 5643 for receiving a tube, a wire, and/or a suture, or other type of actuation element that is capable of moving the paddles 5604 between the open and closed configurations.

In some implementations, an actuation catheter 5639 can be configured to engage hinge portions 5637 to move the paddles 5604 between the open and closed configurations. It should be understood, however, that the paddles 5604 can take any suitable form, such as, for example the form of any type of mechanical clip, and it should be understood that the paddles 5604 can be moved between the open and closed configurations by any suitable means, such as, for example, by using any type of mechanical feature that allows the paddles 5604 to be moved between open and closed configurations.

Referring to FIGS. 68-71, the device 5600 is shown being delivered and deployed within the native heart valve HV (e.g., the tricuspid valve, the mitral valve, etc.) of the heart H. Referring to FIG. 68, a delivery sheath/catheter (not shown) can be inserted into the atrium A of the heart H and the implant/device 5600 and an actuation element (e.g., an actuation catheter or tube, a wire, a suture etc.) can be deployed from the delivery catheter/sheath. While the device 5600 is shown being deployed with an actuation catheter 5639, it should be understood that the device can be deployed with any other type of actuation element that is capable of moving the paddles 5604 between the open and closed configurations, such as, for example, any actuation element described in the present application. The device 5600 is shown with the paddles 5604 in the open configuration and the capture elements 5602 in the elongated configuration. In some implementations, the paddles are in the closed position when passing through the native valve and the capture elements 5602 are in the elongated configuration to allow the device to more easily pass through the native valve. The capture elements 5602 in the elongated configuration can allow for the device 5600 to fit within the delivery sheath/catheter.

In some examples, the actuation element 5639 can engage the hinge portions 5637 (FIG. 65) of the device 5600 to cause the paddles to flex outward to the open configuration when deployed from the delivery catheter/sheath. The device 5600 can optionally be configured to be compressed when within the delivery catheter/sheath such that the device 5600 is in the closed configuration prior to deployment from the delivery catheter/sheath. In some examples, removal of the actuation element 5639 from the base and/or retraction of the actuation element 5639 into the delivery catheter/sheath can cause the paddles to move back to a normally closed configuration. The actuation element 5639 can be removably connected to the base 5635 by any suitable type of removable connection, such as, for example, threaded connection. However, it should be understood that the device 5600 can take any suitable form (such as any form described in the present application) and be in an open or closed configuration when deployed from the delivery catheter/sheath. It should also be understood that the actuation element 5639 can engage the device 5600 in any suitable manner that causes the device to move between the open and closed configurations, such as, for example, any manner described in the present application.

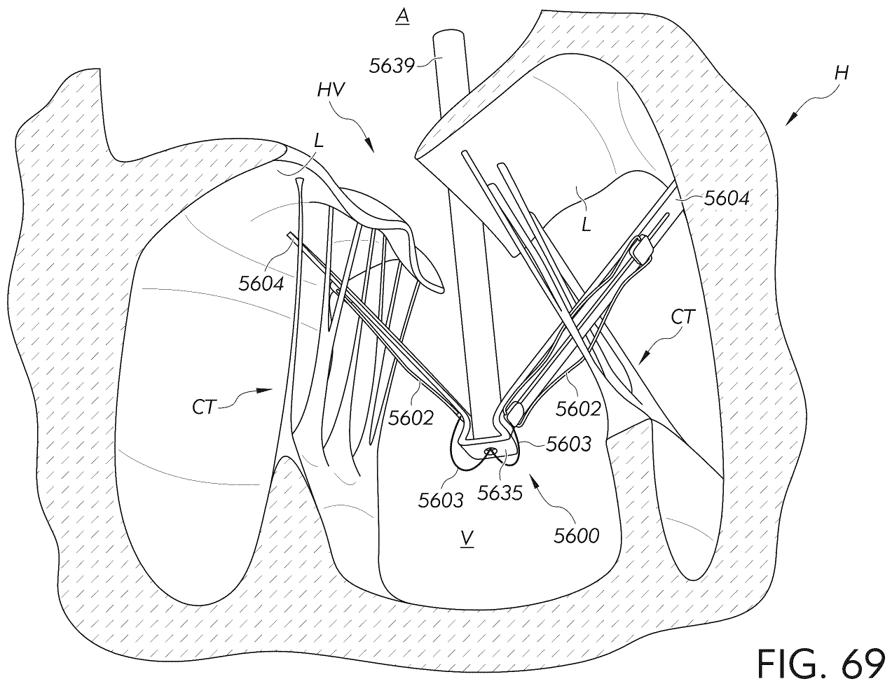

Referring to FIG. 69, the device 5600 is moved into position through the heart valve HV and into the ventricle V with the paddles 5604 in the open configuration and the capture elements 5602 in the elongated configuration. The capture elements 5602 being in the elongated configuration allow for the paddles 5604 and capture elements 5602 to extend through and behind chordae tendineae CT without pushing the chordae tendineae CT outward.

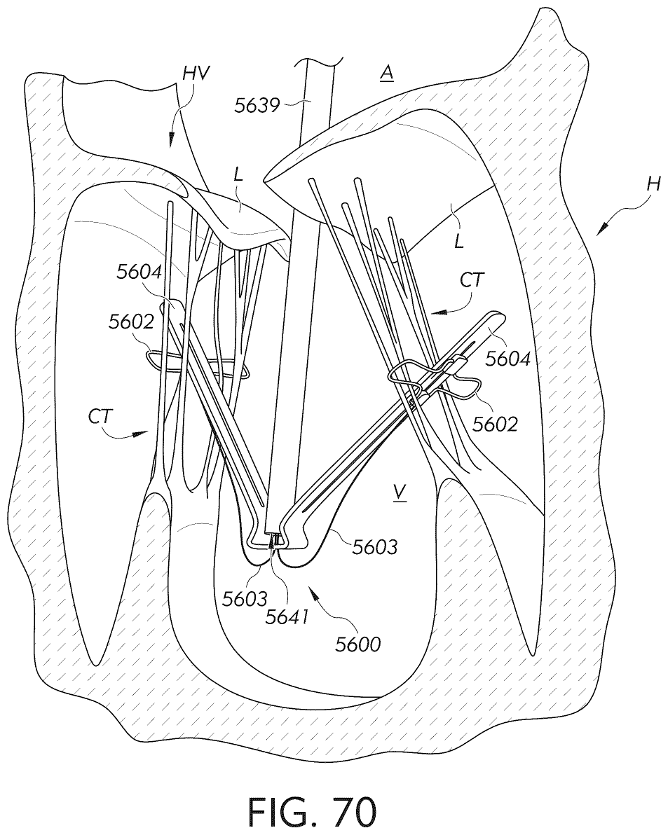

Referring to FIG. 70, in some implementations, once the capture elements 5602 are positioned behind the chordae tendineae CT, the capture elements 5602 can be moved to the expanded configuration such that the capture elements 5602 extend behind and across one or more chordae.

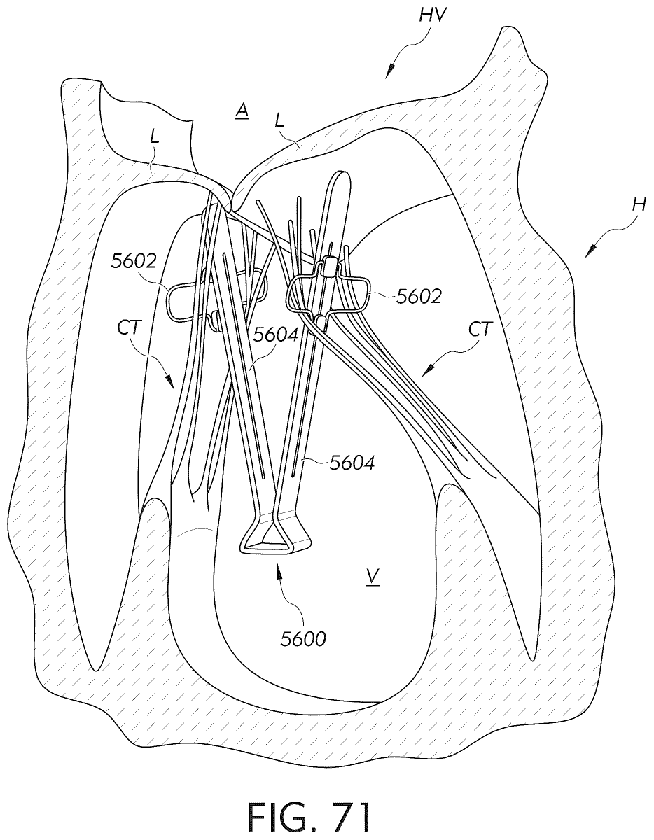

The capture elements 5602 can be moved from the elongated configuration to the expanded configuration by any suitable means, such as, for example, any means described in the present application. In the illustrated example, actuation elements 5603 extend from the distal end 5641 of the actuation element 5639 such that the actuation elements 5603 engage connection portions 5621, 5623 (FIGS. 65-67) to maintain the capture elements 5602 in the elongated configuration (as shown in FIGS. 68-69), and the force provided to the actuation elements 5603 is released to allow the capture elements 5602 to move to their normally expanded configuration (as shown in FIGS. 70-71). It should be understood, however, that the capture elements 5602 can be moved between the elongated and expanded configurations by any suitable means, such as, for example, any means described in the present application.

Referring to FIG. 71, in some implementations, after the capture elements 5602 are positioned behind and extend across one or more chordae tendineae CT, the paddles 5604 can be moved to the closed configuration such that the capture element 5602 engage the chordae tendineae CT of adjacent leaflets L to approximate the chordae and, consequently, approximate the adjacent leaflets L. In some implementations retraction and removal of the actuation element 5639 (FIGS. 68-70) can allow for the paddles 5604 to move to the closed configuration. In some implementations, the capture elements 5602 extend across one or more chordae tendineae CT at and/or near a junction or transition between the chordae tendineae CT and the native valve leaflet.

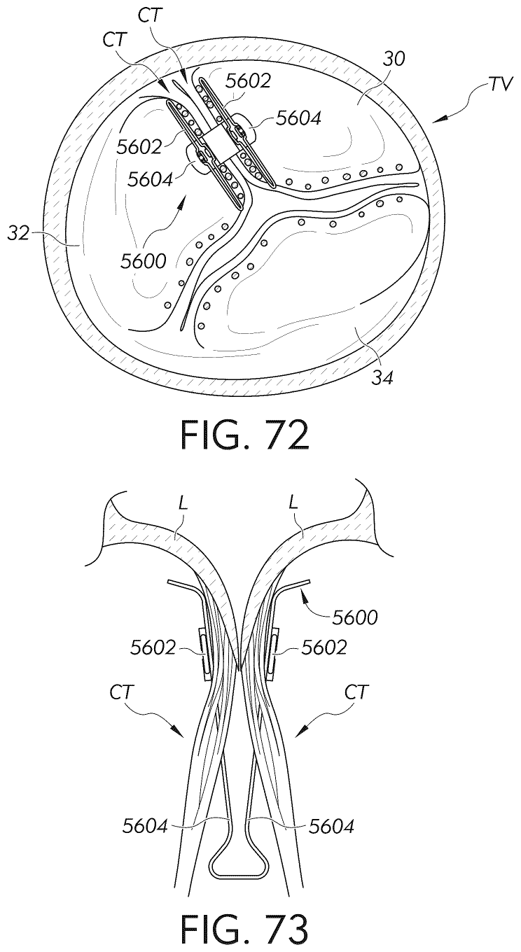

Referring to FIG. 72, the device 5600 is shown with the paddles 5604 in the closed configuration and the capture elements 5602 in the expanded configuration, where the device 5600 engages tissue, such as chordae tendineae CT (e.g., the primary chordae) of each of the anterior leaflet 30 and septal leaflet 32 of the tricuspid valve TV to approximate the leaflets 30, 32 and close a gap therebetween. In some implementations, the device 5600 can approximate the posterior leaflet 34 and an adjacent leaflet of the tricuspid valve TV in a similar manner. While the tricuspid valve TV is illustrated as being repaired, it should be understood that the device 5600 can be used to repair other valves (e.g., the mitral valve). While many examples herein involve capturing chordae tendineae, the principles and features can be similarly used to capture other types of tissue.

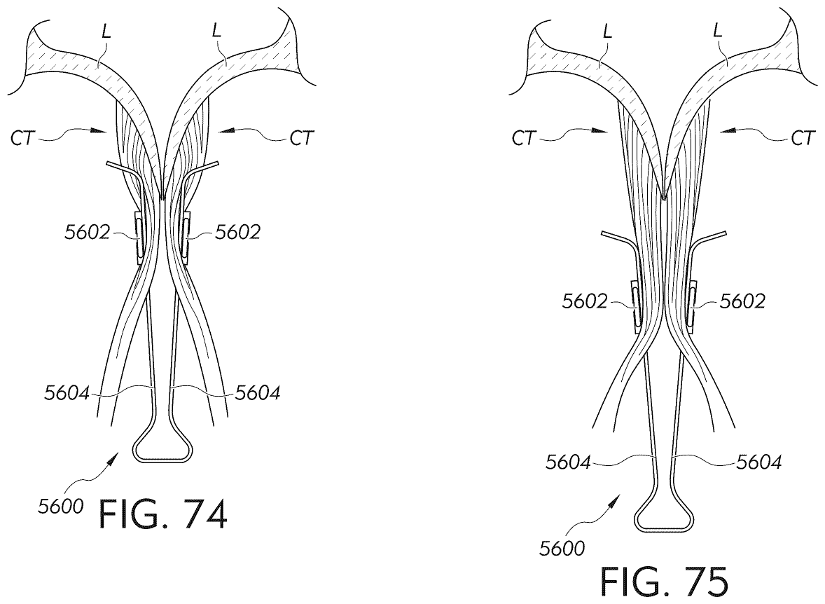

Referring to FIGS. 73-75, the device 5600 can be positioned in a variety of location relative to the leaflets L and chordae tendineae CT to approximate the leaflets L. For example, referring to FIG. 73, the device 5600 can engage the chordae tendineae CT proximate the leaflets L such that the device 5600 also provides a force to the leaflets L when in the closed position. Referring to FIG. 74, in some implementations, the device 5600 can engage the chordae tendineae CT below the leaflets L such that the engagement between the chordae tendineae CT and the device 5600 causes the leaflets L to approximate without the device 5600 providing a force directly to the leaflets L. Referring to FIG. 75, in some implementations, the device 5600 can be positioned to engage the chordae tendineae CT at a location further from the leaflets L (as compared to FIGS. 73-74) such that the engagement between the chordae tendineae CT and the device 5600 causes the leaflets L to approximate without the device 5600 providing a force directly to the leaflets L.

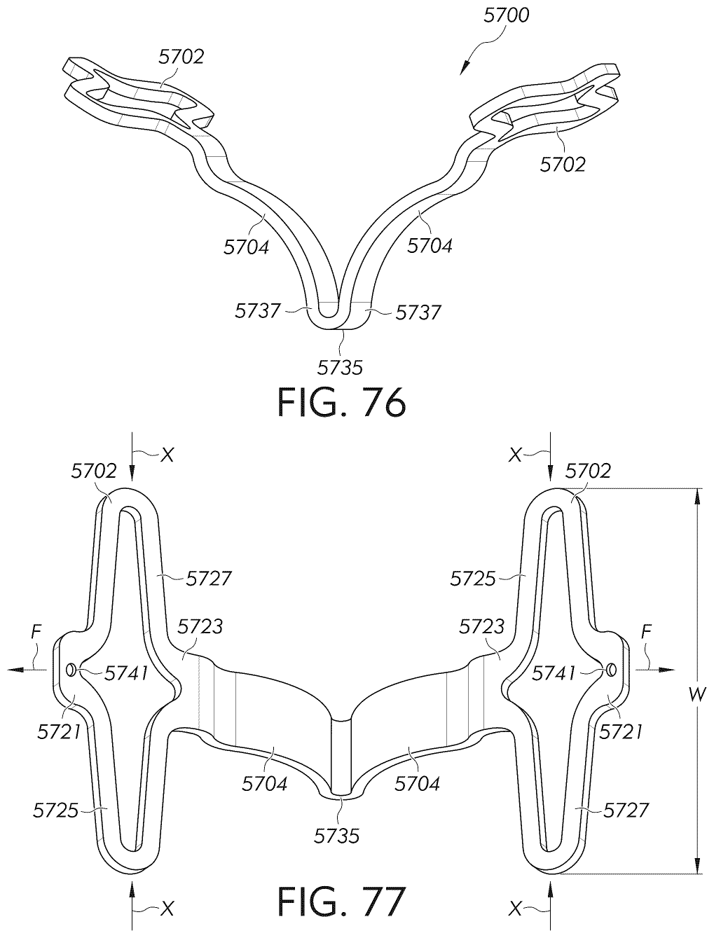

FIGS. 76-77 illustrate a device or implant 5700 (e.g., a prosthetic device, a valve repair device, implantable device, etc.) useable for repairing a native heart valve (e.g., the mitral valve or the tricuspid valve). The device 5700 is similar to the device 5600 described regarding FIGS. 65-75, and the description of the device 5600 applies equally to the device 5700. In the illustrated example, the device 5700 includes capture elements 5702 that are integrally formed with paddles 5704. The paddles 5704 and capture elements 5702 can be made from any suitable materials, such as, for example, spring materials, such as steel, other metals, plastics, composites, etc. or shape-memory alloys such as Nitinol, or any combination thereof.

The device or implant 5700 can be deployed from a delivery system (e.g., delivery system 102). The delivery system can comprise one or more of a catheter, a sheath, a guide catheter/sheath, a delivery catheter/sheath, a steerable catheter, an implant catheter, a tube, a channel, a pathway, combinations of these, etc.

In some implementations, the device or implant 5700 can be configured to restore leaflet coaptation, e.g., via chordae approximation, via leaflet approximation, etc. In some implementations, the device or implant 5700 can include a one or more paddles 5604 and one or more capture elements 5702.

In some implementations, the capture element(s) 5702 are configured to move between an expanded configuration (as shown in FIGS. 76-77) and an elongated configuration (not shown—similar to the elongated configuration for the capture elements 5602 shown in FIGS. 67-69).

In some implementations, the capture elements 5702 are configured to engage the chordae tendineae CT (e.g., the primary chordae (closest to the edge of the leaflet)) of two or more leaflets to pull the leaflets together when in the expanded configuration; thus, reducing the gap between the leaflets. While many examples herein involve capturing chordae tendineae, the principles and features can be similarly used to capture other types of tissue.

In some implementations, the paddle(s) 5704 are moveable between an open configuration (as shown in FIGS. 76-77) and a closed configuration (not shown-similar to the closed configuration for the paddles 5604 shown in FIGS. 65 and 71), where the movement of the paddle(s) 5704 from the open configuration to the closed configuration allows for the capture elements 5602 to engage the chordae tendineae CT to pull the leaflets together. In some implementations, the capture element 5702 can be configured to engage multiple chordae tendineae CT to distribute the tension force between multiple chordae to protect the chordae from being overloaded.

Referring to FIG. 77, the capture elements 5702 can include a free end 5721, a fixed end 5723, a first flexible portion 5725, and a second flexible portion 5727. The capture elements 5702 can be configured to be in a normally expanded configuration (as shown in FIGS. 76-77). The fixed end 5723 can be connected to the paddle 5704 such that each corresponding capture element 5702 moves with the corresponding paddle 5704 as the paddles 5704 move between the open and closed configurations. The first and second flexible portions 5725, 5727 are each connected to both of the free and fixed ends 5721, 5723 such that movement of the free end 5721 away from the fixed end 5723 (e.g., via a force F on the free end 5721) causes the flexible portions 5725, 5727 to flex and move the capture element 5702 from the expanded configuration to the elongated configuration. For example, providing a force F to the free end 5721 causes the free end 5721 to move away from the fixed end 5723 such that a distance Y between the free and fixed ends 5721, 5723 increases. This increase in the distance Y causes the flexible portions 5725, 5727 to move in an inward direction X to adjust a width W of the capture elements 5702 and move the capture elements 5702 from the expanded configuration to the elongated configuration. The capture elements 5702 can be configured in a variety of ways, including different cross-sectional shapes, different sizes (e.g., length, width, thickness), composed of different materials, etc.

One or more actuation elements (not shown) can be configured to engage the capture elements 5702 to allow a user to move the capture elements 5702 between the expanded and elongated configurations. For example, the device 5700 can include an opening(s) 5741 for receiving an actuation element (e.g., a suture, a wire, or any other suitable actuation element described in the present application) that allows a user to provide a force F (FIG. 77) on the capture elements 5702 to move the capture elements 5702 to the elongated configuration.

In some implementations, a ratio of the width W of the capture elements 5702 when in the expanded configuration to the width W of the capture elements 5702 when in the elongated configuration can be between 20/1 and 2/1, such as between 15/1 and 3/1, such as between 10/1 and 4/1 or any subrange of these ranges. In some examples, the width W of the capture elements 5702 in the expanded configuration can be between 5 mm and 25 mm, such as between 7 mm and 20 mm, such as between 15 mm and 20 mm or any subrange of these ranges. In some examples, the width W of the capture elements 5702 in the elongated configuration can be between 1 mm and 10 mm, such as between 2 mm and 5 mm, such as between 3 mm and 4 mm, or any subrange of these ranges.

In some implementations, the device or implant 5700 can include two or more paddles 5704 that are connected to a base member 5735 by hinge portions 5737 (FIG. 77). The hinge portions 5737 allow for the paddles 5704 to pivot relative to the base member 5735 to move the paddles 5704 between the open and closed configurations. In some implementations, the paddles 5704, the hinge portions 5737, and the base member 5735 can be integrally formed. In some examples, the device or implant 5600 can be configured such that the paddles 5704 are normally in a closed configuration and an actuation element (e.g., actuation catheter 5639 shown in FIGS. 68-71, a tube, a wire, a suture, etc.) of a delivery system can be configured to engage the device or implant 5700 to move the paddles 5704 to the open configuration.

The device 5700 can be delivered and deployed within a native heart valve (e.g., the tricuspid valve, the mitral valve, etc.) in a similar manner to the device 5600 shown in FIGS. 68-71. For example, a delivery sheath/catheter (not shown) can be inserted into the atrium of the heart and the implant/device 5700 and an actuation element (e.g., an actuation catheter or tube, a wire, a suture etc.) can be deployed from the delivery catheter/sheath. The device 5600 can be deployed with the paddles 5704 in the open configuration and the capture elements 5702 in the elongated configuration. In some implementations the paddles 5704 can be normally in the open configuration such that deployment from the delivery system causes the paddles 5704 to move from a compressed position within the delivery system to the normally open configuration. In some implementations the paddles 5704 can be normally in the closed configuration and an actuation element(s) can be used to open the paddles after deployment from the delivery system. The device 5700 can be moved through the native heart valve and into the ventricle with the paddles 5704 in the closed configuration or the open configuration and the capture elements 5702 in the elongated configuration.

In some implementations, the capture elements 5702 being in the elongated configuration allow for the paddles 5704 and capture elements 5702 to extend through and behind the chordae tendineae CT without pushing the chordae tendineae CT outward.

In some implementations, once the capture elements 5702 are positioned behind the chordae tendineae, the capture elements 5702 can be moved to the expanded configuration such that the capture elements 5702 extend behind and across one or more chordae. The capture elements 5702 can be moved from the elongated configuration to the expanded configuration by any suitable means, such as, for example, any means described in the present application.

In some implementations, after the capture elements 5702 are positioned behind and extend across one or more chordae tendineae, the paddles 5704 can be moved to the closed configuration such that the capture element 5702 engage the chordae tendineae of adjacent leaflets to approximate the chordae and, consequently, approximate the adjacent leaflets L.

In some implementations, the device 5700 can capture chordae tendineae of adjacent leaflets of the tricuspid valve (e.g., similar to the device 5600 shown in FIG. 72). However, it should be understood that the device 5700 can be used to repair other valves (e.g., the mitral valve). The device 5700 can be positioned at a variety of locations relative to the leaflets and chordae tendineae of the native heart valve, such as, for example, any of the positions described for device 5600 shown in FIGS. 73-75. Also, while many examples herein involve capturing chordae tendineae, the principles and features can be similarly used to capture and treat other types of tissue.

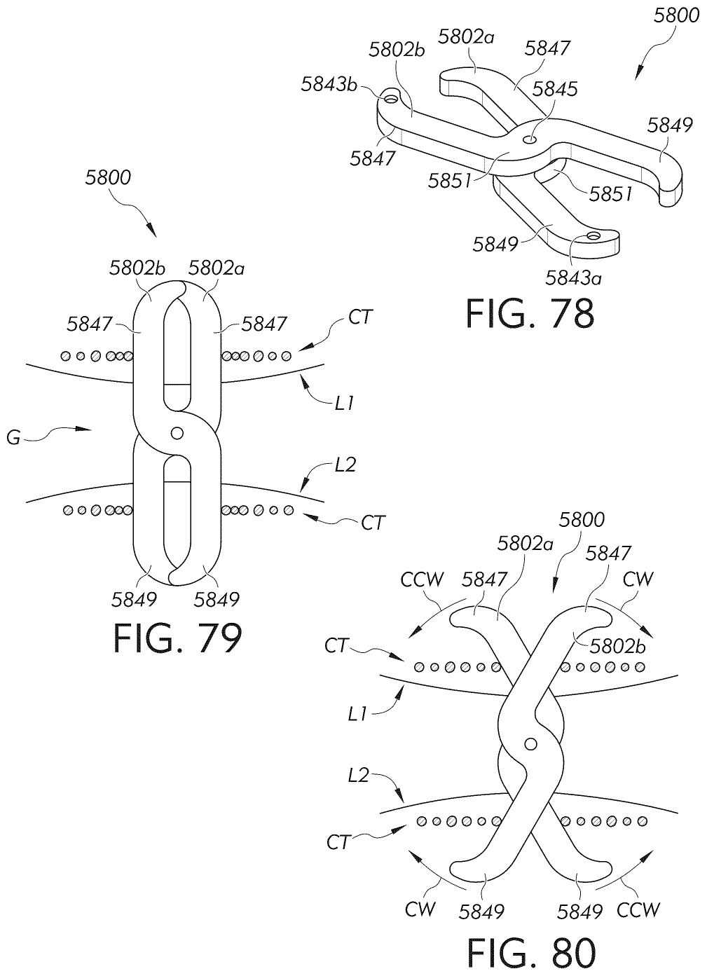

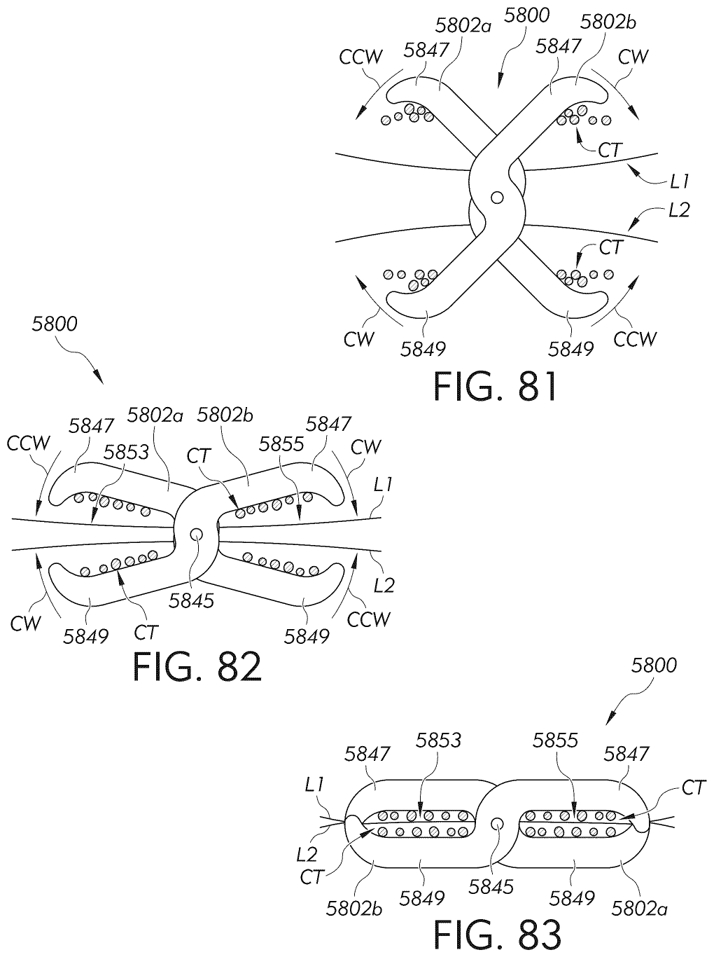

FIGS. 78-84 illustrate a device or implant 5800 (e.g., a prosthetic device, a valve repair device, implantable device, etc.) useable for repairing a native heart valve (e.g., the mitral valve or the tricuspid valve). The device or implant 5800 can include any of the features for another device or implant discussed in the present application or the applications cited above, and the device 5800 can be positioned to engage valve tissue (e.g., leaflets 20, 22, 30, 32, 34) as part of any suitable valve repair system (e.g., any valve repair system disclosed in the present application, or the applications cited above).

The device or implant 5800 can be deployed from a delivery system (e.g., delivery system 102). The delivery system can comprise one or more of a catheter, a sheath, a guide catheter/sheath, a delivery catheter/sheath, a steerable catheter, an implant catheter, a tube, a channel, a pathway, combinations of these, etc. In some implementations, the device or implant 5800 is configured to restore leaflet coaptation, e.g., via chordae approximation, via leaflet approximation, etc.

In some implementations, the device or implant 5800 can include a two or more capture elements 5802a, 5802b that are rotatably connected to each other by a hinge portion 5845 such that the capture elements 5802a, 5802b can be rotated to engage chordae tendineae to restore leaflet coaptation. Each of a first capture element 5802a and a second capture element 5802b can include a first engagement portion 5847, a second engagement portion 5849, and a central portion 5851 connecting the first and second engagement portions 5847, 5849. In some implementations, the first engagement portion 5847, the second engagement portion 5849, and the central portion 5851 for each capture element 5802a, 5802b are formed. The hinge portion 5845 can attach the central portions 5851 of capture elements 5802a, 5802b together. The capture elements 5802a, 5802b can be made of any suitable material, such as, for example, spring materials, such as steel, other metals, plastics, composites, etc. or shape-memory alloys such as Nitinol, or any combination thereof. The hinge portion 5845 can be any suitable type of mechanical hinge, an elastic hinge from the material properties of the capture elements 5802a, 5802b, or any other suitable type of hinge that allows for rotation of the capture elements 5802a, 5802b relative to each other. In some implementations, the capture element 5802a, 5802b can be configured to engage multiple chordae tendineae CT to distribute the tension force between multiple chordae to protect the chordae from being overloaded. The capture elements 5802a, 5802b can be configured in a variety of ways, including different cross-sectional shapes, different sizes (e.g., length, width, thickness), composed of different materials, etc.

The device 5800 can be configured such that rotation of the capture elements 5802a, 5802b in opposite directions relative to each other allow for the first engagement portion 5847 of the first capture element 5802a and second engagement portion 5849 of the second capture element 5802b to move toward each other to create a first capture area 5853 (FIGS. 82-83), and allow for the second engagement portion 5849 of the first capture element 5802a and the first engagement portion 5847 of the second capture element 5802b to move toward each other to create a second capture area 5855 (FIGS. 82-83). In some implementations, the device 5800 is in a delivery position (as shown in FIG. 79) when the first engagement portion 5847 of each of the capture elements 5802a, 5802b are proximate each other and the second engagement portion of each of the capture elements 5802a, 5802b are proximate each other. Rotation of the capture elements 5802a, 5802b in opposite directions cause the device 5800 to move to a capture position (as shown in FIG. 83) in which the first engagement portion 5847 of the first capture element 5802a is proximate the second engagement portion 5849 of the second capture element 5802b and the second engagement portion 5849 of the first capture element 5802a is proximate the first engagement portion 5849 of the second capture element 5802b.

One or more actuation elements (e.g., rods, shafts, sutures, wires, etc.) can be controlled by a user to engage the device 5800 to move the device 5800 from the delivery position (as shown in FIG. 79) to the capture position (as shown in FIG. 83). For example, the first capture element 5802a can have an opening 5843a for receiving an actuation element, and the second capture element 5802b can have an opening 5843b for receiving an actuation element. While the opening 5843a is shown on the second engagement portion 5849 of the first capture element 5802a and the opening 5843b is shown on the first portion 5847 of the second capture element 5802b, it should be understood that the opening 5843a can be on any portion of the first capture element 5802a and the opening 5843b can be on any portion of the second capture element 5802b that allows for the capture elements 5802a, 5802b to be rotated in opposite directions. In some implementations, the one or more actuation elements can include concentric tubes or shafts with one concentric shaft configured to engage the first capture element 5802a and another concentric shaft configured to engage the second capture element 5802b, where rotation of the concentric shafts causes the corresponding capture element 5802a, 5802b to be moved between the delivery and capture positions. While the device 5800 is described as being moved between the delivery position and the capture position via actuation elements being connected to openings 5843a, 5843b, it should be understood that any other type of actuation element that is capable of rotating the capture elements 5802a, 5802b in opposite directions relative to each other can be used.

In some implementations, the device 5800 can be normally in the capture position (e.g., via a spring, a shape set material, etc.) and actuation element(s) can cause the device 5800 to be in the delivery position during delivery of the device 5800. In some implementations, once the device 5800 is positioned to engage the chordae tendineae CT, the actuation element can release the capture elements 5802a, 5802b to allow the device 5800 to move to the capture position. In some implementations, the device 5800 can be normally in the delivery position (e.g., via a spring, a shape set material, etc.) and actuation element(s) can cause the device 5800 to be moved to the capture position once the device 5800 is positioned to engage the chordae tendineae CT. In some implementations, the device 5800 is not normally in the capture or delivery position but is capable of being moved between the capture and delivery positions via engagement by actuation element(s).

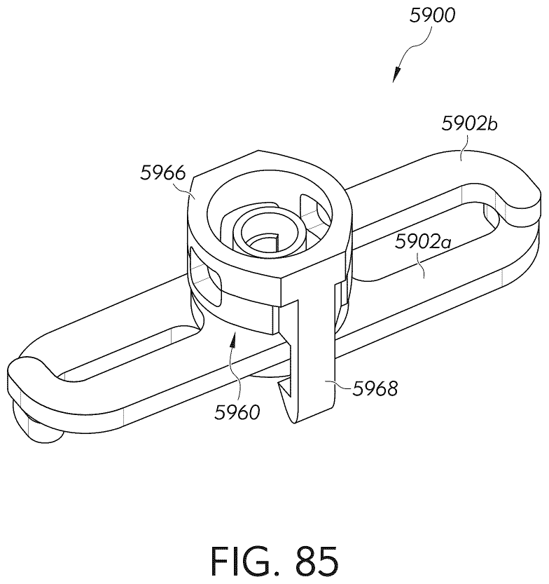

In some implementations, the device 5800 can include one or more locking elements (not shown) that are configured to maintain the device 5800 in the capture position after engaging the chordae tendineae CT. Any suitable locking element that is capable of the device 5800 in the capture position can be used. In some implementations, the locking element can take the form of the clip 5966 and locking extensions 5968 of the device 5900 shown in FIG. 85. However, it should be understood that the locking element can take any other suitable form, such as, for example, a clip, a latch, or any other suitable mechanical lock that is capable of maintaining the capture elements 5802a, 5802b in the capture position.

In some implementations, the capture elements 5802a, 5802b are configured to rotate between about 75 degrees and about 105 degrees to move from the delivery position to the capture position, such as between about 80 degrees and about 100 degrees, such as between about 85 degrees and about 95 degrees, such as about 90 degrees. In some implementations, the capture elements 5802a, 5802b can be limited in the amount of rotation that can be achieved. For example, the capture elements 5802a, 5802b can be configured to rotate 115 degrees or less to move between the delivery position and the capture position, such as 110 degrees or less, such as 105 degrees or less, such as 100 degrees or less, such as 95 degrees or less, such as 90 degrees or less. In some implementations, the capture elements 5802a, 5802b are not limited in the amount of rotation that can be achieved. Each capture elements 5802a, 5802b can be configured to rotate in a single direction to move from the delivery position to the capture position. For example, the first capture element 5802a can be configured to only rotate in a counterclockwise direction and the second capture element 5802b can be configured to only rotate in clockwise direction when moving from the delivery position to the capture position, or vice versa. In some implementations, the capture elements 5802a, 5802b can be configured to rotate in either the clockwise or counterclockwise direction when moving between the delivery position to the capture position. In some implementations, one of the capture elements 5802a, 5802b can be configured to rotate (e.g., about 180 degrees or less) and the other capture element can be fixed.

Referring to FIGS. 79-83, the device 5800 is shown being delivered and deployed within the native heart valve (e.g., the tricuspid valve, the mitral valve, etc.) to engage chordae tendineae CT of adjacent leaflets L1, L2. Referring to FIG. 79, the device 5800 can be moved into a ventricle of the heart with the device 5800 is in the delivery position. The capture elements 5802a, 5802b of the device 5800 is positioned substantially perpendicular to a coaptation gap G separating adjacent leaflets L1, L2.

Referring to FIGS. 79-82, the first capture element 5802a can be rotated in a counterclockwise direction CCW such that the first engagement portion 5847 engages a portion of the chordae tendineae CT connected to one leaflet L1 and the second engagement portion 5849 engages a portion of the chordae tendineae CT connected to the adjacent leaflet L2. The second capture element 5802b can be rotated in a clockwise direction CW such that the first engagement portion 5847 engages a portion of the chordae tendineae CT connected to one leaflet L1 and the second engagement portion 5849 engages a portion of the chordae tendineae CT connected to the adjacent leaflet L2. While the first capture element 5802a is shown being rotated in the counterclockwise direction CCW and the second capture element 5802b is shown being rotated in the clockwise direction CW, it should be understood that the first capture element 5802a can rotated in the clockwise direction CW and the second capture element 5802b can be rotated in the counterclockwise direction CCW. In some implementations, first capture element 5802a can be rotated and the second capture element 5802b can be held stationary.

Referring to FIGS. 82-83, the first engagement portion 5847 of the first capture element 5802a and the second engagement portion 5849 of the second capture element 5802b create a first capture area 5853 for securing a portion of the chordae tendineae CT connected to the adjacent leaflets L1, L2, and the second engagement portion 5849 of the first capture element 5802a and the first engagement portion 5847 of the second capture element 5802b create a second capture area 5855 for securing a another portion of the chordae tendineae CT connected to the adjacent leaflets L1, L2. Referring to FIG. 83, in some implementations, the capture elements 5802a, 5802b can continue rotation in their respective directions until the first engagement portion 5847 of the first capture element 5802a overlaps a portion of the second engagement portion 5849 of the second capture element 5802b, and the second engagement portion 5849 of the first capture element 5802a overlaps a portion of the first engagement portion 5847 of the second capture element 5802b. In some implementations, the capture areas 5853, 5855 are closed by the capture elements 5802a, 5802b to prevent chordae tendineae CT from being released by the device 5800 after implantation.

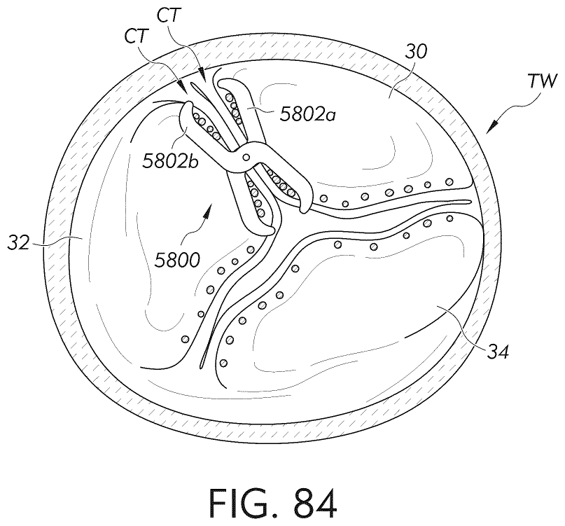

Referring to FIG. 84, the device 5800 is shown with the capture elements 5802a, 5802b in a capture position such that the device 5800 is attached to chordae tendineae CT (e.g., the primary chordae) of each of the anterior leaflet 30 and septal leaflet 32 of the tricuspid valve TV to approximate the leaflets 30, 32 and close a gap therebetween. In some implementations, the device 5800 can be attached to the posterior leaflet 34 and an adjacent leaflet of the tricuspid valve TV in a similar manner. While the tricuspid valve TV is illustrated as being repaired, it should be understood that the device 5800 can be used to repair other valves (e.g., the mitral valve). The device 5800 can be positioned at a variety of locations relative to the leaflets and chordae tendineae of the native heart valve, such as, for example, any of the positions described for device 5600 shown in FIGS. 73-75.

FIGS. 85-89 illustrate a device or implant 5900 (e.g., a prosthetic device, a valve repair device, implantable device, etc.) useable for repairing a native heart valve (e.g., the mitral valve or the tricuspid valve). The device or implant 5900 can include any of the features for another device or implant discussed in the present application or the applications cited above, and the device 5900 can be positioned to engage valve tissue (e.g., leaflets 20, 22, 30, 32, 34) as part of any suitable valve repair system (e.g., any valve repair system disclosed in the present application, or the applications cited above).

The device or implant 5900 can be deployed from a delivery system (e.g., delivery system 102). The delivery system can comprise one or more of a catheter, a sheath, a guide catheter/sheath, a delivery catheter/sheath, a steerable catheter, an implant catheter, a tube, a channel, a pathway, combinations of these, etc. In some implementations, the device or implant 5900 is configured to restore leaflet coaptation, e.g., via chordae approximation, via leaflet approximation, etc.

In some implementations, the device or implant 5900 can include a two or more capture elements 5902a, 5902b and a hub portion 5960 that is attached to the two or more capture elements. 5902a, 5902b. The capture elements 5902a, 5902b can be rotatably connected to each other by a hinge portion (not shown) such that the capture elements 5902a, 5902b can be rotated to engage chordae tendineae and/or leaflets to restore leaflet coaptation. Each of a first capture element 5902a and a second capture element 5902b can include a first engagement portion 5947, a second engagement portion 5949, and a central portion (e.g., similar to central portion 5851 of device 5800 shown in FIGS. 65-75) connecting the first and second engagement portions 5947, 5949. In some implementations, the hub portion 5960 can be connected to the central portion of one or both of the capture elements 5902a, 5902b such that the hub portion 5960 can receive an actuation element (not shown) and position the actuation element to rotate the capture elements 5902a, 5902b. The hinge portion can take any suitable form that allows for the first and second capture elements 5902a, 5902b to rotate relative to each other, such as, for example, any form described for the hinge portion 5845 of device 5800 shown in FIGS. 65-75.

In some implementations, the first engagement portion 5947, the second engagement portion 5949, and the central portion for each capture element 5902a, 5902b are integrally formed. The capture elements 5902a, 5902b can be made of any suitable material, such as, for example, spring materials, such as steel, other metals, plastics, composites, etc. or shape-memory alloys such as Nitinol, or any combination thereof. In some implementations, the capture element 5902a, 5902b can be configured to engage multiple chordae tendineae CT to distribute the tension force between multiple chordae to protect the chordae from being overloaded. The capture elements 5902a, 5902b can be configured in a variety of ways, including different cross-sectional shapes, different sizes (e.g., length, width, thickness), composed of different materials, etc.

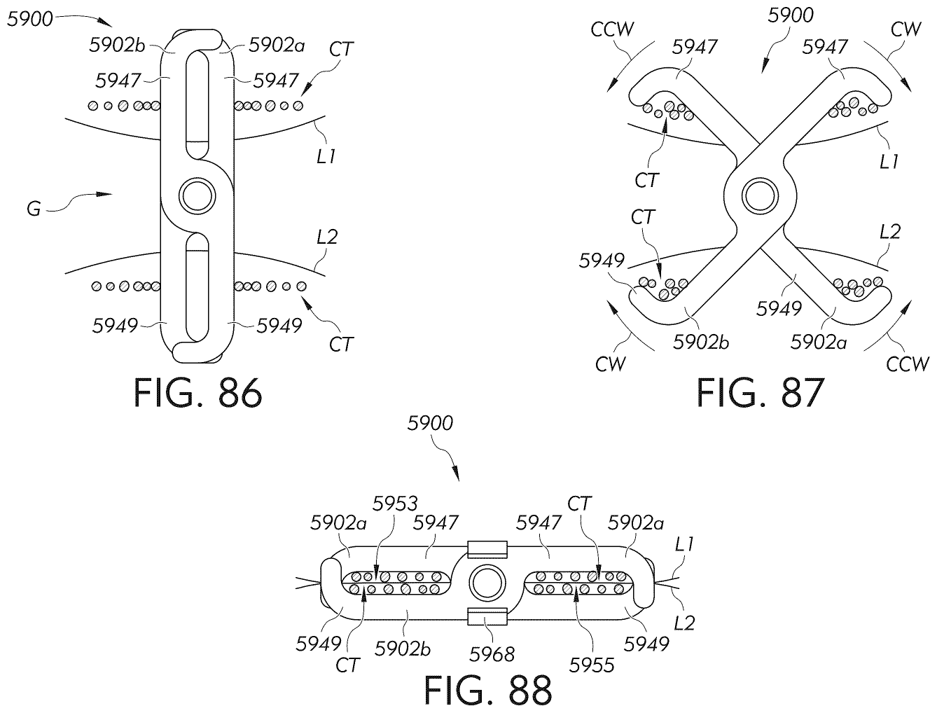

The device 5900 can be configured such that rotation of the capture elements 5902a, 5902b in opposite directions relative to each other allow for the first engagement portion 5947 of the first capture element 5902a and second engagement portion 5949 of the second capture element 5902b to move toward each other to create a first capture area 5953 (FIG. 88), and allow for the second engagement portion 5949 of the first capture element 5902a and the first engagement portion 5947 of the second capture element 5902b to move toward each other to create a second capture area 5955 (FIG. 88). In some implementations, the device 5900 is in a delivery position (as shown in FIG. 86) when the first engagement portion 5947 of each of the capture elements 5902a, 5902b are proximate each other and the second engagement portion of each of the capture elements 5902a, 5902b are proximate each other. Rotation of the capture elements 5902a, 5902b in opposite directions cause the device 5900 to move to a capture position (as shown in FIG. 88) in which the first engagement portion 5947 of the first capture element 5902a is proximate the second engagement portion 5949 of the second capture element 5902b and the second engagement portion 5949 of the first capture element 5902a is proximate the first engagement portion 5949 of the second capture element 5902b.

The hub portion 5960 can be configured to receive one or more actuation elements (not shown) such that the actuation element can engage and rotate the first and second capture elements 5902a, 5902b. In some implementations, the one or more actuation elements can include concentric tubes or shafts with one concentric shaft configured to engage the first capture element 5902a and another concentric shaft configured to engage the second capture element 5902b, where rotation of the concentric shafts causes the corresponding capture element 5902a, 5902b to be moved between the delivery and capture positions. However, the one or more actuation elements can take any other suitable form that is capable of engaging and rotating the capture elements 5902a, 5902b, such as, for example, any form described in the present application.

In some implementations, the device 5900 can be normally in the capture position (e.g., via a spring, a shape set material, etc.) and actuation element(s) can cause the device 5900 to be in the delivery position during delivery of the device 5900. In some implementations, once the device 5900 is positioned to engage the chordae tendineae CT, the actuation element can release the capture elements 5902a, 5902b to allow the device 5900 to move to the capture position.

In some implementations, the device 5900 can be normally in the delivery position (e.g., via a spring, a shape set material, etc.) and actuation element(s) can cause the device 5900 to be moved to the capture position once the device 5900 is positioned to engage the chordae tendineae CT. In some implementations, the device 5900 is not normally in the capture or delivery position but is capable of being moved between the capture and delivery positions via engagement by actuation element(s).

In some implementations, the device 5900 can include one or more locking elements that are configured to maintain the device 5900 in the capture position after engaging the chordae tendineae CT. In the illustrated example, the locking element can be a clip member 5966 that is configured to be secured to the capture elements 5902a, 5902b, where the clip member 5966 includes locking extensions 5968 that are positioned relative to the capture elements 5902a, 5902b when the clip member 5966 is attached to the device 5900 to prevent rotation of the capture elements 5902a, 5902b and, consequently, maintain the capture elements 5902a, 5902b in the capture position. However, it should be understood that any suitable locking element that is capable of maintaining the device 5900 in the capture position can be used. The locking element can be, for example, a clip, a latch, or any other suitable mechanical lock that is capable of maintaining the capture elements 5902a, 5902b in the capture position.

In some implementations, the capture elements 5902a, 5902b are configured to rotate between about 75 degrees and about 105 degrees to move from the delivery position to the capture position, such as between about 80 degrees and about 100 degrees, such as between about 85 degrees and about 95 degrees, such as about 90 degrees. In some implementations, the capture elements 5902a, 5902b can be limited in the amount of rotation that can be achieved. For example, the capture elements 5802a, 5802b can be configured to rotate 115 degrees or less to move between the delivery position and the capture position, such as 110 degrees or less, such as 105 degrees or less, such as 100 degrees or less, such as 95 degrees or less, such as 90 degrees or less. In some implementations, the hub portion 5960 or any other portion of the device 5900 can include one or more stop members (not shown) that are configured to prevent further rotation of a corresponding capture element 5902a, 5902b after the capture element 5902a, 5902b achieves the capture position. In some implementations, the capture elements 5902a, 5902b are not limited in the amount of rotation that can be achieved. In some implementations, one of the capture elements 5902a, 5902b can be configured to rotate (e.g., about 180 degrees or less) and the other capture element can be fixed.

Each capture elements 5902a, 5902b can be configured to rotate in a single direction to move from the delivery position to the capture position. For example, the first capture element 5902a can be configured to only rotate in a counterclockwise direction and the second capture element 5902b can be configured to only rotate in clockwise direction when moving from the delivery position to the capture position, or vice versa. In some implementations, the capture elements 5902a, 5902b can be configured to rotate in either the clockwise or counterclockwise direction when moving between the delivery position to the capture position.

Referring to FIGS. 86-88, the device 5900 is shown being delivered and deployed within the native heart valve (e.g., the tricuspid valve, the mitral valve, etc.) to engage chordae tendineae CT of adjacent leaflets L1, L2. Referring to FIG. 86, the device 5900 can be moved into a ventricle of the heart with the device 5900 is in the delivery position. The capture elements 5902a, 5902b of the device 5900 is positioned substantially perpendicular to a coaptation gap G separating adjacent leaflets L1, L2.

Referring to FIG. 87, the first capture element 5902a can be rotated in a counterclockwise direction CCW such that the first engagement portion 5947 engages a portion of the chordae tendineae CT connected to one leaflet L1 and the second engagement portion 5949 engages a portion of the chordae tendineae CT connected to the adjacent leaflet L2. The second capture element 5902b can be rotated in a clockwise direction CW such that the first engagement portion 5947 engages a portion of the chordae tendineae CT connected to one leaflet L1 and the second engagement portion 5949 engages a portion of the chordae tendineae CT connected to the adjacent leaflet L2. While the first capture element 5902a is shown being rotated in the counterclockwise direction CCW and the second capture element 5902b is shown being rotated in the clockwise direction CW, it should be understood that the first capture element 5902a can rotated in the clockwise direction CW and the second capture element 5902b can be rotated in the counterclockwise direction CCW.

Referring to FIG. 88, the first engagement portion 5947 of the first capture element 5902a and the second engagement portion 5949 of the second capture element 5902b create a first capture area 5953 for securing a portion of the chordae tendineae CT connected to the adjacent leaflets L1, L2, and the second engagement portion 5949 of the first capture element 5902a and the first engagement portion 5947 of the second capture element 5902b create a second capture area 5955 for securing a another portion of the chordae tendineae CT connected to the adjacent leaflets L1, L2. Referring to FIG. 88, in some implementations, the capture elements 5902a, 5902b can continue rotation in their respective directions until the first engagement portion 5947 of the first capture element 5902a overlaps a portion of the second engagement portion 5949 of the second capture element 5902b, and the second engagement portion 5949 of the first capture element 5902a overlaps a portion of the first engagement portion 5947 of the second capture element 5902b. In some implementations, the capture areas 5953, 5955 are closed by the capture elements 5902a, 5902b to prevent or inhibit chordae tendineae CT from being released by the device 5900 after implantation.

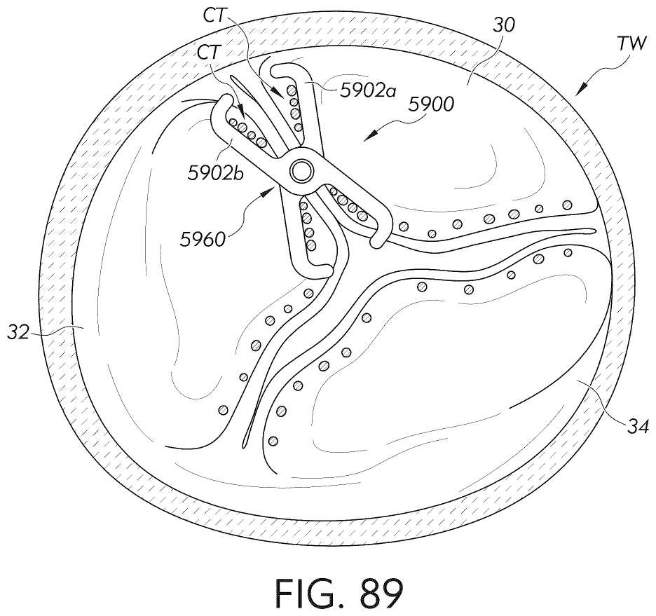

Referring to FIG. 89, the device 5900 is shown with the capture elements 5902a, 5902b in a capture position such that the device 5900 is attached to chordae tendineae CT (e.g., the primary chordae) of each of the anterior leaflet 30 and septal leaflet 32 of the tricuspid valve TV to approximate the leaflets 30, 32 and close a gap therebetween. In some implementations, the device 5900 can be attached to the posterior leaflet 34 and an adjacent leaflet of the tricuspid valve TV in a similar manner. While the tricuspid valve TV is illustrated as being repaired, it should be understood that the device 5900 can be used to repair other valves (e.g., the mitral valve). The device 5900 can be positioned at a variety of locations relative to the leaflets and chordae tendineae of the native heart valve, such as, for example, any of the positions described for device 5600 shown in FIGS. 73-75.

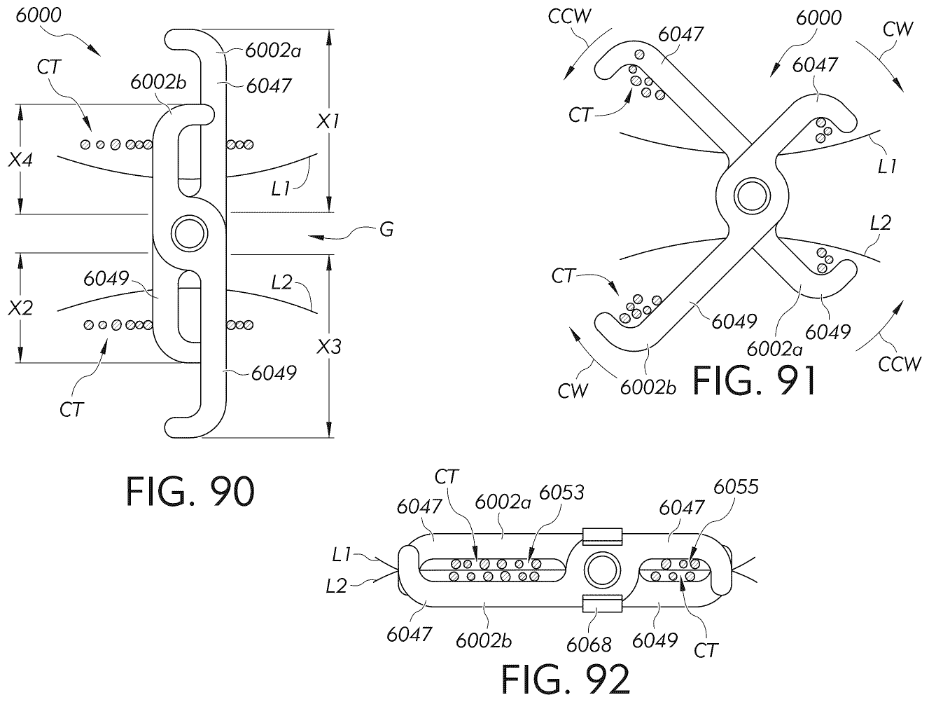

FIGS. 90-93 illustrate a device or implant 6000 (e.g., a prosthetic device, a valve repair device, implantable device, etc.) useable for repairing a native heart valve (e.g., the mitral valve or the tricuspid valve). The device 6000 is similar to the device 5900 described regarding FIGS. 85-89, and the description of the device 5900 applies equally to the device 6000. In the illustrated example, the device 6000 includes capture elements 6002a, 6002b that are similar to the capture elements 5902a, 5902b shown in FIGS. 85-89, except that each of the capture elements 6002a, 6002b include long engagement portions that are configured to create a first capture area 6053 (FIG. 92) and short engagement portions that are configured to create a second capture area 6055 (FIG. 92), where the first capture area 6053 is larger than the second capture area 6055.

In some implementations, the first capture area 6053 can be configured to secure chordae proximate the commissure of a native heart valve, and the second capture area 6055 can be configured to secure chordae proximate a center of the native heart valve. The device or implant 6000 can include any of the features for another device or implant discussed in the present application or the applications cited above, and the device 6000 can be positioned to engage valve tissue (e.g., leaflets 20, 22, 30, 32, 34) as part of any suitable valve repair system (e.g., any valve repair system disclosed in the present application, or the applications cited above).

The device or implant 6000 can be deployed from a delivery system (e.g., delivery system 102). The delivery system can comprise one or more of a catheter, a sheath, a guide catheter/sheath, a delivery catheter/sheath, a steerable catheter, an implant catheter, a tube, a channel, a pathway, combinations of these, etc. The device or implant 6000 is configured to restore leaflet coaptation via chordae approximation.

In some implementations, the device or implant 6000 can include two or more capture elements 6002a, 6002b and a hub portion (not shown) that is attached to the two or more capture elements. 6002a, 6002b. The hub portion can take any suitable form, such as, for example, the form of the hub portion 5960 shown in FIGS. 85-89. The capture elements 6002a, 6002b can be rotatably connected to each other by a hinge portion (not shown) such that the capture elements 6002a, 6002b can be rotated to engage chordae tendineae and/or leaflets to restore leaflet coaptation. Each of a first capture element 6002a and a second capture element 6002b can include a first engagement portion 6047, a second engagement portion 6049, and a central portion (e.g., similar to central portion 5851 of device 5800 shown in FIGS. 65-75) connecting the first and second engagement portions 6047, 6049.

In some implementations, the hub portion can be connected to the central portion of each of the capture elements 6002a, 6002b such that the hub portion can receive an actuation element (not shown) and position the actuation element to rotate the capture elements 6002a, 6002b. In some implementations, the one or more actuation elements can include concentric tubes or shafts with one concentric shaft configured to engage the first capture element 6002a and another concentric shaft configured to engage the second capture element 6002b, where rotation of the concentric shafts causes the corresponding capture element 6002a, 6002b to be moved between the delivery and capture positions. However, the one or more actuation elements can take any other suitable form that is capable of engaging and rotating the capture elements 6002a, 6002b, such as, for example, any form described in the present application. The hinge portion can take any suitable form that allows for the first and second capture elements 6002a, 6002b to rotate relative to each other, such as, for example, any form described for the hinge portion 5845 of device 5800 shown in FIGS. 65-75.

In some implementations, the first engagement portion 6047, the second engagement portion 6049, and the central portion for each capture element 6002a, 6002b are integrally formed. The capture elements 6002a, 6002b can be made of any suitable material, such as, for example, spring materials, such as steel, other metals, plastics, composites, etc. or shape-memory alloys such as Nitinol, or any combination thereof. In some implementations, the capture element 6002a, 6002b can be configured to engage multiple chordae tendineae CT to distribute the tension force between multiple chordae to protect the chordae from being overloaded. The capture elements 6002a, 6002b can be configured in a variety of ways, including different cross-sectional shapes, different sizes (e.g., length, width, thickness), composed of different materials, etc.

The device 6000 can be configured such that rotation of the capture elements 6002a, 6002b in opposite directions relative to each other allow for the first engagement portion 6047 of the first capture element 6002a and second engagement portion 6049 of the second capture element 6002b to move toward each other to create a first capture area 6053 (FIG. 92), and allow for the second engagement portion 6049 of the first capture element 6002a and the first engagement portion 6047 of the second capture element 6002b to move toward each other to create a second capture area 6055 (FIG. 92). In some implementations, the device 6000 is in a delivery position (as shown in FIG. 90) when the first engagement portion 6047 of each of the capture elements 6002a, 6002b are proximate each other and the second engagement portion of each of the capture elements 6002a, 6002b are proximate each other. Rotation of the capture elements 6002a, 6002b in opposite directions cause the device 6000 to move to a capture position (as shown in FIG. 92) in which the first engagement portion 6047 of the first capture element 6002a is proximate the second engagement portion 6049 of the second capture element 6002b and the second engagement portion 6049 of the first capture element 6002a is proximate the first engagement portion 6049 of the second capture element 6002b.

In some implementations, the first engagement portion 6047 of the first capture element 6002a can have a length X1 that is greater than a length X2 of the second engagement portion 6049, and the second engagement portion 6049 of the second capture element 6002b can have a length X3 that is greater than a length X4 of the first engagement portion 6047. In some implementations, the length X1 of the first engagement portion 6047 of the first capture element 6002a is substantially equal to the length X3 of the second engagement portion 6049 of the second capture element 6002b, and the length X2 of second engagement portion 6049 of the first capture element 6002a is substantially equal to the length X4 of the first engagement portion 6047 of the second capture element 6002b. For example, the lengths X1, X3 can be between 5 mm and 20 mm or any subrange. The lengths X2, X4 can be, for example, between 2 mm and 10 mm or any subrange. In some examples, the width of the capture elements 6002a, 6002b in the delivery position can be between 1 mm and 10 mm, such as between 2 mm and 5 mm, such as between 3 mm and 4 mm, or any subrange of these ranges. In other implementations, the length X1 of the first engagement portion 6047 of the first capture element 6002a can be less than the length X2 of the second engagement portion, and the length X3 of the second engagement portion of the second capture element 6002b can be less than the length X4 of the first engagement portion 6047.

In some implementations, the device 6000 can be normally in the capture position (e.g., via a spring, a shape set material, etc.) and actuation element(s) can cause the device 6000 to be in the delivery position during delivery of the device 6000. In some implementations, once the device 6000 is positioned to engage the chordae tendineae CT, the actuation element can release the capture elements 6002a, 6002b to allow the device 6000 to move to the capture position. In some implementations, the device 6000 can be normally in the delivery position (e.g., via a spring, a shape set material, etc.) and actuation element(s) can cause the device 6000 to be moved to the capture position once the device 6000 is positioned to engage the chordae tendineae CT. In some implementations, the device 6000 is not normally in the capture or delivery position but is capable of being moved between the capture and delivery positions via engagement by actuation element(s).

In some implementations, the device 6000 can include one or more locking elements that are configured to maintain the device 6000 in the capture position after engaging the chordae tendineae CT. In the illustrated example, the locking element can be a clip member (e.g., similar to clip member 5966 of device 5900 shown in FIG. 85) that is configured to be secured to the capture elements 6002a, 6002b, where the clip member includes locking extensions 6068 (FIG. 92) that are positioned relative to the capture elements 6002a, 6002b when the clip member is attached to the device 6000 to prevent rotation of the capture elements 6002a, 6002b and, consequently, maintain the capture elements 6002a, 6002b in the capture position. However, it should be understood that any suitable locking element that is capable of maintaining the device 6000 in the capture position can be used. The locking element can be, for example, a clip, a latch, or any other suitable mechanical lock that is capable of maintaining the capture elements 6002a, 6002b in the capture position.

In some implementations, the capture elements 6002a, 6002b are configured to rotate between about 75 degrees and about 105 degrees to move from the delivery position to the capture position, such as between about 80 degrees and about 100 degrees, such as between about 85 degrees and about 95 degrees, such as about 90 degrees. In some implementations, the capture elements 6002a, 6002b can be limited in the amount of rotation that can be achieved. For example, the capture elements 6002a, 6002b can be configured to rotate 115 degrees or less to move between the delivery position and the capture position, such as 110 degrees or less, such as 105 degrees or less, such as 100 degrees or less, such as 95 degrees or less, such as 90 degrees or less. In some implementations, the hub portion (not shown) or any other portion of the device 6000 can include one or more stop members that are configured to prevent further rotation of a corresponding capture element 6002a, 6002b after the capture element 6002a, 6002b achieves the capture position. In some implementations, the capture elements 6002a, 6002b are not limited in the amount of rotation that can be achieved. In some implementations, one of the capture elements 6002a, 6002b can be configured to rotate (e.g., about 180 degrees or less) and the other capture element can be fixed.

Each capture elements 6002a, 6002b can be configured to rotate in a single direction to move from the delivery position to the capture position. For example, the first capture element 6002a can be configured to only rotate in a counterclockwise direction and the second capture element 6002b can be configured to only rotate in clockwise direction when moving from the delivery position to the capture position, or vice versa. In some implementations, the capture elements 6002a, 6002b can be configured to rotate in either the clockwise or counterclockwise direction when moving between the delivery position to the capture position.

Still referring to FIGS. 90-92, the device 6000 is shown being delivered and deployed within the native heart valve (e.g., the tricuspid valve, the mitral valve, etc.) to engage chordae tendineae CT of adjacent leaflets L1, L2. Referring to FIG. 90, the device 6000 can be moved into a ventricle of the heart with the device 6000 is in the delivery position. The capture elements 6002a, 6002b of the device 6000 are positioned substantially perpendicular to a coaptation gap G separating adjacent leaflets L1, L2.

Referring to FIG. 91, the first capture element 6002a can be rotated in a counterclockwise direction CCW such that the first engagement portion 6047 engages a portion of the chordae tendineae CT connected to one leaflet L1 and the second engagement portion 6049 engages a portion of the chordae tendineae CT connected to the adjacent leaflet L2. The second capture element 6002b can be rotated in a clockwise direction CW such that the first engagement portion 6047 engages a portion of the chordae tendineae CT connected to one leaflet L1 and the second engagement portion 6049 engages a portion of the chordae tendineae CT connected to the adjacent leaflet L2. While the first capture element 6002a is shown being rotated in the counterclockwise direction CCW and the second capture element 6002b is shown being rotated in the clockwise direction CW, it should be understood that the first capture element 6002a can rotated in the clockwise direction CW and the second capture element 6002b can be rotated in the counterclockwise direction CCW.

Referring to FIG. 92, the first engagement portion 6047 of the first capture element 6002a and the second engagement portion 6049 of the second capture element 6002b create a first capture area 6053 for securing a portion of the chordae tendineae CT connected to the adjacent leaflets L1, L2, and the second engagement portion 6049 of the first capture element 6002a and the first engagement portion 6047 of the second capture element 6002b create a second capture area 6055 for securing a another portion of the chordae tendineae CT connected to the adjacent leaflets L1, L2. Still referring to FIG. 92, in some implementations, the capture elements 6002a, 6002b can continue rotation in their respective directions until the first engagement portion 6047 of the first capture element 6002a overlaps a portion of the second engagement portion 6049 of the second capture element 6002b, and the second engagement portion 6049 of the first capture element 6002a overlaps a portion of the first engagement portion 6047 of the second capture element 6002b. In some implementations, the capture areas 6053, 6055 are closed by the capture elements 6002a, 6002b to prevent chordae tendineae CT from being released by the device 6000 after implantation.

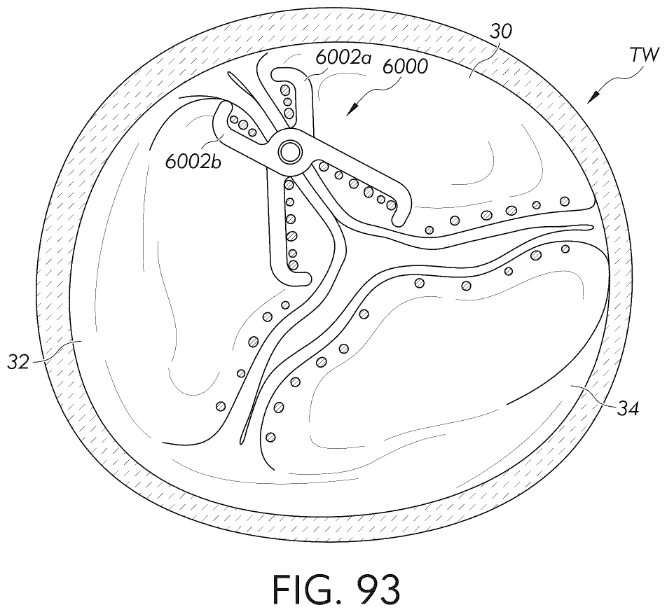

Referring to FIG. 93, the device 6000 is shown with the capture elements 6002a, 6002b in a capture position such that the device 6000 is attached to chordae tendineae CT (e.g., the primary chordae) of each of the anterior leaflet 30 and septal leaflet 32 of the tricuspid valve TV to approximate the leaflets 30, 32 and close a gap therebetween. In some implementations, the device 6000 can be attached to the posterior leaflet 34 and an adjacent leaflet of the tricuspid valve TV in a similar manner. While the tricuspid valve TV is illustrated as being repaired, it should be understood that the device 6000 can be used to repair other valves (e.g., the mitral valve). The device 6000 can be positioned at a variety of locations relative to the leaflets and chordae tendineae of the native heart valve, such as, for example, any of the positions described for device 5600 shown in FIGS. 73-75.

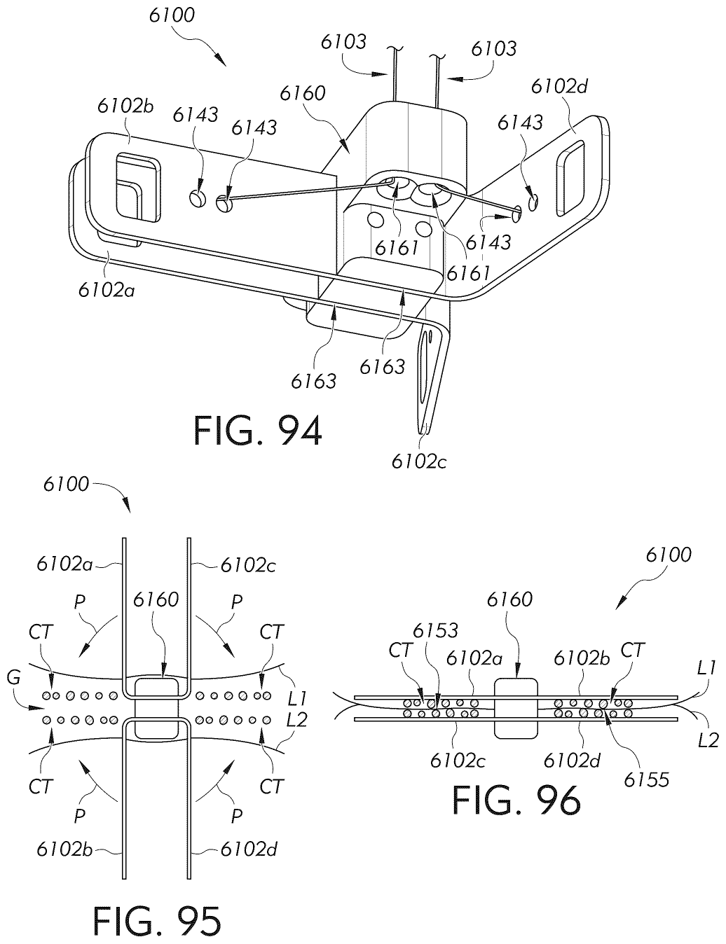

FIGS. 94-97 illustrate a device or implant 6100 (e.g., a prosthetic device, a valve repair device, implantable device, etc.) useable for repairing a native heart valve (e.g., the mitral valve or the tricuspid valve). The device or implant 6100 can include any of the features for another device or implant discussed in the present application or the applications cited above, and the device 6100 can be positioned to engage valve tissue (e.g., leaflets 20, 22, 30, 32, 34) as part of any suitable valve repair system (e.g., any valve repair system disclosed in the present application, or the applications cited above).

The device or implant 6100 can be deployed from a delivery system (e.g., delivery system 102). The delivery system can comprise one or more of a catheter, a sheath, a guide catheter/sheath, a delivery catheter/sheath, a steerable catheter, an implant catheter, a tube, a channel, a pathway, combinations of these, etc. In some implementations, the device or implant 6100 is configured to restore leaflet coaptation, e.g., via chordae approximation, via leaflet approximation, etc.

In some implementations, the device or implant 6100 can include two or more capture elements 6102a-6102d and a base portion 6160 that is attached to the capture elements 6102a-6102d, where each of the capture elements 6102a-6102d are pivotably attached to the base portion 6160. In the illustrated example, the device 6100 includes a first capture element 6102a, a second capture element 6102b, a third capture element 6102c, and a fourth capture element 6102d; where the first and second capture elements 6102a, 6102b are configured to create a first capture area 6153 (FIG. 96) and the third and fourth capture elements 6102c, 6102d are configured to create a second capture area 6155 (FIG. 96).

The capture elements 6102a-6102d can be made of any suitable material, such as, for example, spring materials, such as steel, other metals, plastics, composites, etc. or shape-memory alloys such as Nitinol, or any combination thereof. In some implementations, two or more of the capture elements 6102a-6102d can be integrally formed with each other. In the illustrated example, the first capture element 6102a is integrally formed with the third capture element 6102c, and the second capture element 6102b is integrally formed with the fourth capture element 6102d. In this example, the base portion 6160 includes slots 6163 for receiving a central portion of the integrally formed first and third capture elements 6102a, 6102c and the integrally formed second and fourth capture elements 6102b, 6102d.

In some implementations, the capture element 6102a, 6102b can be configured to engage multiple chordae tendineae CT to distribute the tension force between multiple chordae to protect the chordae from being overloaded. The capture elements 6102a, 6102b can be configured in a variety of ways, including different cross-sectional shapes, different sizes (e.g., length, width, thickness), composed of different materials, etc.

One or more actuation elements 6103 can be configured to engage the capture elements 6102a-6102d to move the capture elements between a delivery position (as shown in FIG. 95) and a capture position (as shown in FIG. 96). The actuation elements 6103 can be, for example, rods, shafts, tubes wires, sutures, or any other suitable components that are capable of pivoting the capture elements 6102a-6102d relative to the base portion 6160. The base portion 6160 can include one or more openings 6161 for receiving the actuation elements 6103, and the capture elements 6102a-6102c can include one or more openings 6143 for receiving the actuation elements 6103.

The device 6100 can be configured such that pivoting the first and second capture elements 6102a, 6102b towards each other creates the first capture area 6153 (FIG. 96) and pivoting the third and fourth capture elements 6102c, 6102d towards each other creates the second capture area 6155 (FIG. 96). In some implementations, the device 6100 can be normally in the capture position (e.g., via a spring, a shape set material, etc.) and actuation elements 6103 can cause the device 6100 to be in the delivery position during delivery of the device 6100. In some implementations, once the device 6100 is positioned to engage the chordae tendineae CT, the actuation elements 6103 can release the capture elements 6102a-6102d to allow the device 6100 to move to the capture position. In some implementations, the capture elements 6102a-6102d can be moved between the delivery and capture positions via a mechanical device (e.g., a gear mechanism).

In the illustrated example, each capture element 6102a-6102d has a separate actuation element 6103 associated therewith, which allows for each of the capture elements 6102a-6102d to be moved between the delivery and capture positions independently of each other. For example, referring to FIG. 94, the first and second capture elements 6102a, 6102b are shown in the capture position while the third and fourth capture elements 6102c, 6102d are shown in the delivery position. This allows a user to engage and/or capture a portion of the chordae tendineae of adjacent leaflets with the first and second capture elements 6102a, 6102b to attach the device 6100 thereto, and then attempt to engage and/or capture another portion of the chordae tendineae of adjacent leaflets with the third and fourth capture elements 6102c, 6102d after the device 6100 is connected to the chordae tendineae, which can make it easier to secure the device in a desired position relative to the chordae tendineae and the leaflets. In some implementations, the device 6100 is not normally in the capture or delivery position but is capable of being moved between the capture and delivery positions via engagement by actuation element(s).

In some implementations, the device 6100 can include one or more locking elements (not shown) that are configured to maintain the device 6100 in the capture position after engaging the chordae tendineae CT. Any suitable locking element that is capable of holding the device 6100 in the capture position can be used. The locking element can be, for example, a clip, a latch, or any other suitable mechanical lock that is capable of maintaining the capture elements 6102a, 6102b in the capture position.

Referring to FIGS. 95-96, the device 6100 is shown being delivered and deployed within the native heart valve (e.g., the tricuspid valve, the mitral valve, etc.) to engage chordae tendineae CT of adjacent leaflets L1, L2. Referring to FIG. 95, the device 6100 can be moved into a ventricle of the heart with the device 6100 is in the delivery position. The capture elements 6102a-6102d are positioned substantially perpendicular to a coaptation gap G separating adjacent leaflets L1, L2.

Referring to FIG. 95, the capture elements 6102a-6102d can be pivoted in a direction P (independently or simultaneously) such that the capture elements 6102a-6102d are in the capture position (as shown in FIG. 96). The pivoting of the capture elements 6102a, 6102b causes the first capture element 6102a to engage a portion of the chordae tendineae CT of one leaflet L1 and the second capture element 6102b to engage a portion of the chordae tendineae CT of an adjacent leaflet L2 such that these portions of the chordae tendineae CT of the adjacent leaflets L1, L2 are secured in the first capture area 6153. The pivoting of the capture elements 6102c, 6102d causes the third capture element 6102c to engage another portion of the chordae tendineae CT of the leaflet L1 and the fourth capture element 6102d to engage another portion of the chordae tendineae CT of the adjacent leaflet L2 such that these portions of the chordae tendineae CT of the adjacent leaflets L1, L2 are secured in the second capture area 6155.

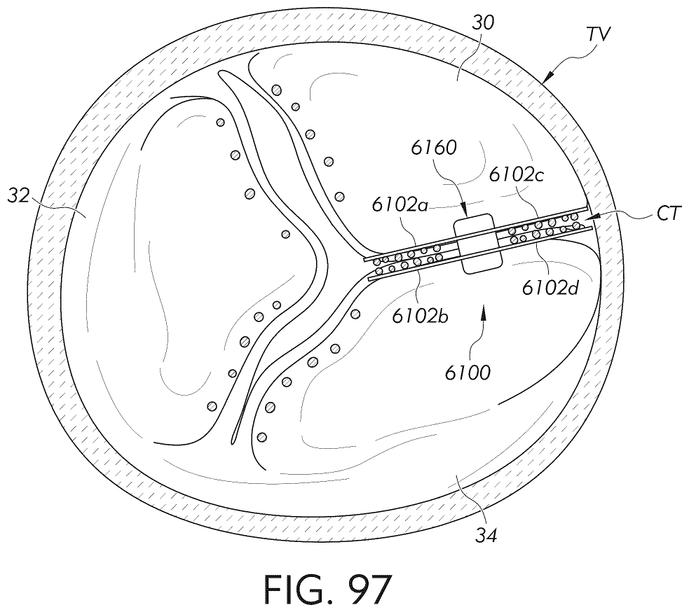

Referring to FIG. 97, the device 6100 is shown with the capture elements 6102a-6102d in a capture position such that the device 6100 is attached to chordae tendineae CT (e.g., the primary chordae) of each of the anterior leaflet 30 and posterior leaflet 34 of the tricuspid valve TV to approximate the leaflets 30, 34 and close a gap therebetween. In some implementations, the device 6100 can be attached to the septal leaflet 32 and an adjacent leaflet of the tricuspid valve TV in a similar manner. While the tricuspid valve TV is illustrated as being repaired, it should be understood that the device 6100 can be used to repair other valves (e.g., the mitral valve). The device 6100 can be positioned at a variety of locations relative to the leaflets and chordae tendineae of the native heart valve, such as, for example, any of the positions described for device 5600 shown in FIGS. 73-75. While many examples herein involve capturing chordae tendineae, the principles and features can be similarly used to capture other types of tissue.

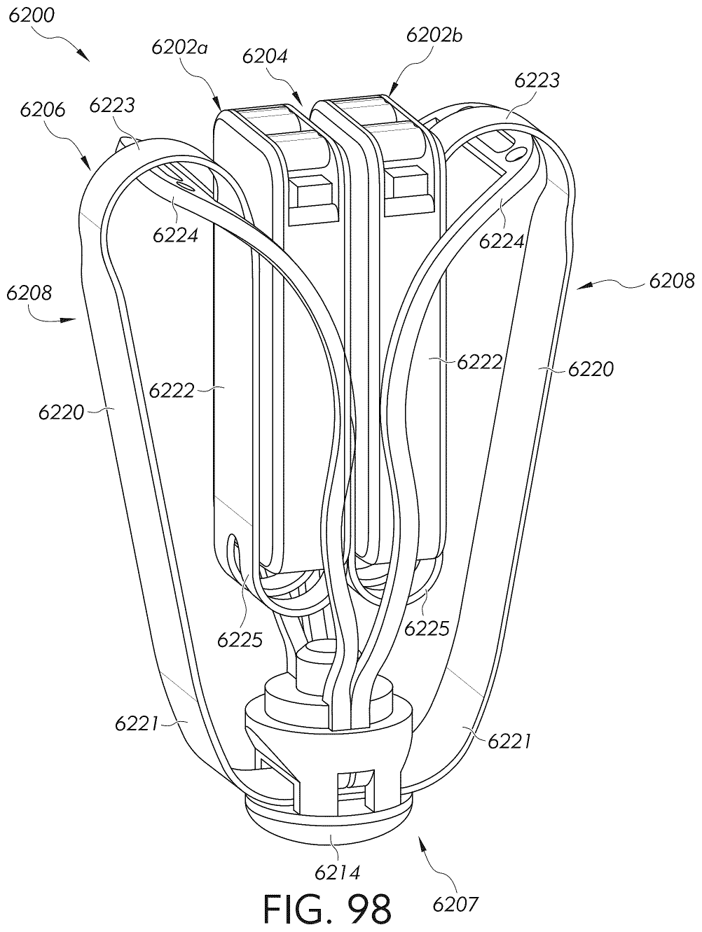

FIGS. 98-110 illustrate a device or implant 6200 (e.g., a prosthetic device, a valve repair device, implantable device, etc.) useable for repairing a native heart valve (e.g., the mitral valve or the tricuspid valve). The device or implant 6200 can include any of the features for another device or implant discussed in the present application or the applications cited above, and the device 6200 can be positioned to engage valve tissue (e.g., leaflets 20, 22, 30, 32, 34) as part of any suitable valve repair system (e.g., any valve repair system disclosed in the present application, or the applications cited above).

The device or implant 6200 can be deployed from a delivery system (e.g., delivery system 102). The delivery system can comprise one or more of a catheter, a sheath, a guide catheter/sheath, a delivery catheter/sheath, a steerable catheter, an implant catheter, a tube, a channel, a pathway, combinations of these, etc. In some implementations, the device or implant 6200 is configured to restore leaflet coaptation, e.g., via chordae approximation, via leaflet approximation, etc.

In some implementations, the device 6200 includes a capture portion 6204, an anchor portion 6206, and a distal portion 6207. The capture portion 6204 of the device can include one or more capture elements 6202a, 6202b for engaging chordae tendineae CT of adjacent leaflets of a native valve. In some implementations, the anchor portion 6206 includes a plurality of anchors 6208. The anchors 6208 can be configured in a variety of ways. In some implementations, each anchor 6208 includes outer paddles 6220, inner paddles 6222, and paddle extension members or paddle frames 6224.

In some implementations, the paddles 6220, 6222 are formed from a flexible material that can be a metal fabric, such as a mesh, woven, braided, or formed in any other suitable way or a laser cut or otherwise cut flexible material. The material can be cloth, shape-memory alloy wire or sheet—such as Nitinol—to provide shape-setting capability, or any other flexible material suitable for implantation in the human body.

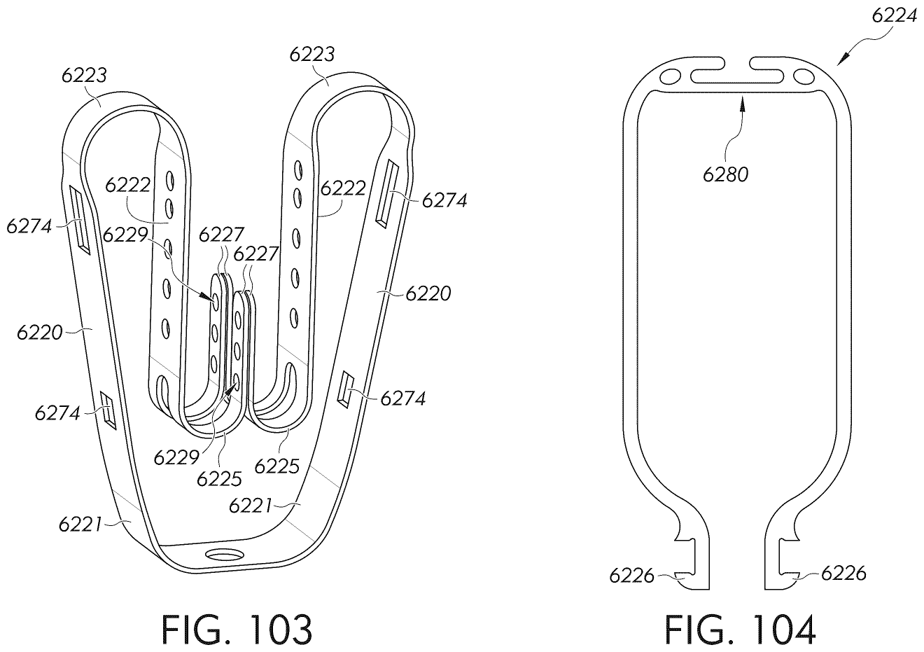

In some implementations, the outer paddles 6220 are jointably attached to the cap 6214 of the distal portion 6207 by connection portions 6221 and to the inner paddles 6222 by connection portions 6223. The inner paddles 6222 are jointably attached to each other by connection portions 6225 and connection portions 6227 (FIG. 103). In some examples, connection portions 6227 can include one or more openings 6229 (FIG. 103) for receiving a fastener to attach the inner paddles 6222 together. In this manner, the anchors 6208 are configured similar to legs in that the inner paddles 6222 are like upper portions of the legs, the outer paddles 220 are like lower portions of the legs, and the connection portions 6223 are like knee portions of the legs. In some implementations, the inner paddles 6222 are stiff, relatively stiff, rigid, have rigid portions and/or are stiffened by an attachment with a corresponding capture elements 6202a, 6202b.

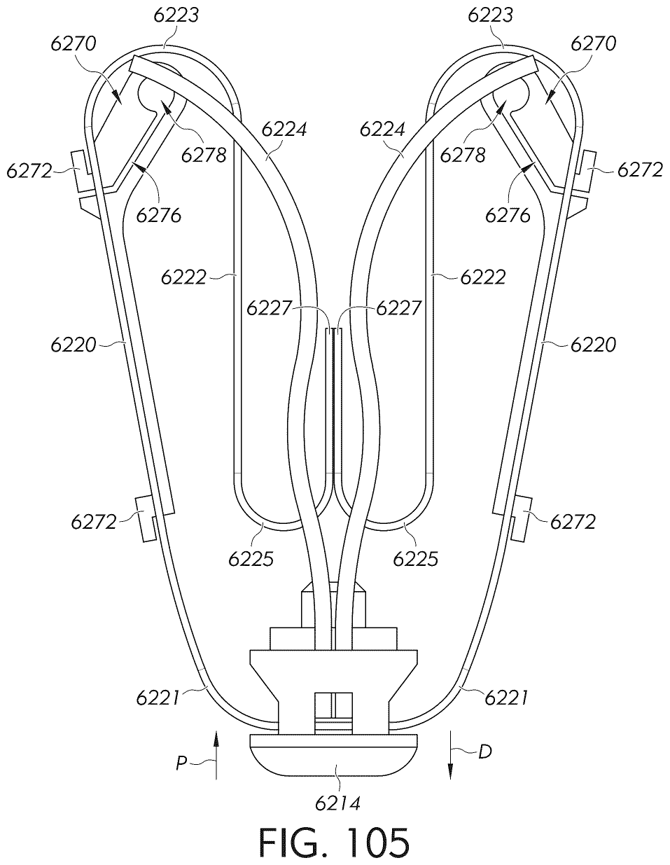

In some implementations, the paddle frames 6224 can have connection portions 6226 that are attached to the cap 6214 of the distal portion 6207, and the paddle frames 6224 can extend to the connection portions 6223 between the inner and outer paddles 6222, 6220. The paddle frames 6224 can be attached to the outer paddles 6220 by connection elements 6270 (FIG. 105). For example, the connection elements 6270 can have one or more protrusions 6272 (FIG. 105) for extending through openings 6274 (FIG. 103) of the outer paddle 6220 to attach the connection elements 6270 to the outer paddles 6220, and the connection elements 6270 can have a slot 6276 (FIG. 105) extending to an opening 6278 (FIG. 105) for receiving a connection portion 6280 (FIG. 104) of the paddle frame 6224. In some implementations, the paddle frames 6224 are formed of a material that is more rigid and stiff than the material forming the paddles 6222, 6220 so that the paddle frames 6224 provide support for the paddles 6222, 6220. The paddle frames 6224 can take any suitable form, such as, for example, the form of the paddle frames 224 shown in FIG. 22, the form of the paddle frames 324 shown in FIG. 23, or any other form for a paddle frame described in the present application.

The connections between the paddle frames 6224, the outer and inner paddles 6220, 6222, the cap 6214, and the capture elements 6202a, 6202b can constrain each of these parts to the movements and positions described herein. In particular, the connection portion 6223 is constrained by its connection between the outer and inner paddles 6220, 6222 and by its connection to the paddle frame 6224. Similarly, the paddle frame 6224 is constrained by its attachment to the connection portion 6223 (and thus the inner and outer paddles 6222, 6220) and to the cap 6214.

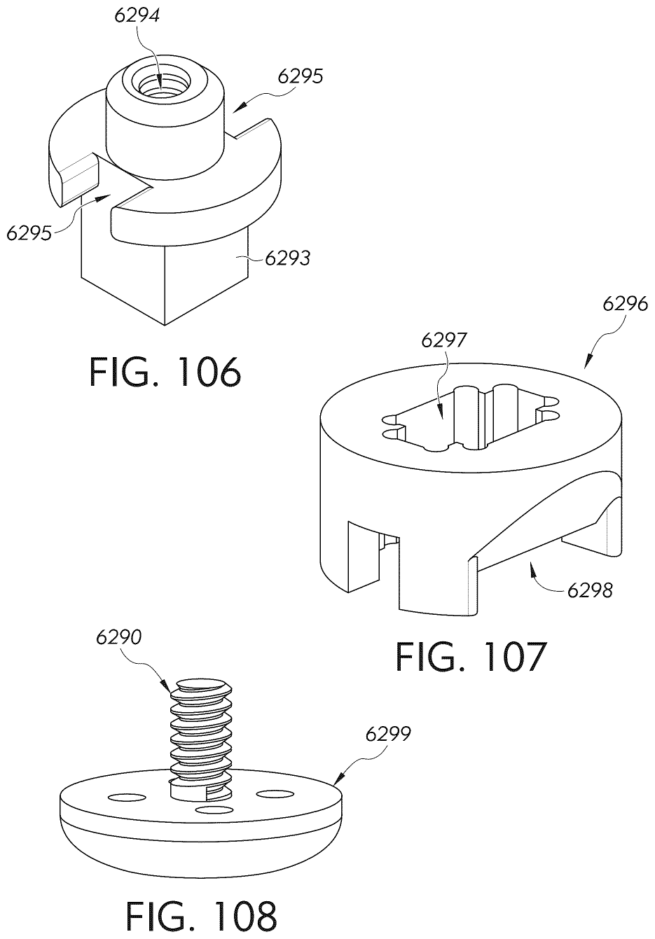

In some implementations, the distal portion 6207 includes a cap 6214 configured to attach to the anchors 6208 and receive an actuation element (e.g., actuation wire, shaft, tube, hypotube, line, suture, braid, etc.) of the delivery system. The actuation element can be configured to removably engage the cap 6214 with a threaded connection, or the like, so that the actuation element can be disengaged and removed from the device 6200 after implantation. Referring to FIG. 106, the cap 6214 can include a first portion 6293 having an opening 6294 for receiving an actuation element and openings or slots 6295 for receiving connection portions 6226 of frame 6224. Referring to FIG. 107, the cap 6214 can include a second portion 6296 having an opening 6297 for receiving the first portion 6293 of the cap 6214 and the connection portions 6226 of the paddle frame 6224. The second portion 6296 can include one or more openings 6298 for receiving the connection portions 6221 of the outer paddles 6220. Referring to FIG. 108, the cap 6214 can include a third portion 6299 that is configured to extend through the second portion 6296 and attach to the first portion 6293 to secure the portions 6293, 6296, 6299 together. For example, the third portion 6299 can include a connection member 6290 (e.g., a threaded connector) that is configured to attach to the first portion 6293. The third portion 6299 can define a distal portion of the opening 6298 of the second portion 6296 to secure the outer paddles 6220 (FIGS. 98 and 105) to the cap 6214.

Referring to FIGS. 98-110, in some implementations, the capture element(s) 6202a, 6202b are configured to move between a delivery configuration (as shown in FIGS. 98-100) and a capture configuration (as shown in FIG. 102), where the capture elements 6202a, 6202b are configured to engage tissue, such as leaflets and/or chordae tendineae CT (e.g., the primary chordae (closest to the edge of the leaflet)) of two or more leaflets to pull the leaflets together when in the expanded configuration; thus, reducing the gap between the leaflets.

In some implementations, the paddle(s) 6208 of the device 6200 are moveable between an open configuration (not shown) and a closed configuration (as shown in FIGS. 98 and 105), where the movement of the paddle(s) 6208 from the open configuration to the closed configuration allows for the capture elements 6202a, 6202b to engage the tissue (e.g., engage chordae tendineae CT to pull the leaflets together, etc.). For example, movement of the cap 6214 in a distal direction D (FIG. 105) with the actuation element can cause the paddles 6208 to move the open position, and movement of the cap 6214 in the proximal direction P (FIG. 105) can cause the paddles 6208 to move to the closed position.

In some implementations, the capture element 5602 can be configured to engage multiple chordae tendineae CT to distribute the tension force between multiple chordae to protect the chordae from being overloaded.

Referring to FIGS. 99-102, each of the capture elements 6202a, 6202b can include a housing 6271 and an engagement element 6273. The housing 6271 can include a channel 6275 for receiving and allowing movement of the engagement element 6273 within the housing 6271, a first opening 6277, and a second opening 6279. The housing can be made of any suitable material, such as, for example, plastics, metals, etc.

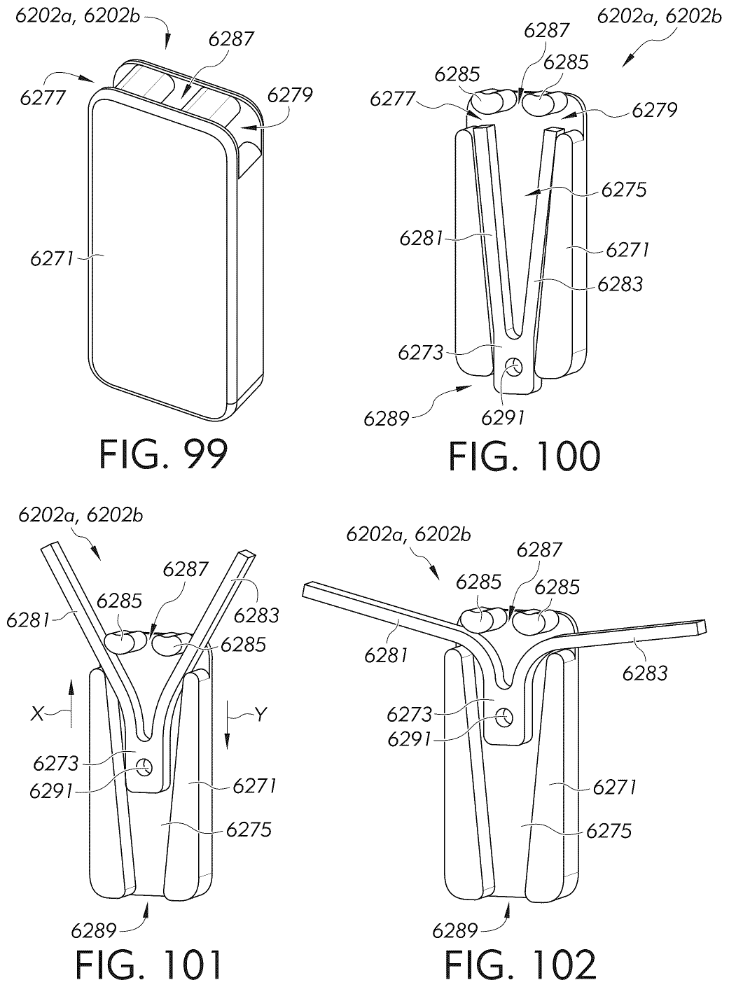

In some implementations, the engagement element 6273 can include a first engagement member 6281 and a second engagement member 6283. The engagement element 6273 can be movable between a compressed configuration (as shown in FIG. 100) when the engagement members 6281, 6283 are disposed within the housing 6271 and an expanded configuration (as shown in FIG. 102) when the engagement members 6281, 6283 extend through the openings 6277, 6279 of the housing 6271. The engagement element 6273 can be made of any suitable material that allows for movement between the compressed and expanded configurations, such as, for example, spring materials, such as steel, other metals, plastics, composites, etc. or shape-memory alloys such as Nitinol, or any combination thereof. In some implementations, the engagement members 6281, 6283 are integrally formed each other. In some implementations, the engagement elements 6273 can be normally in the expanded configuration (e.g., via a shape set material), where containment of the engagement elements within the corresponding housing 6271 causes the engagement elements 6273 to be in a compressed configuration. The engagement elements 6273 can be configured in a variety of ways, including different cross-sectional shapes, different sizes (e.g., length, width, thickness), composed of different materials, etc.

In some implementations, the housing 6271 can be configured to facilitate movement of the engagement element 6273 between the compressed and expanded configurations. For example, the channel 6275 can be chamfered or sloped to facilitate movement of the engagement element 6273 to the expanded configuration when moved in a proximal direction X (FIG. 101), and to facilitate movement of the engagement element 6273 to the compressed configuration when moved in the distal direction Y (FIG. 101). In some implementations, the housing 6271 can include one or more proximal members 6285 that are positioned to engage the engagement members 6281, 6283 proximate the corresponding openings 6277, 6279. The proximal members 6285 can be shaped to facilitate movement of the engagement members 6281, 6283 through the corresponding opening 6277, 6279. For example, the proximal members 6285 can have a curved or arcuate shape, or any other suitable shape that facilitates movement of the engagement members 6281, 6283 in an outward direction such that the engagement element 6273 can be moved to the expanded configuration.

One or more actuation elements (e.g., actuation wire, shaft, tube, hypotube, line, suture, braid, etc.) of the delivery system can be configured to engage the engagement elements 6273 to move the engagement elements 6273 between the compressed and expanded configurations. In some implementations, the actuation element can extend through a proximal opening 6287 of the housing 6271 to engage the engagement element 6273. In some implementations, the actuation element can extend through a distal opening 6289 of the housing 6271 to engage the engagement element 6273. In some implementations, actuation elements can extend through the openings 6277, 6279 to engage the engagement element 6273. The engagement element 6273 can have an opening 6291 for attaching to an actuation element.

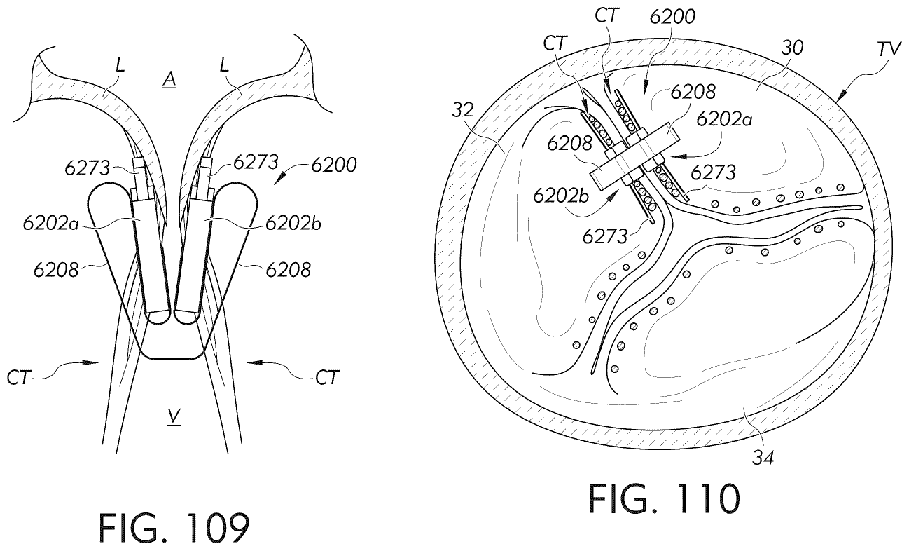

Referring to FIG. 109, the device 6200 can be delivered and deployed within a native heart valve (e.g., the tricuspid valve, the mitral valve, etc.) in a similar manner to the device 5600 shown in FIGS. 68-71. For example, a delivery sheath/catheter (not shown) can be inserted into the atrium A of the heart and the implant/device 6200 and an actuation element (e.g., an actuation catheter or tube, a wire, a suture etc.) can be deployed from the delivery catheter/sheath. The device 6200 can be moved through the native heart valve and into the ventricle with the paddles 6208 in the open position and the capture elements 6202a, 6202b in the compressed configuration (as shown in FIGS. 98-100). The capture elements 6202a, 6202b being in the compressed configuration allow for the paddles 6208 and capture elements 6202a, 6202b to extend through and behind the chordae tendineae CT without pushing the chordae tendineae CT outward.

In some implementations, once the capture elements 6202a. 6202b are positioned behind the chordae tendineae, the capture elements 6202a. 6202b can be moved to the expanded configuration (as shown in FIG. 102) such that the engagement elements 6273 of the capture elements 6202a, 6202b extend behind and across one or more chordae. After the capture elements 6202a. 6202b are positioned behind and extend across one or more chordae tendineae, the paddles 6208 can be moved to the closed position such that the capture element 6202a. 6202b engage the chordae tendineae of adjacent leaflets to approximate the chordae and, consequently, approximate the adjacent leaflets L. The device 6200 can be positioned at a variety of locations relative to the leaflets L and chordae tendineae CT of the native heart valve, such as, for example, any of the positions described for device 5600 shown in FIGS. 73-75. While many examples herein involve capturing chordae tendineae, the principles and features can be similarly used to capture other types of tissue.

Referring to FIG. 110, the device 6200 is shown with the capture elements 6202a, 6202b in a capture position such that the device 6200 is attached to chordae tendineae CT (e.g., the primary chordae) of each of the anterior leaflet 30 and septal leaflet 32 of the tricuspid valve TV to approximate the leaflets 30, 32 and close a gap therebetween. In some implementations, the device 6200 can be attached to the posterior leaflet 34 and an adjacent leaflet of the tricuspid valve TV in a similar manner. While the tricuspid valve TV is illustrated as being repaired, it should be understood that the device 6200 can be used to repair other valves (e.g., the mitral valve).

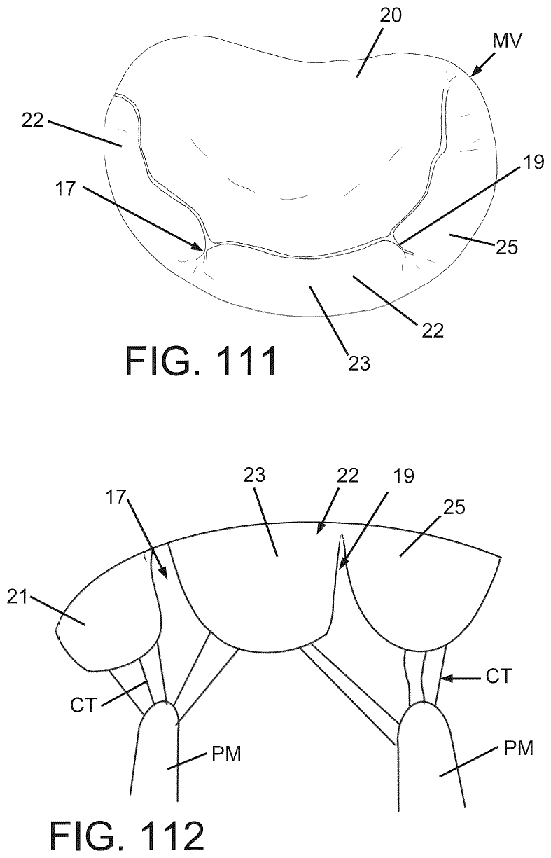

Referring to FIGS. 111 and 112, the mitral valve MV includes two leaflets, the anterior leaflet 20 and the posterior leaflet 22. The mitral valve MV also includes an annulus (e.g., the annulus 24 shown in FIG. 5), which is a variably dense fibrous ring of tissues that encircles the leaflets 20, 22. The posterior leaflet 22 has a first cleft 17 and a second cleft 19 that separate the posterior leaflet 22 into a first portion 21, a second portion 23, and a third portion 25. Referring to FIG. 112, the mitral valve MV is anchored to the wall of the left ventricle LV by chordae tendineae CT and papillary muscles PM. The mitral valve MV opens and closes in response to pressure changes in the left atrium LA and the left ventricle LV.

Mitral regurgitation can occur when the mitral valve fails to close properly (e.g., when the anterior and posterior leaflets 20, 22 do not coapt) and blood flows into the left atrium from the left ventricle during the systolic phase of heart contraction. Various devices or implants (including various devices or implants described in the present application) are configured to directly or indirectly (e.g., via the chordae tendineae CT) engage adjacent leaflets (e.g., adjacent leaflets of the mitral valve or the tricuspid valve) to cause the leaflets to coapt and prevent regurgitation. However, such devices or implants may not prevent regurgitation of blood through the mitral valve via the clefts 17, 19 under some circumstances. The size of the clefts 17, 19 can vary depending on the person and/or if the clefts 17, 19 have become larger (e.g., stretched out) over time. In certain instances, the size of one or both of the clefts 17, 19 can be large enough to allow for regurgitation through the mitral valve MV during the systolic phase of heart contraction.

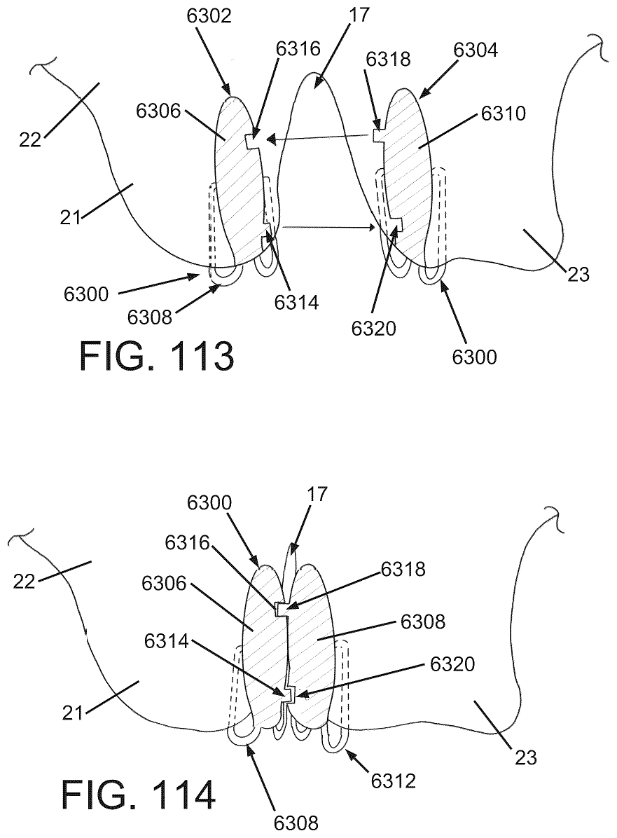

FIGS. 113-119 illustrate various devices that are configured to treat, prevent, and/or reduce regurgitation through native valve, e.g., through the clefts 17, 19 of the mitral valve MV, through clefts in other valves or leaflets of other valves, between different leaflets, at commissures of a valve, etc.

FIGS. 113-114 illustrate a device or implant 6300 (e.g., a prosthetic device, a valve repair device, implantable device, etc.) useable for repairing a native heart valve (e.g., the mitral valve or the tricuspid valve). The device or implant 6300 can include any of the features for another device or implant discussed in the present application or the applications cited above, and the device 6300 can be positioned to engage valve tissue (e.g., leaflets 20, 22, 30, 32, 34) as part of any suitable valve repair system (e.g., any valve repair system disclosed in the present application, or the applications cited above).

In some implementations, the device or implant 6300 can be deployed from a delivery system (e.g., delivery system 102). The delivery system can comprise one or more of a catheter, a sheath, a guide catheter/sheath, a delivery catheter/sheath, a steerable catheter, an implant catheter, a tube, a channel, a pathway, combinations of these, etc.

In some implementations, the device or implant 6300 is configured to restore leaflet coaptation by reducing a region between tissues or portions of tissue. In some implementations this is done by reducing the size of one or more of clefts in a valve or leaflet. In some implementations, this is done by reducing a gap between leaflets or portions of a leaflet.

The device or implant 6300 can be delivered via a transeptal approach (in which the device 6300 is inserted into the left atrium via the inferior or superior vena cava), or the device 6300 can be delivered via a transapical approach (in which the device 6300 is inserted into the left ventricle via the apex of the heart). It should be understood, however, that the device 6300 can be delivered in any suitable manner that allows for the device 6300 to engage the mitral valve MV or other native valve.

In some implementations, the device 6300 can include a first capture element configured as a first clip member 6302 and a second capture element configured as a second clip member 6304, where the first and second clip members are configured to be attached to the adjacent portions 21, 23 of the native valve (e.g., of a mitral valve MV, etc.) and then attached to each other to close a region or gap, such as cleft 17.

In some implementations, the first clip member 6302 can include a clip body 6306 and an attachment portion 6308, where the first clip member 6302 is configured to be moved between an open position (in which there is an opening between the clip body 6306 and the attachment portion 6308) and a closed position (in which the attachment portion 6308 engages or is proximate the clip body 6306 such that the first clip member can be secured to the posterior leaflet 22 or any other portion of a native heart valve).

In some implementations, the second clip member 6304 can include a clip body 6310 and an attachment portion 6312, where the second clip member 6304 is configured to be moved between an open position (in which there is an opening between the clip body 6310 and the attachment portion 6312) and a closed position (in which the attachment portion 6312 engages or is proximate the clip body 6310 such that the second clip member can be secured to the posterior leaflet 22 or any other portion of a native heart valve).

The first and second clip members 6302, 6304 can be moved between the open and closed positions by any suitable means, such as, for example, any means described in the present application. In some implementations, the first and second clip members 6302, 6304 can be shape set in the closed position and an actuation element (e.g., an actuation wire, suture, etc.) can be used to move the first clip member to the open position during implantation of the first and second clip members 6302, 6304.

In some implementations, the first and second clip members 6302, 6304 are configured to be moved between a disengaged position (as shown in FIG. 113) and an engaged position (as shown in FIG. 114). For example, the first and second clip members 6302, 6304 can be attached to the respective adjacent leaflet portions 21, 23 such that the first and second clip members 6302, 6304 are in the engaged position and then the first and second clip members 6302, 6304 can be moved to the engaged position to close the cleft 17 between the first and second leaflet portions 21, 23. In some implementations, one or more actuation elements (e.g., actuation wires, an actuation tubes, sutures, etc.) can be used to engage the clip members 6302, 6304 to move the clip members 6302, 6304 between the disengaged and engaged positions.

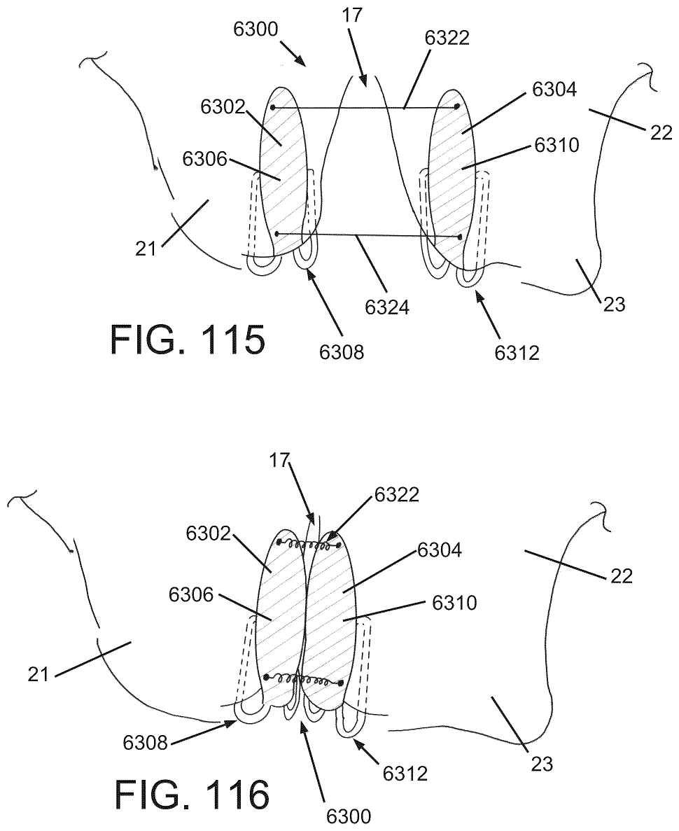

Referring to FIGS. 115-116, in some implementations, the first and second clip members 6302, 6304 can be attached by a first resilient member 6322 and a second resilient member 6324, where the first and second resilient members 6322, 6324 are configured to move the first and second clip members 6302, 6304 from the disengaged position to the engaged position after the clip members 6302, 6304 are implanted on adjacent leaflet portions 21, 23 of the posterior leaflet 22.

In some implementations, the first and second resilient members 6322, 6324 can include one or more biasing members (e.g., springs, shape set material (e.g., Nitinol), etc.) that cause the clip members 6302, 6304 to be in a normally engaged position (as shown in FIG. 116). In some implementations, one or more actuation elements (e.g., actuation wires, an actuation tubes, sutures, etc.) can be used to maintain the clip members 6302, 6304 in the disengaged position (as shown in FIG. 115) when the clip members 6302, 6304 are implanted on the leaflet portions 21, 23, and removal of the force provided by the actuation element(s) allows for the clip members 6302, 6304 to move to the normally engaged position to close the cleft 17 between the leaflets.

In some implementations, the first and second resilient members 6322, 6324 can include NiTi wire that is in an extended position during implantation of the clip members 6302, 6304 (in which the clip members 6302, 6304 are in the disengaged position), and the NiTi wire can be heated (e.g., via current applied through a delivery catheter or any other means for applying heat to the NiTi wire) to cause the NiTi wire to move to a compressed position and cause the clip members 6302, 6304 to move to the engaged position. While the illustrated example shows the first and second clip members 6302, 6304 attached by two resilient members 6322, 6324, it should be understood that the device or implant 6300 can include any suitable number of resilient members that are configured to move the first and second clip members 6302, 6304 from the disengaged position (as shown in FIG. 115) to the engaged position (as shown in FIG. 116).

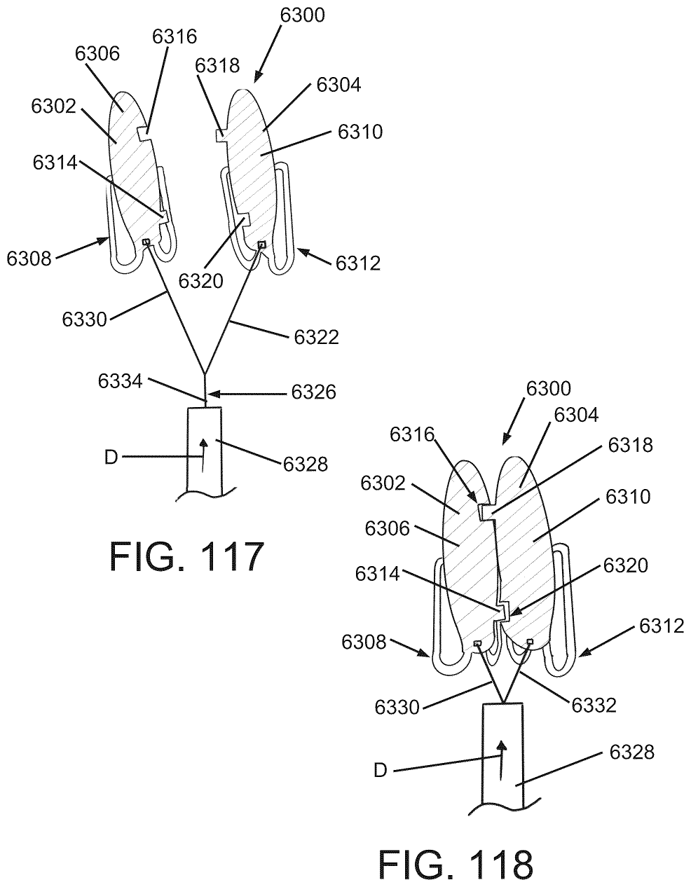

Referring to FIGS. 117-118, in some implementations, one or more actuation elements engage the clip members 6302, 6304 to move the clip members 6302, 6304 between the disengaged and engaged positions. The one or more actuation elements can include a bifurcated member 6326 (e.g., a wire, a suture, etc.) and a sheath or tube 6328. The bifurcated member 6326 includes a proximal portion 6334 that divides into a first distal portion 6330 and a second distal portion 6332. In some implementations, the first distal portion 6330 of the bifurcated member 6326 is attached to the first clip member 6302, and the second distal portion 6332 of the bifurcated member 6326 is attached to the second clip member 6304. FIG. 117 shows the clip members 6302, 6304 being in the disengaged position.

In some implementations, movement of the tube 6328 in a distal direction D causes the tube 6328 to engage the first and second distal portions 6330, 6332 of the bifurcated member 6326, which causes the first and second distal portions 6330, 6332 and, consequently, the first and second clip members 6302, 6304 to move towards each other and into the engaged position (as shown in FIG. 118).

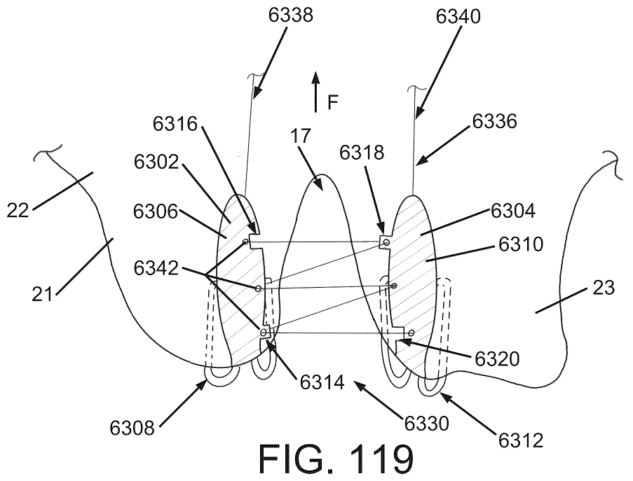

Referring to FIG. 119, in some implementations, one or more actuation elements engage the clip members 6302, 6304 to move the clip members 6302, 6304 between the disengaged and engaged positions. The actuation elements can include a wire or suture 6336 that is threaded through each of the first and second clip members 6302, 6304. In the illustrated example, the first clip member 6302 includes openings 6342 for receiving the wire or suture 6336 and the second clip member includes openings 6344 for receiving the wire or suture 6336. While the first clip member 6302 is shown as having three openings 6342 for receiving the wire or suture 6336, it should be understood that the first clip member can have any suitable number of openings 6342. While the second clip member 6304 is shown as having three openings 6344 for receiving the wire or suture 6336, it should be understood that the second clip member can have any suitable number of openings 6344.

In some implementations, the wire or suture 6336 has first and second ends 6338, 6340 that extend through a delivery system such that a user can engage one or both of the first and second ends 6338, 6340 to apply a force F to the wire or suture 6336 to cause the first and second clip members 6302, 6304 to move from the disengaged position (as shown in FIG. 119) the engaged position (e.g., similar to the position shown in FIG. 114).

Referring to FIGS. 113-119, the first capture element or first clip member 6302 can include one or more locking features, and the second capture element or second clip member 6304 can include one or more locking features that are configured to engage the locking features of the first clip member 6302. In the illustrated example, the clip body 6306 of the first clip member 6302 can include a first locking protrusion 6314 and a first locking receptacle 6316, and the clip body 6310 of the second clip member 6304 can include a second locking protrusion 6318 and a second locking receptacle 6318.

In some implementations, the first locking receptacle 6316 of the first clip member 6302 is configured to receive the second locking protrusion 6318 of the second clip member 6304, and the second locking receptacle 6320 of the second clip member 6304 is configured to receive the first locking protrusion 6314 of the second clip member 6302, where the engagement between these locking features secure the first and second clip members 6302, 6304 in the engaged position. While the illustrated example shows the clip members 6302, 6304 having respective locking protrusions 6314, 6318 and locking receptacles 6316, 6320, it should be understood that the clip members 6302, 6304 can include any suitable type of locking features that are capable of securing the clip members 6302, 6304 together in the engaged position.

In the illustrate example, the device 6300 is shown being attached to a first portion 21 and a second portion 23 of the posterior leaflet 22 to close the cleft 17 that is disposed between these portions 21, 23. However, it should be understood that the device 6300 can be secured to the second and third portions 23, 25 (FIGS. 111-112) of the posterior leaflet 22 to close the cleft 19 (FIGS. 111-112), or the device 6300 can be attached to any other suitable portion of a native heart valve that will allow for the device 6300 to reduce a region of the heart valve (e.g., reduce a cleft, gap, or opening of the native heart valve between portions of the same leaflet, between two different leaflets, in a commissural area, etc.). Further, the device can be adapted for use on other anatomy and tissue types outside of the heart.

### EXAMPLES

Example 1. A device useable for repairing/treating a native valve, comprising: (a) a leaflet coaptation assist element configured to be positioned between a first leaflet and a second leaflet of the native valve to inhibit the first leaflet and the second leaflet from prolapsing; (b) a first anchor configured to engage a first ventricular side of the first leaflet; (c) a first line connected to the first anchor and extending through the first leaflet to a first atrial side of the first leaflet; (d) a second anchor configured to engage a second ventricular side of the second leaflet; and/or (e) a second line connected to the second anchor and extending through the second leaflet to a second atrial side of the second leaflet, wherein the first line, the first anchor, the second line, and the second anchor are configured to pull the first leaflet and the second leaflet into a repair position.

Example 2. The device of example 1, wherein the leaflet coaptation assist element includes a first arm connected to a second arm by a joint portion.

Example 3. The device of example 2, wherein the first arm has a first outer surface for engaging the first leaflet and the second arm has a second outer surface of engaging the second leaflet.

Example 4. The device of any one of examples 2-3, wherein the leaflet coaptation assist element is configured for the first line to extend through the first arm and the second line to extend through the second arm.

Example 5. The device of any one of examples 1-5, wherein the leaflet coaptation assist element has a delivery state for deploying through a catheter and a deployed state for positioning in the native valve between the first leaflet and the second leaflet.

Example 6. The device of any one of examples 1-5, further comprising a lock configured to engage the first line and the second line to secure the first leaflet and the second leaflet in the repair position; and/or optionally wherein the leaflet coaptation assist element is configured to be removed after the lock has locked the first leaflet and the second leaflet.

Example 7. The device of example 6, further comprising a first spacer configured to be positioned between the lock and the first leaflet and a second spacer configured to be positioned between the lock and the second leaflet.

Example 8. The device of example 7, wherein the first spacer is configured to receive the first line therethrough and the second spacer is configured to receive the second line therethrough.

Example 9. The device of any one of examples 6-8, further comprising a first retrieval line attached to a first arm of the leaflet coaptation assist element and a second retrieval line attached to a second arm the leaflet coaptation assist element to withdraw the leaflet coaptation assist element from between the first leaflet and the second leaflet after the lock is in place.

Example 10. The device of any one of examples 1-8, wherein the leaflet coaptation assist element includes a body having a compressible portion configured to be longitudinally compressed into a deployed state.

Example 11. The device of example 10, wherein the body includes a braided material.

Example 12. The device of any one of examples 10-11, wherein the compressible portion defines a spinning top shape when in the deployed state.

Example 13. The device of any one of examples 10-12, wherein the compressible portion includes a distal end attached to an actuation element extending through the compressible portion and a proximal end axially movable relative to the distal end and the actuation element.

Example 14. The device of example 13, wherein the proximal end includes a stop configured to engage a positioning element for moving the compressible portion from the delivery state to the deployed state.

Example 15. The device of any one of examples 1-14, further comprising a retaining member configured to engage the first leaflet to retain the first leaflet in position to be engage by the first anchor.

Example 16. The device of example 15, wherein the retaining member includes a stem configured to extend distally from the leaflet coaptation assist element and an arm extending proximally from a distal end of the stem at an acute angle.

Example 17. The device of example 16, wherein the arm is configured to extend to a ventricular side of the first leaflet to position the first leaflet between the arm and the leaflet coaptation assist element.

Example 18. The device of any one of examples 15-17, further comprising a first spacer configured to be positioned between the lock and the first leaflet and a second spacer configured to be positioned between the lock and the second leaflet.

Example 19. A method useable for repairing/treating a native heart valve of a subject (e.g., of a living subject or of a simulation), comprising some or all of: (a) inhibiting a first leaflet and a second leaflet from prolapsing with a leaflet coaptation assist element; (b) deploying a first anchor through the first leaflet; (c) deploying a second anchor through the second leaflet; (d) tensioning a first line connected to the first leaflet and a second line connected to the second leaflet to pull the first leaflet and the second leaflet towards each other to a repair position; (e) locking the first line and the second line to hold the first leaflet and the second leaflet in the repair position; and/or (f) removing the leaflet coaptation assist element.

Example 20. The method of example 19, further comprising: (1) delivering the leaflet coaptation assist element, in a delivery state, through a catheter to the native heart valve; (2) positioning the leaflet coaptation assist element between the first leaflet and the second leaflet; and/or (3) moving the leaflet coaptation assist element to a deployed state for inhibiting the first and the second leaflet from prolapsing.

Example 21. The method of any one of examples 19-20, wherein the deploying the first anchor through the first leaflet further comprising deploying the first anchor through an opening in the leaflet coaptation assist element.

Example 22. The method of any one of examples 19-21, further comprising deploying a first spacer to be positioned between the first leaflet and the second leaflet.

Example 23. The method of example 22, wherein the first spacer is configured to receive the first line therethrough.

Example 24. The method of any one of examples 19-23, wherein removing the leaflet coaptation assist element further comprising pulling one or more retrieval lines attached to the leaflet coaptation assist element.

Example 25. The method of example 20, wherein moving the leaflet coaptation assist element to the deployed state further comprises longitudinally compressing a compressible portion of the leaflet coaptation assist element.

Example 26. The method of example 25, wherein the compressible portion defines a spinning top shape when in the deployed state.

Example 27. The method of example 25, wherein longitudinally compressing the compressible portion further comprises holding a distal end of the compressible portion in place while pushing a proximal end of the compressible portion toward the distal end.

Example 28. The method of example 25, wherein longitudinally compressing the compressible portion further comprises includes holding a proximal end of the compressible portion in place while pulling a distal end of the compressible portion toward the proximal end.

Example 29. The method of any one of examples 19-28, further comprising engaging a ventricular side of the first leaflet to position the first leaflet for deploying the first anchor through the first leaflet.

Example 30. A device useable for repairing/treating a native valve, comprising: (a) a coaptation element configured to be positioned between a first leaflet and a second leaflet of the native valve; and/or (b) a first fixation element configured to extend from the coaptation element at a first location and back to the coaptation element at a second location spaced apart from the first location, wherein the first fixation element is configured to capture at least the first leaflet, a first chordae tendineae of the first leaflet, and both between the first fixation element and the coaptation element.

Example 31. The device of example 30, wherein the first fixation element is a wire configured to be moveable in an arc from the first location to the second location.

Example 32. The device of example 31, wherein the first fixation element includes a shape memory alloy.

Example 33. The device of any one of examples 31-32, wherein the first fixation element has a distal piercing tip configured to pierce through the first leaflet.

Example 34. The device of any one of examples 30-33, wherein the first fixation element has a distal end the extends into the coaptation element at the second location.

Example 35. The device of example 34, wherein the distal end is configured to pierce an exterior surface of the coaptation element at the second location.

Example 36. The device of example 34, wherein the coaptation element includes a first pre-formed aperture at the first location and a second pre-formed aperture at the second location.

Example 37. The device of any one of examples 30-36, wherein the first fixation element is configured to capture the first chordae tendineae of the first leaflet without capturing the first leaflet.

Example 38. The device of any one of examples 30-37, further comprising a second fixation element configured to extend from the coaptation element at a third location and back to the coaptation element at a fourth location spaced apart from the third location, wherein the second fixation element is configured to capture at least the second leaflet, a second chordae tendineae of the second leaflet, and both between the second fixation element and the coaptation element.

Example 39. The device of example 38, wherein the first location and the second location are on a first side of the coaptation element and the third location and the fourth location are on a second side of the coaptation element, opposite the first side.

Example 40. A method useable for repairing/treating a native valve of a subject (e.g., of a living subject or of a simulation), comprising: (a) positioning a coaptation element between a first leaflet and a second leaflet of the native valve; (b) moving a first fixation element out of the coaptation element at a first location on the coaptation element; (c) capturing the first leaflet, a first chordae tendineae of the first leaflet, or a combination thereof between the first fixation element and the coaptation element; and (d) moving the first fixation element back to the coaptation element at a second location on the coaptation element.

Example 41. The method of example 40, wherein capturing the first leaflet, the first chordae tendineae of the first leaflet, or a combination thereof further comprises moving the first fixation element in an arc around the first leaflet, the first chordae tendineae of the first leaflet, or a combination thereof.

Example 42. The method of any one of examples 40-41, further comprising piercing the first leaflet with a distal tip of the first fixation element.

Example 43. The method of any one of examples 40-42, further comprising piercing the coaptation element with a distal tip of the first fixation element at the first location.

Example 44. The method of any one of examples 40-43, further comprising piercing the coaptation element with a distal tip of the first fixation element at the second location.

Example 45. The method of any one of examples 40-44, wherein the first chordae tendineae of the first leaflet is captured without capturing the first leaflet.

Example 46. The method of any one of examples 40-45, further comprising: (1) moving a second fixation element out of the coaptation element at a third location on the coaptation element; (2) capturing the second leaflet, a second chordae tendineae of the second leaflet, or a combination thereof between the second fixation element and the coaptation element; and (3) moving the second fixation element back to the coaptation element at a fourth location on the coaptation element.

Example 47. The method of example 46, wherein the first location and the second location are on a first side of the coaptation element, and the third location and the fourth location are on a second side of the coaptation element, opposite the first side.

Example 48. A device useable for repairing/treating anatomy, comprising: (a) a first capture element configured to engage a first portion of the anatomy, which can optionally be a first leaflet, multiple first chordae tendineae of a first leaflet, or other tissue; (b) a second capture element configured to engage a second portion of the anatomy, which can optionally be a second leaflet, multiple second chordae tendineae of a second leaflet, or other tissue; and/or (c) wherein the first capture element and the second capture element are configured to be drawn toward each other to pull the first portion (e.g., multiple first chordae tendineae of the first leaflet, etc.) and the second portion (e.g., multiple second chordae tendineae of the second leaflet, etc.) toward each other.

Example 49. The device of example 48, wherein the first capture element is configured as a first cylindrical rod and the second capture element is configured as a second rod.

Example 50. The device of any one of examples 48-49, wherein the first capture element has a first end and a second end opposite the first end and the second capture element has a third end and a fourth end opposite the third end.

Example 51. The device of example 50, further comprising a first attachment element connecting the first end to the third end, wherein the first attachment element is configured to draw the first end toward the third end.

Example 52. The device of example 51, wherein the first attachment element includes a suture line.

Example 53. The device of any one of examples 50-52, further comprising a second attachment element connecting the second end to the fourth end, wherein the second attachment element is configured to draw the second end toward the fourth end.

Example 54. The device of any one of examples 50-53, wherein the second end is connected to the fourth end by a joint portion.

Example 55. The device of example 54, wherein the joint portion includes a hinge.

Example 56. The device of any one of examples 54-55, wherein the first capture element and the second capture element are configured to pivot between an open position and a repair position.

Example 57. The device of any one of examples 54-56, wherein the first end and the third end are configured to attach.

Example 58. The device of example 57, wherein the first end includes a latch configured to attach to a projection on the third end.

Example 59. A method useable for repairing/treating anatomy of a subject (e.g., of a living subject or of a simulation), comprising: (a) positioning a first capture element to engage a first tissue, which can optionally be first chordae tendineae of a first leaflet of a native heart valve; (b) positioning a second capture element to engage a second tissue, which can optionally be second chordae tendineae of a second leaflet of the native heart valve; and/or (c) moving the first capture element toward the second capture element to pull the first tissue (e.g., first chordae tendineae, etc.) toward the second tissue (e.g., second chordae tendineae, etc.) to a repair position.

Example 60. The method of example 59, wherein the moving the first capture element toward the second capture element further comprises pivoting the first capture element relative to the second capture element.

Example 61. The method of example 60, further comprising latching the first capture element to the second capture element in the repair position.

Example 62. The method of any one of examples 60-61, further comprising securing the first capture element to the second capture element in the repair position with one or more attachment elements.

Example 63. The method of example 62, wherein the one or more attachment elements include a suture line.

Example 64. The method of any one of examples 59-63, further comprising connecting the first capture element to the second capture element by one or more attachment elements.

Example 65. The method of example 64, where the one or more attachment elements include a suture line, and wherein moving the first capture element toward the second capture element includes cinching the suture line.

Example 66. The method of any one of examples 64-65, wherein connecting the first capture element to the second capture element by the one or more attachment elements further comprising connecting a first end of the first capture element to a third end of the second capture element with one of the one or more attachment elements and connecting a second end of the first capture element to a fourth end of the second capture element with another of the one or more attachment elements.

Example 67. A device useable for repairing/treating a native heart valve, comprising: (a) a frame including a plurality of struts; and/or (b) means for attaching to leaflet tissue positioned on (e.g., in contact with) the plurality of struts.

Example 68. The device of example 67, wherein the means for attaching to leaflet tissue comprises a plurality of spikes.

Example 69. The device of example 68, wherein the plurality of struts define a plane and wherein the plurality of spikes extend coplanar to the plane.

Example 70. The device of example 69, wherein the plurality of spikes extend from both a first side and a second side of at least one of the plurality of struts.

Example 71. The device of any one of examples 69-70, wherein the plurality of spikes extend from only one side of at least one of the plurality of struts.

Example 72. The device of any one of examples 67-71, wherein the plurality of struts define one or more diamond shapes.

Example 73. The device of any one of examples 67-72, wherein the plurality of struts define one or more rectangular shapes.

Example 74. The device of any one of examples 67-73, wherein the plurality of struts define one or more circular shapes.

Example 75. The device of any one of examples 67-74, wherein the frame is configured to be folded along a mid-line prior to being positioned between a first leaflet and a second leaflet of the native heart valve.

Example 76. A method of repairing/treating a native heart valve of a subject (e.g., of a living subject or of a simulation); comprising: (a) positioning a repair device between a first leaflet and a second leaflet of the native heart valve such that the first leaflet and the second leaflet contact the repair device during systole; and/or (b) passively attaching the first leaflet and the second leaflet to the repair device upon the first leaflet and the second leaflet contacting the repair device.

Example 77. The method of example 76, wherein passively attaching the first leaflet to the repair device further comprises a portion of the repair device penetrating into the first leaflet.

Example 78. The method of example 77, wherein the portion of the repair device comprises one or more spikes.

Example 79. A device useable for repairing/treating anatomy, comprising: (a) a pair of paddles comprising a first paddle and a second paddle, wherein the pair of paddles are movable between an open position and a closed position; (b) a first capture element attached to the first paddle, the first capture element being movable between an elongated configuration and an expanded configuration, wherein the first capture element is configured to engage first tissue (e.g., multiple first chordae tendineae of a first leaflet of a heart valve, etc.) when in the elongated configuration; (c) a second capture element attached to the second paddle, the second capture element being movable between an elongated configuration and an expanded configuration, wherein the second capture element is configured to engage second tissue (e.g., multiple second chordae tendineae of a second leaflet of the heart valve, etc.); and/or (d) wherein movement of the pair of paddles to the closed position causes the first capture element and the second capture element to be drawn toward each other to pull the first tissue (e.g., multiple first chordae tendineae of the first leaflet, etc.) and the second tissue (e.g., multiple second chordae tendineae of the second leaflet, etc.) toward each other.

Example 80. The device according to example 79, wherein the first capture element comprises a first connection element, a second connection element, a first flexible portion, and a second flexible portion, wherein the first and second flexible portions are each connected to both of the first and second connection elements.

Example 81. The device according to example 80, wherein at least one of the first connection element and the second connection element are movable within a slot of the first paddle to move the first capture element between the elongated and expanded configurations.

Example 82. The device according to any of examples 80-81, wherein the first connection element is a first distance away from the second connection element when the first capture element is in the elongated configuration and the first connection element is a second distance away from the second connection element when the first capture element is in the expanded configuration, and wherein the first distance is greater than the second distance.

Example 83. The device according to any of examples 80-82, wherein the first connection element, the second connection element, the first flexible portion, and the second flexible portion are integrally formed.

Example 84. The device according to any of examples 79-83, wherein the first and second capture elements comprise Nitinol.

Example 85. The device according to any of examples 79-84, wherein the first and second capture elements are normally in the expanded configuration.

Example 86. The device according to any of examples 79-85, wherein the first and second paddle are integrally formed.

Example 87. The device according to any of examples 79-86, wherein the first paddle comprises a first hinge portion and the second paddle comprises a second hinge portion, and wherein the first and second hinge portions allow for the first and second paddles to be moved between the open and closed positions.

Example 88. The device according any of examples 79-87, wherein the first and second paddles comprise Nitinol.

Example 89. The device according to any of examples 84-88, wherein the first paddle, the second paddle, the first capture element, and the second capture element are integrally formed.

Example 90. A method useable for repairing/treating a native valve of a subject (e.g., of a living subject or of a simulation), the method comprising: (a) positioning a valve repair device proximate the native valve, the valve repair device comprising a first paddle, a second paddle, a first capture element connected to the first paddle, and a second capture element connected to the second paddle; (b) positioning the first capture element to engage a first tissue (e.g., chordae tendineae of a first leaflet, etc.) of the native valve, wherein the first capture element is in an elongated configuration; (c) positioning the second capture element to engage a second tissue (e.g., second chordae tendineae of a second leaflet, etc.) of the native valve, wherein the second capture element is in an elongated configuration; (d) moving the first capture element to an expanded configuration such that the first capture element extends behind multiple chordae of the native valve (e.g., of first chordae tendineae); (e) moving the second capture element to an expanded configuration such that the second capture element extends behind multiple chordae of the native valve (e.g., of second chordae tendineae); and/or (f) moving the first paddle and the second paddle to a closed position such that the first and second capture elements move toward each other and pull the first chordae tendineae toward the second chordae tendineae.

Example 91. The method according to example 90, wherein the first capture element comprises a first connection element, a second connection element, a first flexible portion, and a second flexible portion, wherein the first and second flexible portions are each connected to both of the first and second connection elements.

Example 92. The method according to example 91, further comprising moving at least one of the first connection element and the second connection element within a slot of the first paddle to move the first capture element from the elongated configuration to the expanded configuration.

Example 93. The method according to any of examples 91-92, wherein the first connection element is a first distance away from the second connection element when the first capture element is in the elongated configuration and the first connection element is a second distance away from the second connection element when the first capture element is in the expanded configuration, and wherein the first distance is greater than the second distance.

Example 94. The method according to any of examples 91-93, wherein the first connection element, the second connection element, the first flexible portion, and the second flexible portion are integrally formed.

Example 95. The method according to any of examples 90-94, wherein the first and second capture elements comprise Nitinol.

Example 96. The method according to any of examples 90-95, wherein the first and second capture elements are normally in the expanded configuration.

Example 97. The method according to example 96, further comprising engaging the first capture element with one or more actuation elements to move the first capture element to the elongated configuration prior to positioning the first capture element to engage the first chordae tendineae.

Example 98. The method according to example 90, wherein moving the first paddle and the second paddle to the closed position comprises engaging one or more hinge portions of the first and second paddle with an actuation element.

Example 99. The method according to any of examples 90-98, wherein the first and second paddle are integrally formed.

Example 100. The method according any of examples 90-99, wherein the first and second paddles comprise Nitinol.

Example 101. The method according to any of examples 95-100, wherein the first paddle, the second paddle, the first capture element, and the second capture element are integrally formed.

Example 102. The method according to example 101, further comprising pulling a free end of the first capture element to move the first capture element to the elongated configuration, and further comprising pulling a free end of the second capture element to move the second capture element to the elongated configuration.

Example 103. A device useable for repairing/treating a native valve, comprising: (a) a first capture element, the first capture element comprising a first engagement portion and a second engagement portion, wherein the first engagement portion is configured to engage a first portion of a first tissue (e.g., first chordae tendineae of a first leaflet, etc.) of the native valve, wherein the second engagement portion is configured to engage a first portion of a second tissue (e.g., second chordae tendineae of a second leaflet, etc.) of the native valve; (b) a second capture element, the second capture element comprising a first engagement portion and a second engagement portion, wherein the first engagement portion is configured to engage a second portion of the first tissue (e.g., of first chordae tendineae of the first leaflet, etc.) of the heart valve, wherein the second engagement portion is configured to engage a second portion of the second tissue (e.g., of second chordae tendineae of a second leaflet, etc.) of the native heart valve; (c) wherein the first and second capture elements are rotatably coupled such that the first and second capture elements are movable between a delivery configuration and a capture configuration; (d) wherein the first engagement portion of the first capture element and the second engagement portion of the second capture element secure the first portion of the first tissue (e.g., first chordae tendineae, etc.) and the second portion of the second tissue (e.g., second chordae tendineae, etc.) in a first capture area when in the capture configuration; and/or (e) wherein the second engagement portion of the first capture element and the first engagement portion of the second capture element secure the first portion of the second tissue (e.g., second chordae tendineae, etc.) and the second portion of the first tissue (e.g., first chordae tendineae, etc.) in a second capture area when in the capture configuration.

Example 104. The device according to example 103, wherein the first and second capture elements are rotatably connected by a hinge connection.

Example 105. The device according to any of examples 103-104, further comprising a hub portion connected to each of the first and second capture elements.

Example 106. The device according to example 105, wherein the hub portion is configured to receive an actuation element for moving the first and second capture elements between delivery and capture configurations.

Example 107. The device according to any of examples 103-106, wherein the first capture element is configured to rotate in a counterclockwise direction to move from the delivery configuration to the capture configuration, and wherein the second capture element is configured to rotate in a clockwise direction to move from the delivery configuration to the capture configuration.

Example 108. The device according to any of examples 103-107, wherein the first and second engagement portions of the first capture element are integrally formed.

Example 109. The device according to any of examples 103-108, wherein the first and second capture elements comprise Nitinol.

Example 110. The device according to any of examples 103-109, wherein each of the first and second capture elements are configured to rotate between about 75 degrees and about 105 degrees to move from the delivery configuration to the capture configuration.

Example 111. The device according to any of examples 103-110, wherein the first and second capture elements are normally in the capture configuration.

Example 112. The device according to any of examples 103-111, wherein the first engagement portion of the first capture element is longer than the second engagement portion of the first capture element, and wherein the second engagement portion of the second capture element is longer than the first engagement portion of the second capture element.

Example 113. A method useable for repairing/treating a native valve of a subject (e.g., of a living subject or of a simulation), the method comprising: (a) positioning a repair/treatment device in a delivery configuration proximate a gap between adjacent leaflets of the native heart valve, the repair/treatment device comprising a first capture element having a first engagement portion and a second engagement and a second capture element having a first engagement portion and a second engagement portion, wherein the first and second engagement portions of each of the first and second capture elements are positioned substantially perpendicular to the gap between the adjacent leaflets when in the delivery configuration; (b) rotating the first and second capture elements to a capture configuration; (c) wherein the rotating the first capture element comprising rotating the first capture element in a first direction such that the first engagement portion engages a first portion of a first tissue (e.g., of first chordae tendineae of a first leaflet, etc.) of the native valve and the second engagement portion engages a first portion of a second tissue (e.g., of second chordae tendineae of a second leaflet, etc.) of the native valve; (d) wherein the rotating the second capture element comprises rotating the second capture element in a second direction that is opposite the first direction such that the first engagement portion engages a second portion of the first tissue (e.g., of first chordae tendineae of the first leaflet, etc.) of the native valve and the second engagement portion engages a second portion of the second tissue (e.g., of second chordae tendineae of the second leaflet, etc.) of the native valve; (e) wherein the first engagement portion of the first capture element and the second engagement portion of the second capture element secure the first portion of the first tissue (e.g., of first chordae tendineae, etc.) and the second portion of the second tissue (e.g., of second chordae tendineae, etc.) in a first capture area when in the capture configuration; and/or (f) wherein the second engagement portion of the first capture element and the first engagement portion of the second capture element secure the first portion of the second tissue (e.g., of second chordae tendineae, etc.) and the second portion of the first tissue (e.g., of first chordae tendineae, etc.) in a second capture area when in the capture configuration.

Example 114. The method according to example 113, wherein the first and second capture elements are rotatably connected by a hinge connection.

Example 115. The method according to any of examples 113-114, wherein the repair/treatment device further comprises a hub portion connected to each of the first and second capture elements.

Example 116. The method according to example 115, wherein the hub portion is configured to receive an actuation element for moving the first and second capture elements between delivery and capture configurations.

Example 117. The method according to any of examples 113-116, wherein the first direction is a counterclockwise direction, and the second direction is a clockwise direction.

Example 118. The method according to any of examples 113-117, wherein the first and second engagement portions of the first capture element are integrally formed.

Example 119. The method according to any of examples 113-118, wherein the first and second capture elements comprise Nitinol.

Example 120. The method according to any of examples 113-119, wherein each of the first and second capture elements are configured to rotate between about 75 degrees and about 105 degrees to move from the delivery configuration to the capture configuration.

Example 121. The method according to any of examples 113-120, wherein the first and second capture elements are normally in the capture configuration.

Example 122. The method according to any of examples 113-121, wherein the first engagement portion of the first capture element is longer than the second engagement portion of the first capture element, and wherein the second engagement portion of the second capture element is longer than the first engagement portion of the second capture element.

Example 123. A device useable for repairing or treating anatomy, comprising: (a) a base portion; (b) a plurality of capture elements, each of the capture elements are pivotably connected to the base portion such that each of the capture elements are movable between a delivery configuration and a capture configuration, the plurality of capture elements comprising a first capture element, a second capture element, a third capture element, and a fourth capture element; (c) wherein the first capture element is configured to engage a first portion of a first tissue (e.g., of first chordae tendineae of a first leaflet of a native valve, etc.) when moved from the delivery configuration to the capture configuration; (d) wherein the second capture element is configured to engage a first portion of a second tissue (e.g., of second chordae tendineae of a second leaflet of the native valve, etc.) when moved from the delivery configuration to the capture configuration; (e) wherein the third capture element is configured to engage a second portion of the first tissue (e.g., of first chordae tendineae of the first leaflet, etc.) when moved from the delivery configuration to the capture configuration; (f) wherein the fourth capture element is configured to engage a second portion of the second tissue (e.g., of second chordae tendineae of the second leaflet, etc.) when moved from the delivery configuration to the capture configuration; (g) wherein the first and second capture elements secure the first portion of the first tissue (e.g., of first chordae tendineae, etc.) and the first portion of the second tissue (e.g., of second chordae tendineae, etc.) in a first capture area when in the capture configuration; and/or (h) wherein the third and fourth capture elements secure the second portion of the first tissue (e.g., of first chordae tendineae, etc.) and the second portion of the second tissue (e.g., of second chordae tendineae, etc.) in a second capture area when in the capture configuration.

Example 124. The device according to example 123, wherein the first and third capture elements are integrally formed, and wherein the second and fourth capture elements are integrally formed.

Example 125. The device according to any of examples 123-124, wherein the base portion is configured to receive one or more actuation elements for moving the plurality of capture elements between delivery and capture configurations.

Example 126. The device according to any of examples 123-125, wherein each of the capture elements comprise Nitinol.

Example 127. The device according to any of examples 123-126, wherein each of the capture elements are normally in the capture configuration.

Example 128. The device according to any of examples 123-127, wherein each of the capture elements can be moved from the delivery configuration to the capture configuration independently.

Example 129. A method useable for repairing/treating a native valve of a subject (e.g., of a living subject or of a simulation), the method comprising: (a) positioning a repair/treatment device in a delivery configuration proximate a gap or space between first and second portions of the native valve (e.g., proximate a gap or space between adjacent leaflets of the native valve, etc.), the repair/treatment device comprising a base portion a plurality of capture elements pivotably attached to the base portion, wherein each of the capture elements are positioned substantially perpendicular to the gap/space between the first and second portions of the native valve (e.g., between adjacent leaflets, etc.) when in the delivery configuration; (b) pivoting a pair of the capture elements toward each other to a capture configuration such that the pair of capture elements define a capture area; (c) wherein a first capture element of the pair of capture elements engages a portion of a first tissue (e.g., of first chordae tendineae of a first leaflet, etc.) of the native valve and a second capture element of the pair of capture elements engages a portion of a second tissue (e.g., of second chordae tendineae of a second leaflet, etc.) of the native valve; and/or (d) wherein the portion of the first tissue (e.g., chordae tendineae, etc.) and the portion of the second tissue (e.g., second chordae tendineae, etc.) are secured within the capture area.

Example 130. The method according to example 129, further comprising pivoting a second pair of the capture elements toward each other to the capture configuration such that the second pair of capture elements define a second capture area, wherein a third capture element of the second pair of capture elements engages a second portion of the first tissue and a fourth capture element of the second pair of capture elements engages a second portion of the second tissue, and wherein the second portion of the first tissue and the second portion of the second tissue are secured within the second capture area.

Example 131. The method according to example 130, wherein the first and third capture elements are integrally formed, and wherein the second and fourth capture elements are integrally formed.

Example 132. The method according to any of examples 129-131, wherein the base portion is configured to receive one or more actuation elements for moving the plurality of capture elements between delivery and capture configurations.

Example 133. The method according to any of examples 129-132, wherein each of the capture elements comprise Nitinol.

Example 134. The method according to any of examples 129-133, wherein each of the capture elements are normally in the capture configuration.

Example 135. The method according to any of examples 129-134, wherein each of the capture elements can be moved from the delivery configuration to the capture configuration independently.

Example 136. A device useable for repairing or treating anatomy, comprising: (a) a pair of paddles comprising a first paddle and a second paddle, wherein the pair of paddles are movable between an open position and a closed position; (b) a first capture element attached to the first paddle, the first capture element comprising a first housing and a first engagement element at least partially disposed within the first housing, wherein the first engagement element is movable between a compressed configuration and an expanded configuration; (c) a second capture element attached to the second paddle, the second capture element comprising a second housing and a second engagement element at least partially disposed within the second housing, wherein the second engagement element is movable between a compressed configuration and an expanded configuration; and/or (d) wherein movement of the pair of paddles to the closed position causes the first engagement element of the first capture element to engage a first tissue (e.g., to engage multiple first chordae tendineae of a first leaflet of a native valve, etc.) when in the expanded configuration and causes the second engagement element of the second capture element to engage a second tissue (e.g., to engage multiple second chordae tendineae of a second leaflet of a native valve, etc.) when in the expanded configuration.

Example 137. The device according to example 136, wherein the first engagement portion of the first capture element comprises a first engagement portion and a second engagement portion, wherein the first and second engagement portions are compressed toward each other when the first engagement element is in the compressed configuration, and wherein the first and second engagement portions extend away from each other when the first engagement element is in the expanded configuration.

Example 138. The device according to any of examples 136-137, wherein the first housing comprises a channel for receiving and engaging the first engagement element.

Example 139. The device according to example 138, wherein the channel comprises one or more sloped walls for facilitating movement of the first engagement element between the compressed and expanded configurations.

Example 140. The device according to any one of examples 137-139, wherein the first housing has a first opening for allowing the first engagement portion of the first engagement element to move to the expanded configuration and a second opening for allowing the second engagement portion of the first engagement element to move to the expanded configuration.

Example 141. The device according to any of examples 136-140, wherein each of the first paddle and the second paddle comprises an inner paddle portion and an outer paddle portion.

Example 142. The device according to any of examples 136-141, further comprising a first paddle frame connected to the first paddle and a cap, and further comprising second paddle frame connected to the second paddle and the cap.

Example 143. A method useable for repairing/treating a native valve of a subject (e.g., of a living subject or of a simulation), the method comprising: (a) positioning a repair/treatment device proximate the native valve, the repair/treatment device comprising a first paddle, a second paddle, a first capture element connected to the first paddle, and a second capture element connected to the second paddle, wherein the first capture element comprise a first housing and a first engagement element that is movable between a compressed position and an expanded position relative to the first housing, and wherein the second capture element comprise a second housing and a second engagement element that is movable between a compressed position and an expanded position relative to the second housing; (b) positioning the first capture element to engage a first tissue (e.g., to engage first chordae tendineae of a first leaflet, etc.) of the native valve, wherein the first capture element is in the compressed; positioning the second capture element to engage a second tissue (e.g., to engage second chordae tendineae of a second leaflet, etc.) of the native valve, wherein the second capture element is in the compressed configuration; (c) moving the first capture element to the expanded configuration such that the first engagement element of the first capture element extends behind multiple chordae associated with the first tissue; (d) moving the second capture element to the expanded configuration such that the second engagement element of the second capture element extends behind multiple chordae associated with the second tissue; and (e) moving the first paddle and the second paddle to a closed position such that the first and second capture elements move toward each other and pull the first tissue and the second tissue closer together.

Example 144. The method according to example 143, wherein the first engagement portion of the first capture element comprises a first engagement portion and a second engagement portion, wherein the first and second engagement portions are compressed toward each other when the first engagement element is in the compressed configuration, and wherein the first and second engagement portions extend away from each other when the first engagement element is in the expanded configuration.

Example 145. The method according to any of examples 143-144, wherein the first housing comprises a channel for receiving and engaging the first engagement element.

Example 146. The method according to example 145, wherein the channel comprises one or more sloped walls for facilitating movement of the first engagement element between the compressed and expanded configurations.

Example 147. The method according to any one of examples 144-146, wherein the first housing has a first opening for allowing the first engagement portion of the first engagement element to move to the expanded configuration and a second opening for allowing the second engagement portion of the first engagement element to move to the expanded configuration.

Example 148. The method according to any of examples 143-147, wherein each of the first paddle and the second paddle comprises an inner paddle portion and an outer paddle portion.

Example 149. The device according to any of examples 143-148, further comprising a first paddle frame connected to the first paddle and a cap, and further comprising second paddle frame connected to the second paddle and the cap.

Example 150. A device useable for repairing/treating a native valve or other anatomy, comprising: (a) a first clip member capable of being attached to a first portion of a leaflet of the native valve (or to a first portion of tissue of other anatomy), the first clip member having a first clip body and a first attachment portion; (b) a second clip member capable of being attached to a second portion of the leaflet of the native valve (or to a second portion of tissue of other anatomy, or to a second portion of a different leaflet of the native valve, etc.) second clip member a second clip body and a second attachment portion; (c) wherein the first clip member and the second clip member are movable between a disengaged position and an engaged position relative to each other; and/or (d) wherein the first and second clip members are configured such that movement of the first and second clip members to the engaged position after the first clip member is attached to the first portion of the leaflet and the second clip member is attached to the second portion of the leaflet causes a region (e.g., a gap, cleft, fold, flap, stretch, space, etc.) between the first and second portions to be moved closer together or closed.

Example 151. The device according to example 150, further comprising one or more actuation elements for moving the first and second clip members from the disengaged position to the engaged position.

Example 152. The device according to example 151, wherein the one or more actuation elements comprises one or more springs that cause the first and second clip members to be in a normally engaged position.

Example 153. The device according to example 151, wherein the one or more actuation elements comprise a shape set material that causes the first and second clip members to be in a normally closed position.

Example 154. The device according to example 151, wherein the one or more actuation elements comprises NiTi wire that is configured to move the first and second clip members to the engaged position when heated.

Example 155. The device according to example 151, wherein the one or more actuation elements comprise a bifurcated member and a tube that engages the bifurcated member to move the first and second clip members to the engaged position.

Example 156. The device according to example 151, wherein the one or more actuation elements comprise a suture that is threaded through each of the first and second clip members.

Example 157. The device according to any of examples 150-156, at least one of the first and second clip members has one or more locking features that secure the first clip member to the second clip member when the first and second clip members are in the engaged position.

Example 158. The device according to example 157, wherein the first clip member has a first locking protrusion and a first locking receptacle, wherein the second clip member has a second locking protrusion and a second locking receptacle, wherein the first locking receptacle is configured to receive the second locking protrusion, and wherein the second locking receptacle is configured to receive the first locking protrusion.

Example 159. Any combination or subcombination of any of the features of any of the previous examples.

Any of the various systems, assemblies, devices, components, apparatuses, etc. in this disclosure (including in the examples above) can be sterilized (e.g., with heat, radiation, ethylene oxide, hydrogen peroxide, etc.) to ensure they are safe for use with patients, and the methods herein can comprise (or additional methods comprise or consist of) sterilization of the associated system, device, component, apparatus, etc. (e.g., with heat, radiation, ethylene oxide, hydrogen peroxide, etc.).

While various inventive aspects, concepts and features of the disclosures can be described and illustrated herein as embodied in combination in the examples herein, these various aspects, concepts, and features can be used in many alternative examples, either individually or in various combinations and sub-combinations thereof. Unless expressly excluded herein all such combinations and sub-combinations are intended to be within the scope of the present application. Still further, while various alternative examples as to the various aspects, concepts, and features of the disclosures—such as alternative materials, structures, configurations, methods, devices, and components, alternatives as to form, fit, and function, and so on—may be described herein, such descriptions are not intended to be a complete or exhaustive list of available alternative examples, whether presently known or later developed. Those skilled in the art can readily adopt one or more of the inventive aspects, concepts, or features into additional examples and uses within the scope of the present application even if such examples are not expressly disclosed herein.

Additionally, even though some features, concepts, or aspects of the disclosures may be described herein as being a preferred arrangement or method, such description is not intended to suggest that such feature is required or necessary unless expressly so stated. Still further, example or representative values and ranges may be included to assist in understanding the present application, however, such values and ranges are not to be construed in a limiting sense and are intended to be critical values or ranges only if so expressly stated.

Moreover, while various aspects, features and concepts may be expressly identified herein as being inventive or forming part of a disclosure, such identification is not intended to be exclusive, but rather there may be inventive aspects, concepts, and features that are fully described herein without being expressly identified as such or as part of a specific disclosure, the disclosures instead being set forth in the appended claims. Descriptions of example methods or processes are not limited to inclusion of all steps as being required in all cases, nor is the order that the steps are presented to be construed as required or necessary unless expressly so stated. The words used in the claims have their full ordinary meanings and are not limited in any way by the description of the examples in the specification.

What is claimed is:

1. A device useable for repairing or treating a heart valve, comprising:
a first capture element configured to engage a first chordea tendinea;
a second capture element configured to engage a second chordea tendinea; and
wherein the first capture element and the second capture element are configured to be drawn toward each other to pull the first chordea tendinea and the second chordea tendinea toward each other.

2. The device of claim 1, wherein the first capture element is configured as a first rod and the second capture element is configured as a second rod.

3. The device of claim 1, wherein the first capture element has a first end and a second end opposite the first end and the second capture element has a third end and a fourth end opposite the third end.

4. The device of claim 3, further comprising a first attachment element connecting the first end to the third end, wherein the first attachment element is configured to draw the first end toward the third end.

5. The device of claim 3, further comprising a second attachment element connecting the second end to the fourth end, wherein the second attachment element is configured to draw the second end toward the fourth end.

6. The device of claim 5, wherein the second end is connected to the fourth end by a joint portion.

7. The device of claim 2, wherein the first end includes a latch configured to attach to a projection on the third end.

8. A device useable for repairing or treating a native heart valve, comprising:
a first clip member for attaching to a first portion of a leaflet of the native heart valve, the first clip member having a first clip body and a first attachment portion;
a second clip member for attaching to a second portion of the leaflet of the native heart valve, the second clip member having a second clip body and a second attachment portion;
wherein the first clip member and the second clip member are movable between a disengaged position and an engaged position relative to each other; and
wherein the first and second clip members are configured such that movement of the first and second clip members to the engaged position after the first clip member is attached to the first portion of the leaflet and the second clip member is attached to the second portion of the leaflet causes a gap or cleft between the first and second portions of the leaflet to be closed to prevent regurgitation therethrough.

9. The device according to claim 8, further comprising one or more actuation elements for moving the first and second clip members from the disengaged position to the engaged position.

10. The device according to claim 8, wherein the one or more actuation elements comprises one or more springs that cause the first and second clip members to be in a normally engaged position.

11. The device according to claim 8, wherein the one or more actuation elements comprise a bifurcated member and a tube that engages the bifurcated member to move the first and second clip members to the engaged position.

12. A device useable for repairing a native valve, comprising:
a leaflet coaptation assist element configured to be positioned between a first leaflet and a second leaflet of the native valve to inhibit the first leaflet and the second leaflet from prolapsing;
a first anchor configured to engage a first ventricular side of the first leaflet;
a first line connected to the first anchor and extending through the first leaflet to a first atrial side of the first leaflet;
a second anchor configured to engage a second ventricular side of the second leaflet;
a second line connected to the second anchor and extending through the second leaflet to a second atrial side of the second leaflet, wherein the first line, the first anchor, the second line, and the second anchor are configured to pull the first leaflet and the second leaflet into a repair position;
a lock configured to engage the first line and the second line to secure the first leaflet and the second leaflet in the repair position;
wherein the leaflet coaptation assist element is configured to be removed after the lock has locked the first leaflet and the second leaflet; and
wherein the leaflet coaptation assist element has a delivery state for deploying through a catheter and a deployed state for positioning in the native valve between the first leaflet and the second leaflet.

13. The device of claim 12, wherein the leaflet coaptation assist element includes a first arm connected to a second arm by a joint portion.

14. The device of claim 13, wherein the first arm has a first outer surface for engaging the first leaflet and the second arm has a second outer surface of engaging the second leaflet.

15. The device of claim 13, wherein the leaflet coaptation assist element is configured for the first line to extend through the first arm and the second line to extend through the second arm.

16. The device of claim 13, further comprising a first spacer configured to be positioned between the lock and the first leaflet and a second spacer configured to be positioned between the lock and the second leaflet.

17. The device of claim 13, further comprising a first retrieval line attached to the first arm and a second retrieval line attached to the second arm to withdraw the leaflet coaptation assist element from between the first leaflet and the second leaflet after the lock is in place.

18. A device useable for repairing a native heart valve, comprising:
a frame including a plurality of struts; and
a plurality of spikes for attaching to leaflet tissue positioned on the plurality of struts.

19. The device of claim 18, wherein the plurality of struts define a plane and wherein the plurality of spikes extend coplanar to the plane.

20. The device of claim 18, wherein the frame is configured to be folded along a mid-line prior to being positioned between a first leaflet and a second leaflet of the native heart valve.
